SEQUENCE LISTING (42 sequences)

SEQ ID NO: 1 - DNA, 21 bp, Artificial Sequence
  Description of Artificial Sequence Synthetic primer
gtccctgccctttgtacacac

SEQ ID NO: 2 - DNA, 23 bp, Artificial Sequence
  Description of Artificial Sequence Synthetic primer
ttgatatgcttaagttcagcggg

SEQ ID NO: 3 - DNA, 710 bp, Rhodotorula glutinis
cgcccgtcgctactaccgattgaatggcttagtgaggcctccggattggctattgggagctcgcgagagcacctgactgccgagaagttgtacgaacttggtcatttagaggaagtaaaagtcgtaacaaggtttccgtaggtgaacctgcggaaggatcattagtgaatattagggtgtccaacttaacttggagcccgaccctcactttctaaccctgtgcatttgtcttgggtagtagcttgcgtcagcgagcgaatcccatttcacttacaaacacaaagtctatgaatgtaacaaatttataacaaaacaaaactttcaacaacggatctcttggctctcgcatcgatgaagaacgcagcgaaatgcgatacgtaatgtgaattgcagaattcagtgaatcatcgaatctttgaacgcaccttgcgctccatggtattccgtggagcatgcctgtttgagtgtcatgaattcttcaacccacctctttcttagtgaatcaggcggtgtttggattctgagcgctgctggcttcgcggcctagctcgctcgtaatgcattagcatccgcaatcgaacttcggattgactcggcgtaatagactattcgttgaggattctggtctctgactggagccgggtaaggttaaagggagctactaatcctcatgtctatcttgagattagacctcaaatcaggtaggacta

SEQ ID NO: 4 - DNA, 710 bp, Lipomyces tetrasporus; identical to SEQ ID NO: 3

SEQ ID NO: 5 - DNA, 707 bp, Rhodotorula glutinis
cgcccgtcgctactaccgattgaatggcttagtgaggcctccggattggctattgggagctcgcgagagcacccgactgccgagaagttgtacgaacttggtcatttagaggaagtaaaagtcgtaacaaggtttccgtaggtgaacctgcggaaggatcattagtgaatattagggcgtccaacttaacttggagcccgaactctcactttctaaccctgtgcatctgtttctggtcagtagctctctcgggagtgaacgccattcacttaaaacacaaagtctatgaatgtataaaatttataacaaaacaaaactttcaacaacggatctcttggctctcgcatcgatgaagaacgcagcgaaatgcgataagtaatgtgaattgcagaattcagtgaatcatcgaatctttgaacgcaccttgcgctctctggtattccggagagcatgcctgtttgagtgtcatgaaatcttcaaccctctcttttcttaatgaatcgagaggtgcttggatcctgagcgctgctggcttcggcctagctcgttcgtaatgcattagcatccgcaatcgaacttcggattgacttggcgtaatagactattcgctgaggattctggtctcgtaccagagccgggttgggttaaaggaagcttctaatcctaaaagtctaacttttgattagatctcaaatcaggtaggacta

SEQ ID NO: 6 - DNA, 707 bp, Lipomyces tetrasporus; identical to SEQ ID NO: 5

SEQ ID NO: 7 - DNA, 630 bp, Lipomyces tetrasporus
cgcccgtcgctactaccgattgaatggcttagtgaggccttcggactggctccagaaaatgggaaaccattatcaggagctggaaagttggtcaaacttggtcatttagaggaagtaaaagtcgtaacaaggtttccgtaggtgaacctgcggaaggatcattactgagtatttgtcttttaaagacatctctctatccataaactcttttttctaaaaagacatgatttacacaattagtctgaatgattatataaaaatcttcaaaactttcaacaacggatctcttggttctcgcatcgatgaagaacgcagcaaaatgcgataagtattgtgaattgcaggattttgtgaatcatcgaatttttgaacgcacattgcaccttctggtattccggagggtatacctgtttgagcgtcatttatatactcaaaactttgttttggtgatgggcacatatctggtgagagctagatttgcctgaaatatagtggtagagattgctacgagttatgcaagttagccaatgctattaagttaattcgttggtgaagcatgcggagctttagcggtcgccttccttaactattggaatttttctaattttgacctcaaatcaggcaggagta

SEQ ID NO: 8 - DNA, 641 bp, Lipomyces starkeyi
cgcccgtcgctactaccgattgaatggcttagtgagaccctcggattggcgttaggaagccggcaacggcatcctttggccgagaagttggtcaaacttggtcatttagaggaagtaaaagtcgtaacaaggtttccgtaggtgaacctgcggaaggatcattagtgattgcctttataggcttataactatatccacttacacctgtgaactgttctattacttgacgcaagtcgagtatttttacaaacaatgtgtaatgaacgtcgttttattataacaaaataaaactttcaacaacggatctcttggctctcgcatcgatgaagaacgcagcgaattgcgataagtaatgtgaattgcagaattcagtgaatcatcgaatctttgaacgcagcttgcgctctctggtattccggagagcatgcctgtttcagtgtcatgaaatctcaaccactagggtttcctaatggattggatttgggcgtctgcgatctctgatcgctcgccttaaaagagttagcaagtttgacattaatgtctggtgtaataagtttcactgggtccattgtgttgaagcgtgcttctaatcgtccgcaaggacaattactttgactctggcctgaaatcaggtaggacta

SEQ ID NO: 9 - DNA, 641 bp, Trichosporon montevideense; identical to SEQ ID NO: 8

SEQ ID NO: 10 - DNA, 456 bp, Yarrowia lipolytica
cgcccgtcgctactaccgattgaatggtttagtgagaccttgggagggcgagatgaggggggcaaccccttttgaacatccaaacttggtcaaacttgattatttagaggaagtaaaagtcgtaacaaggtttccgtaggtgaacctgcggaaggatcattattgattttatctatttctgtggatttctggtatattacagcgtcattttatctcaattataactatcaacaacggatctcttggctctcacatcgatgaagaacgcagcgaaccgcgatattttttgtgacttgcagatgtgaatcatcaatctttgaacgcacattgcgcggtatggcattccgtaccgcacggatggaggagcgtgttccctctgggatcgcattgctttcttgaaatggattttttaaactctcaattattacgtcatttcacctccttcatccgagatta

SEQ ID NO: 11 - DNA, 628 bp, Cryptococcus curvatus
cgcccgtcgctactaccgattgaatggcttagtgagatttctggattggcgttaggaagccggcaacggcatcctttggctgagaagttactcaaacttggtcatttagaggaagtaaaagtcgtaacaaggtttccgtaggtgaacctgcggaaggatcattagtgatttgccttcgggctaaactatatccataacacctgtgaactgttgattgacttcggtcaatatttttacaaacattgtgtaatgaacgtcatgttataataacaaatataactttcaacaacggatctcttggctctcgcatcgatgaagaacgcagcgaaatgcgataagtaatgtgaattgcagaattcagtgaatcatcgaatctttgaacgcaacttgcgctctctggtattccggagagcatgcctgtttgagtgtcatgaaatctcaaccattagggtttcttaatggcttggatttggacgtttgccagtcaaatggctcgtcttaaaagagttagtgaatttaacatttgtcttctggcgtaataagtttcgctgggctgatagtgtgaagtttgcttctaatcgtccgcaaggacaattcttgaactctggcctcaaatcaggtaggacta

SEQ ID NO: 12 - DNA, 628 bp, Rhodotorula mucilaginosa; identical to SEQ ID NO: 11

SEQ ID NO: 13 - DNA, 628 bp, Trichosporon sp.
cgcccgtcgctactaccgattgaatggcttagtgagaccctcggattggcgttaagaagccggcaacggcatcttttggccgagaagttggtcaaacttggtcatttagaggaagtaaaagtcgtaacaaggtttccgtaggtgaacctgcggaaggatcattagtgaattgctctttgagcgttaaactatatccatctacacctgtgaactgttgattgacttcggtcaattacttttacaaacattgtgtaatgaacgtcatgttattataacaaaaataactttcaacaacggatctcttggctctcgcatcgatgaagaacgcagcgaaatgcgataagtaatgtgaattgcagaattcagtgaatcatcgaatctttgaacgcaacttgcgctctctggtattccggagagcatgcctgtttgagtatcatgaaatctcaaccattagggtttcttaatggcttggatttgggcgctgccacttgcctggctcgccttaaaagagttagcgtattaacttgtcgatctggcgtaataagtttcgctggtgtagacttgagaagtgcgcttctaatcgtcctcggacaattcttgaactctggtctcaaatcaggtaggacta

SEQ ID NO: 14 - DNA, 716 bp, Sporobolomyces alborubescens
cgcccgtcgctactaccgattgaatggcttagtgaggcctccggattggctattgggagctcgcgagagcacccgactgccgagaagttgtacgaacttggtcatttagaggaagtaaaagtcgtaacaaggtttccgtaggtgaacctgcggaaggatcattagtgaatataggacgtccaacttaacttggagtccgaactctcactttctaaccctgtgcacttgtttgggatagtaactctcgcaagagagcgaactcctattcacttataaacacaaagtctatgaatgtattaaattttataacaaaataaaactttcaacaacggatctcttggctctcgcatcgatgaagaacgcagcgaaatgcgataagtaatgtgaattgcagaattcagtgaatcatcgaatctttgaacgcaccttgcgctccatggtattccgtggagcatgcctgtttgagtgtcatgaatacttcaaccctcctctttcttaatgattgaagaggtgtttggtttctgagcgctgctggcctttacggtctagctcgttcgtaatgcattagcatccgcaatcgaatttcggattgacttggcgtaatagactattcgctgaggaattctagtcttcggattagagccgggttgggttaaaggaagcttctaatcagaatgtctacattttaagattagatctcaaatcaggtaggacta

SEQ ID NO: 15 - DNA, 718 bp, Rhodotorula glutinis
cgcccgtcgctactaccgattgaatggcttagtgaggcctccggattggctattgggagctcgcgagagcacccgactgccgagaagttgtacgaacttggtcatttagaggaagtaaaagtcgtaacaaggtttccgtaggtgaacctgcggaaggatcattagtgaatctaggacgtccaacttaacttggagtccgaactctcactttctaaccctgtgcatctgttttaaaattggctagtagctcttcggagcgaaccaccatttttcacttatacaaacacaaagtctatgaatgtaaacaaatttataacaaaacaaaactttcaacaacggatctcttggctctcgcatcgatgaagaacgcagcgaaatgcgatacgtaatgtgaattgcagaattcagtgaatcatcgaatctttgaacgcaccttgcgctccttggtattccgaggagcatgcctgtttgagtgtcatgaaatcttcaacccacctctttcttagtgaatctggtggtgcttggtttctgagcgctgctctgcttcggcttagctcgttcgtaatgcattagcatccgcaaccgaaacttcggattgacttggcgtaatagactattcgctgaggattccagacttgttctggagccgagttgggttaaaggaagcttctaatcctaaagtctattttttgattagatctcaaatcaggtaggacta

SEQ ID NO: 16 - DNA, 693 bp, Rhodotorula glutinis
cgcccgtcgctactaccgattgaatggcttagtgagggctccggattggcttctgggagccggcaacggcacctagtcgctgagaagttggacgaacttggtcatttagaggaagtaaaagtcgtaacaaggtttccgtaggtgaacctgcggaaggatcattaatgaaatgcaaggacgctctttttagaggtccgacccaattcattttctcacactgtgcacacactactttttacaccatttttaacacttgaagtctaagaatgtaaacagtctcttaattgagcataaaatttaaacaaaactttcagcaacggatctcttggctctcccatcgatgaagaacgcagcgaaatgcgatacgtaatgtgaattgcagaattcagtgaatcatcgaatctttgaacgcaccttgcactctttggtattccgaagagtatgtctgtttgagtgtcatgaaactctcaacccccctgttttgtaatgaaccaggcgtgggcttggattatggctgctgccggcgtaattgtcgactcggctgaaatacacgagctacccatttcataagaaatagacggtttgactcggcgtaataacatatttcgctgaggacgtcacattctttacctagtggtgcttctaatgcgacatctaaactttaagctttagacctcaaatcagtcaggacta

SEQ ID NO: 17 - DNA, 640 bp, Trichosporon behrend
cgcccgtcgctactaccgattgaatggcttagtgagaccctcggattggcgttaggaagccggcaacggcatcctttggccgagaagttggtcaaacttggtcatttagaggaagtaaaagtcgtaacaaggtttccgtaggtgaacctgcggaaggatcattagtgattgccttcataggcttaaactatatccacatacacctgtgaactgttccaccacttgacgcaagtcgagtgtttttacaaacaatgtgtaatgaacgtcgttttattataacaaaataaaactttcaacaacggatctcttggctctcgcatcgatgaagaacgcagcgaattgcgataagtaatgtgaattgcagaattcagtgaatcatcgaatctttgaacgcagcttgcgctctctggtattccggagagcatgcctgtttcagtgtcatgaaatctcaaccactagggtttcctaatggattggatttgggcgtctgcgatctctgatcgctcgccttaaaagagttagcaagtttgacattaatgtctggtgtaataagtttcactgggtccattgtgttgaagcgtgcttctaatcgtccgcaaggacaattactttgactctggcctgaaatcaggtaggacta

SEQ ID NO: 18 - DNA, 476 bp, Geotrichum histeridarum
cgcccgtcgctactaccgatcgaatggcttagtgaggcttccggattgatttgggagagagggcgacttttttcctggaacgagaagctagtcaaacttggtcatttagaggaagtaaaagtcgtaacaaggtttccgtaggtgaacctgcggaaggatcattagaaaaatgcgatattagtggtttattttgctcgccgaaaggcaaacttttaacatacctacctttttttaactataaaaacttttaacaacggatctcttggttctcgcatcgatgaagaacgcagcgaattgcgatacgttttgtgaattgcagaagtgaatcatcaatctttgaacgcacattgcgcctggtggtattccgccaggcatacctgtttgagcgttgttctctctgggattgtctactttcctcaaagaaattaaacaaacaagtttgacacaacacctcaacctcagatcaggtaggacta

SEQ ID NO: 19 - DNA, 687 bp, Rhodotorula aurantiaca
cgcccgtcgctactaccgattgaatggcttagtgaggccttcggattggcttctgggagccggcaacggcacctagtcgctgagaagtttgacgaacttggtcatttagaggaagtaaaagtcgtaacaaggtttccgtaggtgaacctgcggaaggatcattaatgaattttaggacgttctttttagaagtccgaccctttcattttcttacactgtgcacacacttcttttttacacacacttttaacaccttagtataagaatgtaatagtctcttaattgagcataaataaaaacaaaactttcagcaacggatctcttggctctcgcatcgatgaagaacgcagcgaattgcgataagtaatgtgaattgcagaattcagtgaatcatcgaatctttgaacgcaccttgcactctttggtattccgaagagtatgtctgtttgagtgtcatgaaactctcaacccccctattttgtaatgagatgggtgtgggcttggattatggttgtctgtcggcgtaattgccggctcaactgaaatacacgagcaaccctattgaaataaacggtttgacttggcgtaataattatttcgctaaggacgctttcttcaaatataagaggtgcttctaattcgcttctaatagcatttaagctttagacctcaaatcagtcaggacta

SEQ ID NO: 20 - DNA, 687 bp, Cryptococcus curvatus; identical to SEQ ID NO: 19

SEQ ID NO: 21 - DNA, 636 bp, Trichosporon domesticum
cgcccgtcgctactaccgattgaatggcttagtgagacctccggattggcgttgagaagccggcaacggcatctcttggctgagaagttggtcaaacttggtcatttagaggaagtaaaagtcgtaacaaggtttccgtaggtgaacctgcggaaggatcattagtgattgccttaattggcttaaactatatccatctacacctgtgaactgtttgattgaatcttcggattcgattttatacaaacattgtgtaatgaacgtcattatattataacaaaaaaaaaactttcaacaacggatctcttggctctcgcatcgatgaagaacgcagcgaaatgcgataagtaatgtgaattgcagaattcagtgaatcatcgaatctttgaacgcaacttgcgctctctggtattccggagagcatgcctgtttgagtgtcatgaaatctcaaccattagggtttcttaatggcttggatttggaggtttgccagtctgactggctcctcttaaaagagttagcaagttgaactattgctatctggcgtaataagtttcgctggaatggtattgtgaagcgtgcttctaatcgtcttcggacaattttttgactctggcctcaaatcaggtaggacta

SEQ ID NO: 22 - DNA, 711 bp, Rhodotorula toruloides
cgcccgtcgctactaccgattgaatggcttagtgaggcctccggattggctatcgggagctcgcgagagcacctgactgccgagaagttgtacgaacttggtcatttagaggaagtaaaagtcgtaacaaggtttccgtaggtgaacctgcggaaggatcattagtgaatattagggtgtccaacttaacttggagcccgaccctcactttctaaccctgtgcatttgtcttgggtagtagctcgtgtcagcgagcgaatcccatttcacttacaaacacaaagtctatgaatgtaacaaatttataacaaacaaaactttcaacaacggatctcttggctctcgcatcgatgaagaacgcagcgaaatgcgatacgtaatgtgaattgcagaattcagtgaatcatcgaatctttgaacgcaccttgcgctccatggtattccgtggagcatgcctgtttgagtgtcatgaattcttcaacccacctctttcttagtgaatcaggcggtgtttggattctgagcgttgctggcttcgcggcctagctcgctcgtaatgcattagcatccgcaatcgaacttcggattgactcggcgtaatagactattcgctgaggattctggtctctgactggagccgggtaagattaaaggaagctactaatcctcatgtctatcttttgagattagacctcaaatcaggtaggacta

SEQ ID NO: 23 - DNA, 753 bp, Rhodotorula terpendoidalis
cgcccgtcgctactaccgattgaatggcttagtgaggcctccggactggctattgggatctcgcgagagaacctgactgctgggaagttgtacgaacttggtcatttagaggaagtaaaagtcgtaacaaggtttccgtaggtgaacctgcggaaggatcattaatgaatattagggtgctcttttcatcaaagaggcctgaccttcattcttctaccctgtgcactattcaaacattcggcagttggtaatttggcttgtaaaagagccagacgactctgctgaattcactcttaaactctaaagtataagaatgttacaaataaaacaaataaaactttcaacaacggatctcttggctctcgcatcgatgaagaacgcagcgaaatgcgataagtaatgtgaattgcagaattcagtgaatcatcgaatctttgaacgcaccttgcgctcgctggtattccggcgagcatgcctgtttgagtgtcatgaaaacctcaacccttcaattccttgttgaattgtaaggtgtttggattctgaatgtttgctggcttgaagggcccttggctacttcaaaagcgaagctcattcgtaatacattagcatctcaatttcgaatattcggattgactcggcgtaatagactttattcgctgaggacaccttcacaaggtggccgaatttcgaggtagaagcttccaattcgatcaaaagtcactcttagtttagacctcagatcaggcaggacta

SEQ ID NO: 24 - DNA, 456 bp, Yarrowia lipolytica
cgcccgtcgctactaccgattgaatggtttagtgagaccttgggagggcgagatgaggggggcaaccccttttgaacatccaaacttggtcaaacttgattatttagaggaagtaaaagtcgtaacaaggtttccgtaggtgaacctgcggaaggatcattattgattttatctatttctgtggatttctggtatattacagcgtcattttatctcaattataactatcaacaacggatctcttggctctcacatcgatgaagaacgcagcgaaccgcgatattttttgtgacttgcagatgtgaatcatcaatctttgaacgcacattgcgcggtatggtattccgtaccgcacggatggaggagcgtgttccctctgggatcgcattgctttcttgaaatggattttttaaactctcaattattacgtcatttcacctccttcatccgagatta

SEQ ID NO: 25 - DNA, 709 bp, Rhodotorula glutinis
cgcccgtcgctactaccgattgaatggcttagtgaggcctccggattggctattgggagctcgcgagagcacctgactgccgagaagttgtacgaacttggtcatttagaggaagtaaaagtcgtaacaaggtttccgtaggtgaacctgcggaaggatcattagtgaatattagggtgtccaacttaacttggaacccgaccctcactttctaaccctgtgcatttgtcttgggtagtagcttgcgtcggcgagcgaatcccatttcacttacaaacacaaagtctatgaatgtaacaaatttataacaaacaaaactttcaacaacggatctcttggctctcgcatcgatgaagaacgcagcgaaatgcgatacgtaatgtgaattgcagaattcagtgaatcatcgaatctttgaacgcaccttgcgctccatggtattccgtggagcatgcctgtttgagtgtcatgaattcttcaacccacctatttcttagtgaatcaggcggtgtttggattctgagcgctgctggcctcacggcctagctcgctcgtaatgcattagcatccgcaatcgaacttcggattgactcggcgtaatagactattcgctgaggattctggtctctgactggagccgggtgagattaaaggaagctactaatcctcatgtctatcttgagattagacctcaaatcaggtaggacta

SEQ ID NO: 26 - DNA, 500 bp, Yarrowia lipolytica
gtccctgccctttgtacacaccgcccgtcgctactaccgattgaatggtttagtgagaccttgggagggcgagatgaggggggcaaccccttctgaacatccaaacttggtcaaacttgattatttagaggaagtaaaagtcgtaacaaggtttccgtaggtgaacctgcggaaggatcattattgattttatctatttctgtggatttctattctattacagcgtcattttatctcaattataactatcaacaacggatctcttggctctcacatcgatgaagaacgcagcgaaccgcgatattttttgtgacttgcagatgtgaatcatcaatctttgaacgcacattgcgcggtatggcattccgtaccgcacggatggaggagcgtgttccctctgggatcgcattgctttcttgaaatggattttttaaactctcaattattacgtcatttcacctccttcatccgagattacccgctgaacttaagcatatcaa

SEQ ID NO: 27 - DNA, 707 bp, Lipomyces tetrasporus
cgcccgtcgctactaccgattgaatggcttagtgaggcctccggattggctattgggagctcgcgagagcacctgactgctgagaagttgtacgaacttggtcatttagaggaagtaaaagtcgtaacaaggtttccgtaggtgaacctgcggaaggatcattagtgaatctaggacgtccaacttaacttggagtccgaaatctcactttctaaccctgtgcatctgttaattggaatagtagctcttcggagtgaaccaccattcacttataaaacacaaagtctatgaatgtatacaaatttataacaaaacaaaactttcaacaacggatctcttggctctcgcatcgatgaagaacgcagcgaaatgcgatacgtaatgtgaattgcagaattcagtgaatcatcgaatctttgaacgcaccttgcgctccttggtattccgaggagcatgcctgtttgagtgtcatgaaatcttcaacccacctctttcttagtgaatctggtggtgcttggtttctgagcgctgctctgcttcggcttagctcgttcgtaatgcattagcatccgcaaccgaacttcggattgacttggcgtaatagactattcgctgaggattctagtttactagagccgagttgggttaaaggaagctcctaatcctaaagtctattttttgattagatctcaaatcaggtaggacta

SEQ ID NO: 28 - DNA, 457 bp, Yarrowia lipolytica
cgcccgtcgctactaccgattgaatggtttagtgagaccttgggagggcgagatgaggggggcaaccccttctgaacatccaaacttggtcaaacttgattatttagaggaagtaaaagtcgtaacaaggtttccgtaggtgaacctgcggaaggatcattattgattttatctatttctgtggatttctattctattacagcgtcattttatctcaattataactatcaacaacggatctcttggctctcacatcgatgaagaacgcagcgaaccgcgatattttttgtgacttgcagatgtgaatcatcaatctttgaacgcacattgcgcggtatggcattccgtaccgcacggatggaggagcgtgttccctctgggatcgcattgctttcttgaaatggatttttttaaactctcaattattacgtcatttcacctccttcatccgagatta

SEQ ID NO: 29 - DNA, 1022 bp, Lipomyces tetrasporus
cgcccgtcgctactaccgattgaatggcttagtgaggccttcggactggctccagaaaatgggaaaccattatcaggagctggaaagttggtcaaacttggtcatttagaggaagtaaaagtcgtaacaaggtttccttccgtagcacttactgaagctttagcagcccgaaaaggcgaatgctagcgactataaataaatatggcgttcttaaatgctagtctctgattagaggcgacattgccaaattgcggggacatcctaaagatcttgataccaagctggtagtcgaaagacgccagtggccgagctaacagccctgggtatggtaataattcaagatatggaacaatgggtaatccgcagccaagtcctaaactacgcaagtagcatggatgcagttcacaggccaaatggtgatgggtagattactaaatctgcttaagatatggtcggtcccgctgtgagagcagatgggaagctacaaagcagactcgtgagtttgcgcaaacgtaactaaaaacgttccgtaggtgaacctgcggaaggatcattactgagtatttgtcttttaaagacatctctctatccataaactcttttttctaaaaagacatgatttacacaattagtctgaatgattatataaaaatcttcaaaactttcaacaacggatctcttggttctcgcatcgatgaagaacgcagcaaaatgcgataagtattgtgaattgcaggattttgtgaatcatcgaatttttgaacgcacattgcaccttctggtattccggagggtatacctgtttgagcgtcatttatatactcaaaacttcgttttggtgatgggcacatatctggtgagagctagatttgcctgaaatatagtggtagagattgctacgagttatgcaagttagccaatgctattaagttaattcgttggtgaagcatgcggagctttagtgatcgccttccttaactattggaatttttctaattttgacctcaaatcaggcaggagta

SEQ ID NO: 30 - DNA, 712 bp, Rhodosporidium sphaerocarpum
cgcccgtcgctactaccgattgaatggcttagtgaggcctccggaccggctattgggagctcgcgagagcacccgactgctgggaagttgtacgaacttggtcatttagaggaagtaaaagtcgtaacaaggtttccgtaggtgaacctgcggaaggatcattagtgaatataggacgtccaacttaacttggagtccgaactctcactttctaaccctgtgcatttgtttgggatagtagcctctcggggtgaactcctattcactcataaacacaaagtctatgaatgtatttaatttataacaaaataaaactttcaacaacggatctcttggctctcgcatcgatgaagaacgcagcgaaatgcgataagtaatgtgaattgcagaattcagtgaatcatcgaatctttgaacgcaccttgcgctccatggtattccgtggagcatgcctgtttgagtgtcatgaatacttcaaccctcctcttttctagtgaaagagaaggtgcttggtttctgagcgttttgctggcctcacggtcgagctcgctcgtaatgcattagcatccgcaatcgaacttcggattgacttggcgtaatagactattcgctgaggaattctaatcttcggattagagccgggttgggttaaaggaagcttctaatcctaatgtctatatttttagattagatctcaaatcaggtaggacta

SEQ ID NO: 31 - DNA, 631 bp, Trichosporon brassicae
cgcccgtcgctactaccgattgaatggcttagtgagacctccggattggcgttgagaagccggcaacggcatctcttggccgagaagttggtcaaacttggtcatttagaggaagtaaaagtcgtaacaaggtttccgtaggtgaacctgcggaaggatcattagtgattgccttaattggcttaaactatatccaactacacctgtgaactgttcgattgaatcttcgattcaattttacaaacattgtgtaaagaacgtcattagatcataacaaaaaaaaacttttaacaacggatctcttggctctcgcatcgatgaagaacgcagcgaaatgcgataagtaatgtgaattgcagaattcagtgaatcatcgaatctttgaacgcaacttgcgctctctggtattccggagagcatgcctgtttgagtgtcatgaaatctcacacatcaaggtttcttgatgaagtggatttggaggttgccagtctaactggctcctcttaaaggagttagcatatttgattattgctgtctggcgtaataagtttcgctagtttggcattttgaagtgtgcttctaatcgtcttcggacaattttttgactctggcctcaaatcaggtaggacta

SEQ ID NO: 32 - DNA, 627 bp, Cryptococcus curvatus
cgcccgtcgctactaccgattgaatggcttagtgagatttccggattggcgttaggaagccggcaacggcatcctttggctgagaagctactcaaacttggtcatttagaggaagtaaaagtcgtaacaaggtttccgtaggtgaacctgcggaaggatcattagtgatttgccttcgggctaactatatccataacacctgtgaactgttgattgacttcggtcaatatttttacaaacattgtgtaatgaacgtcatgttataataacaaatataactttcaacaacggatctcttggctctcgcatcgatgaagaacgcagcgaaatgcgataagtaatgtgaattgcagaattcagtgaatcatcgaatctttgaacgcaacttgcgctctctggtattccggagagcatgcctgtttgagtgtcatgaaatctcaaccattagggtttcttaatggcttggatttggacgtttgccagtcaaatggctcgtcttaaaagagttagtgaatttaacatttgtcttctggcgtaataagtttcgctgggctgatagtgtgaagtttgcttctaatcgtccgcaaggacaattcttgaactctggcctcaaatcaggtaggacta

SEQ ID NO: 33 - DNA, 627 bp, Lipomyces tetrasporus; identical to SEQ ID NO: 32

SEQ ID NO: 34 - DNA, 637 bp, Lipomyces starkeyi
cgcccgtcgctactaccgattgaatggcttagtgaggccttcggactggctccagaaaatgggaaaccattatcaggagctggaaagttggtcaaacttggtcatttagaggaagtaaaagtcgtaacaaggtttccgtaggtgaacctgcggaaggatcattactgagtatttgtcttttcaagacatctctctatccataaactcttttttttaaaaagacatgatttataacaattagtctgaatgattatttttaaatcttcaaaactttcaacaacggatctcttggttctcgcatcgatgaagaacgcagcaaattgcgataagtaatgtgaattgcaggattttgtgaatcatcgaatttttgaacgcacattgcaccttctggtattccggagggtatacctgtttgagcgtcatttatatactcaaaacttacgttttggtgatgggcacgtatctggcttctaagttagatttgcctgaaatatagcggtagaggtcgctagaagcgatgcaagttagccaatgctattaaagttaattcgttggtgacgcatgttgagcttttggtgaagtcttccttaattattggaatttttttctaattttgacctcaaatcaggcaggagta

SEQ ID NO: 35 - DNA, 457 bp, Yarrowia lipolytica
cgcccgtcgctactaccgattgaatggtttagtgagaccttgggagggcgagatgaggggggcaaccccttttgaacatccaaacttggtcaaacttgattatttagaggaagtaaaagtcgtaacaaggtttccgtaggtgaacctgcggaaggatcattattgattttatctatttctgtggatttctattctattacagcgtcattttatctcaattataactatcaacaacggatctcttggctctcacatcgatgaagaacgcagcgaaccgcgatattttttgtgacttgcagatgtgaatcatcaatctttgaacgcacattgcgcggtatggcattccgtaccgcacggatggaggagcgtgttccctctgggatcgcattgctttcttgaaatggatttttttaaactctcaattattacgtcatttcacctccttcatccgagatta

SEQ ID NO: 36 - DNA, 631 bp, Trichosporon loubieri
cgcccgtcgctactaccgattgaatggcttagtgagacctccggattggcgttgagaagccggcaacggcatctcttggccgagaagttggtcaaacttggtcatttagaggaagtaaaagtcgtaacaaggtttccgtaggtgaacctgcggaaggatcattagtgattgccatcttggcttaaactatatccatctacacctgtgaaccgtttgattgaatcttctgattcaattttacaaacattgtgtaatgaacgtcattagatcataataagaaaaaactttcaacaacggatctcttggctctcgcatcgatgaagaacgcagcgaaatgcgataagtaatgtgaattgcagaattcagtgaatcatcgaatctttgaacgcaacttgcgctctctggtattccggagagcatgcctgtttgagtgtcatgaaatctcaaccattagggtttcttaatggcttggatttggaggttgccattctaaatggctcctcttaaaagagttagcgagtttaactattgctatctggcgtaataagtttcgctggaatggtattgtgaagcgcgcttctaatcgtcttcggacaattttttgactctggcctcaaatcaggtaggacta

SEQ ID NO: 37 - DNA, 476 bp, Geotrichum vulgare
cgcccgtcgctactaccgattgaatggcttagtgaggcttccggattgattagttggagagggagacttttctgactgaacgagaagctagtcaaacttggtcatttagaggaagtaaaagtcgtaacaaggtttccgtaggtgaacctgcggaaggatcattaaagatttaatattaattgtgaaattaaaacgatattaacaaaaaatcatacaatcaattataaaaaaaatcaaaacttttaacaatggatctcttggttctcgtatcgatgaagaacgcagcgaaacgcgatatttcttgtgaattgcagaagtgaatcatcagtttttgaacgcacattgcactttggggtatcccccaaagtatacttgtttgagcgttgtttctctcttggaattgcattgcttttctaaaaaatcgaatcaaattcgtttgaaacatccattcttcaacctcagatcaagtaggatta

SEQ ID NO: 38 - DNA, 710 bp, Rhodosporidium toruloides
cgcccgtcgctactaccgattgaatggcttagtgaggcctccggattggctattgggagctcgcgagagcacctgactgccgagaagttgtacgaacttggtcatttagaggaagtaaaagtcgtaacaaggtttccgtaggtgaacctgcggaaggatcattagtgaatattagggtgtccaacttaacttggagcccgaccctcactttctaaccctgtgcatttgtcttgggtagtagcttgcgtcagcgagcgaatcccatttcacttacaaacacaaagtctatgaatgtaacaaatttataacaaaacaaaactttcaacaacggatctcttggctctcgcatcgatgaagaacgcagcgaaatgcgatacgtaatgtgaattgcagaattcagtgaatcatcgaatctttgaacgcaccttgcgctccatggtattccgtggagcatgcctgtttgagtgtcatgaattcttcaacccacctctttcttagtgaatcaggcggtgtttggattctgagcgctgctggcttcgcggcctagctcgctcgtaatgcattagcatccgcaatcgaacttcggattgactcggcgtaatagactattcgctgaggattctggtctctgactggagccgggtaaggttaaagggagctactaatcctcatgtctatcttgagattagacctcaaatcaggtaggacta

SEQ ID NO: 39 - DNA, 707 bp, Rhodotorula glutinis
cgcccgtcgctactaccgattgaatggcttagtgaggcctccggattggctattgggagctcgcgagagcacctgactgctgagaagttgtacgaacttggtcatttagaggaagtaaaagtcgtaacaaggtttccgtaggtgaacctgcggaaggatcattagtgaatctaggacgtccaacttaacttggagtccgaactctcactttctaaccctgtgcatctgttaattggaatagtagctcttcggagtgaaccaccattcacttataaaacacaaagtctatgaatgtatacaaatttataacaaaacaaaactttcaacaacggatctcttggctctcgcatcgatgaagaacgcagcgaaatgcgatacgtaatgtgaattgcagaattcagtgaatcatcgaatctttgaacgcaccttgcgctccttggtattccgaggagcatgcctgtttgagtgtcatgaaatcttcaacccacctctttcttagtgaatctggtggtgcttggtttctgagcgctgctctgcttcggcttagctcgttcgtaatgcattagcatccgcaaccgaacttcggattgacttggcgtaatagactattcgctgaggattctagtttactagagccgagttgggttaaaggaagctcctaatcctaaagtctattttttgattagatctcaaatcaggtaggacta

SEQ ID NO: 40 - DNA, 707 bp, Lipomyces orientalis; identical to SEQ ID NO: 39

SEQ ID NO: 41 - DNA, 627 bp, Rhodotorula aurantiaca
cgcccgtcgctactaccgattgaatggcttagtgagatttccggattggcgttaggaagccggcaacggcatcctttggctgagaagctactcaaacttggtcatttaaaggaagtaaaagtcgtaacaaggtttccgtaggtgaacctgcggaaggatcattagtgatttgccttcgggctaactatatccataacacctgtgaactgttgattgacttcggtcaatatttttacaaacattgtgtaatgaacgtcatgttataataacaaatataactttcaacaacggatctcttggctctcgcatcgatgaagaacgcagcgaaatgcgataagtaatgtgaattgcagaattcagtgaatcatcgaatctttgaacgcaacttgcgctctctggtattccggagagcatgcctgtttgagtgtcatgaaatctcaaccattagggtttcttaatggcttggatttggacgtttgccagtcaaatggctcgtcttaaaagagttagtgaatttaacatttgtcttctggcgtaataagtttcgctgggctgatagtgtgaagtttgcttctaatcgtccgcaaggacaattcttgaactctggcctcaaatcaggtaggacta

SEQ ID NO: 42 - DNA, 899 bp, Torulaspora delbrueckii
cgcccgtcgctagtaccgattgaatggcttagtgaggcctcaggatctgcttagagaagggggcaactccatctcagagcggagaatctggtcaaacttggtcatttagaggaactaaaagtcgtaacaaggtttccgtaggtgaacctgcggaaggatcattagagaaatctatatgaatgaagttagaggacgtctaaagatactgtaagagaggatcaggttcaagaccagcgcttaattgcgcggttgcggcttggttcgccttttgcggaacatgtcttttctcgttgttaactctacttcaacttctacaacactgtggagttttctacacaacttttcttctttgggaagatacgtcttgtgcgtgcttcccagaggtgacaaacacaaacaactttttattattataaaccagtcaaaaccaatttcgttatgaaattaaaaatatttaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacaatcatgtttggtagtgagtgatactctgtcaagggttaacttgaaattgctagcctgttatttggttgtgattttgctggcttggatgactttgtccagtctagctaataccgaattgtcgtattaggttttaccaacttcggcagactgtgtgttggctcgggcgctttaaagactttgtcgtaaacgatttatcgtttgtttgagcttttcgcatacgcaatccgggcgaacaatactctcaaagtttgacctcaaatcaggtaggaata

(19) United States
(12) Patent Application Publication
(10) Pub. No.: US 2011/0252696 A1
(43) Pub. Date: Oct. 20, 2011

(54) Fuel And Chemical Production From Oleaginous Yeast

(75) Inventors: Scott Franklin, San Diego, CA (US); Stephen M. Decker, San Francisco, CA (US); Janice Wee, San Mateo, CA (US)

(73) Assignee: Solazyme, Inc., South San Francisco, CA (US)

(21) Appl. No.: 13/087,311

(22) Filed: Apr. 14, 2011

Related U.S. Application Data
(60) Provisional application No. 61/324,286, filed on Apr. 14, 2010.
(60) Provisional application No. 61/333,718, filed on May 11, 2010.
(60) Provisional application No. 61/365,235, filed on Jul. 16, 2010.

(51) Int. Cl. C10L 1/19; C12P 7/64

(52) U.S. Cl. 44/388; 435/134

(57) ABSTRACT

Oleaginous yeast can be used to produce oil that can be extracted from oleaginous yeast biomass and converted into a wide variety of useful products, including fuels, hydrocarbon compositions, and/or oleochemicals.

### CROSS-REFERENCE TO RELATED APPLICATIONS

This application claims the benefit under 35 U.S.C. 119(e) of U.S. Provisional Patent Application No. 61/324,286, filed Apr. 14, 2010, U.S. Provisional Patent Application No. 61/333,718, filed May 11, 2010, and U.S. Provisional Patent Application No. 61/365,235, filed Jul. 16, 2010. Each of these applications is incorporated herein by reference in its entirety for all purposes.

### REFERENCE TO A SEQUENCE LISTING

This application includes a sequence listing, appended hereto as pages 1-14.

### FIELD OF THE INVENTION

The present invention relates to the production of oils, fuels, and oleochemicals made from oleaginous yeast.

### BACKGROUND OF THE INVENTION

Fossil fuel is a general term for buried combustible geologic deposits of organic materials, formed from decayed plants and animals that have been converted to crude oil, coal, natural gas, or heavy oils by exposure to heat and pressure in the earth's crust over hundreds of millions of years. Fossil fuels are a finite, non-renewable resource.

Increased demand for energy by the global economy has also placed increasing pressure on the cost of hydrocarbons. Aside from energy, many industries, including plastics and chemical manufacturers, rely heavily on the availability of hydrocarbons as a feedstock for their manufacturing processes. Cost-effective alternatives to current sources of supply could help mitigate the upward pressure on energy and these raw material costs.

PCT Pub. No. WO 2008/151149 describes methods and materials for cultivating microbes, including oleaginous yeast, for the production of oil and particularly exemplifies the production of diesel fuel from oil produced by the microalgae Chlorella protothecoides. There remains a need for improved methods for producing oil in oleaginous yeast, particularly for methods that produce oils with shorter chain length and a higher degree of saturation and without pigments, with greater yield and efficiency. The present invention meets this need.

### SUMMARY OF THE INVENTION

Oleaginous yeast can be used to produce oil that can be extracted from oleaginous yeast biomass and converted into a wide variety of useful products, including fuels, hydrocarbon compositions, and/or oleochemicals.

The present invention is directed to a method for producing oil by (a) culturing an oleaginous yeast in a culture medium under conditions such that oleaginous yeast biomass comprising at least about 20% oil by dry weight is produced, and (b) extracting the oil from the biomass. In some cases, the biomass comprises at least about 40% oil by dry weight.

In some embodiments, the oleaginous yeast is a strain of a genus selected from the group consisting of Candida, Cryptococcus, Debaromyces, Endomycopsis, Geotrichum, Hyphopichia, Lipomyces, Lypomyces, Pichia, Rodosporidium, Rhodotorula, Sporobolomyces, Starmerella, Torulaspora, Trichosporon, Wickerhamomyces, Yarrowia, and Zygoascus. In some cases, the strain is selected from the group consisting of Candida apicola, Cryptococcus curvatus, Cryptococcus terricolus, Debaromyces hansenii, Endomycopsis vernalis, Geotrichum carabidarum, Geotrichum cucujoidarum, Geotrichum histeridarum, Geotrichum silvicola, Geotrichum vulgare, Hyphopichia burtonii, Lipomyces lipofer, Lypomyces orentalis, Lipomyces starkeyi, Lipomyces tetrasporous, Pichia mexicana, Rodosporidium sphaerocarpum, Rhodosporidium toruloides, Rhodotorula aurantiaca, Rhodotorula dairenensis, Rhodotorula diffluens, Rhodotorula glutinus, Rhodotorula glutinis var. glutinis, Rhodotorula gracilis, Rhodotorula graminis, Rhodotorula minuta, Rhodotorula mucilaginosa, Rhodotorula mucilaginosa var. mucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri var. loubieri, Trichosporon montevideense, Trichosporon pullulans, Trichosporon sp., Wickerhamomyces canadensis, Yarrowia lipolytica, Zygoascus meyerae, and any strain that has a 3′ region of fungal 18S and/or 5′ region of fungal 26S rRNA that is at least 80% identical to a corresponding region from any of the foregoing strains.

In some embodiments, the oleaginous yeast strain has a 3′ region of fungal 18S and/or 5′ region of fungal 26S rRNA that is at least 95% identical to a corresponding region from any of the strains discussed herein.

In some embodiments, the oleaginous yeast are cultured in a medium comprising a carbon source selected from the group consisting of glucose, fructose, sucrose, lactose, galactose, xylose, mannose, rhamnose, arabinose, glycerol, and acetate. In some cases, the yeast are cultured in a medium comprising a carbon source selected from the group consisting of mixtures of glycerol and glucose, mixtures of glucose and xylose, mixtures of fructose and glucose, mixtures of sucrose and depolymerized sugar beet pulp, black liquor, corn starch, depolymerized cellulosic material, corn stover, sugar beet pulp, switchgrass, milk whey, molasses, potato, rice, sorghum, sugar cane, and wheat, or a combination of two or more of the group. In another embodiment, the oleaginous yeast are cultured in a culture medium comprising a carbon source selected from the group consisting of depolymerized sugar beet pulp, black liquor, corn starch, depolymerized cellulosic material, corn stover, sugar beet pulp, switchgrass, milk whey, molasses, potato, rice, sorghum, sugar cane, thick cane juice, sugar beet juice, and wheat.

In some embodiments, the oil is extracted by solvent extraction or by using an expeller press. In some embodiments, the oil is extracted from the biomass by solvent extraction. In some cases, extraction is performed by applying pressure to the biomass using an expeller press.

In some embodiments, the method further comprises converting the oil to a fuel. In some cases, the fuel is selected from the group consisting of biodiesel, renewable diesel, and jet fuel. In some cases, the fuel is renewable diesel and has a T10-T90 of at least 20° C. measured by ASTM D86 distillation.

In some embodiments, the method further comprises subjecting the oil to a chemical reaction. In some cases, the chemical reaction is selected from the group consisting of cracking, esterification, hydrogenation, hydroprocessing, hydrotreating, interesterification, isomerization, saponification, metathesis and transesterification. In some cases, metathesis is performed using the Grubbs catalyst.

In some embodiments, the oleaginous yeast produces oil having a lipid profile comprising an attribute selected from the group consisting of: (a) a lipid profile of below 10% C18:2; (b) a lipid profile of below 50% C18:1 or a lipid profile of at least 50% C18:1; and (c) a lipid profile of at least 10% C16:1 or a lipid profile of at least 40% C16:1. In some embodiments, the oleaginous yeast produces oil having a lipid profile of below 15% C18:2. In some embodiments, the oleaginous yeast produces oil having a lipid profile of below 10% C18:2. In some cases, the oleaginous yeast produces oil having a lipid profile of below 50% C18:1. In some cases, the oleaginous yeast produces oil having a lipid profile of between about 40% to about 60% C18:1. In some cases, the oleaginous yeast produces oil having a lipid profile of at least 10% C16:1. In some cases, the oleaginous yeast produces oil having a lipid profile of at least 40% C16:1. In some cases, the oleaginous yeast produces oil having a lipid profile of between about 10% to about 45% C18:1. In some embodiments, the biomass comprises over 50% oil by dry weight. In one aspect, the present invention is directed to an oil isolated from oleaginous yeast, preferably an oil comprising at least 10% C16:1.

These and other aspects and embodiments of the invention are described in the accompanying drawings, a brief description of which immediately follows, and in the detailed description of the invention below, and are exemplified in the examples below. Any or all of the features discussed above and throughout the application can be combined in various embodiments of the present invention.

### BRIEF DESCRIPTION OF THE DRAWINGS

FIG. 1 summarizes the results of a carbon utilization screen with oleaginous yeast species that is described in Example 3.

### DETAILED DESCRIPTION OF THE INVENTION

The present invention arises in part from the discovery that oleaginous yeast and certain species thereof have unexpectedly advantageous properties for the production of oils, fuels, and other hydrocarbon or lipid compositions economically and in large quantities. The oils produced by these microorganisms can be used in the transportation fuel, petrochemical, and/or food and cosmetic industries, among other applications. Transesterification of lipids yields long-chain fatty acid esters useful as biodiesel. Other enzymatic and chemical processes can be tailored to yield fatty acids, aldehydes, alcohols, alkanes, and alkenes. In some applications, renewable diesel, jet fuel, or other hydrocarbon compositions are produced. The present invention also provides methods of cultivating oleaginous yeast for increased productivity and increased lipid yield, and/or for more cost-effective production of the compositions described herein.

This detailed description of the invention is divided into sections for the convenience of the reader. Section I provides definitions of terms used herein. Section II provides a description of oleaginous yeast strains and culture conditions useful in the methods of the invention. Section III describes methods of the invention for extraction of triglyceride oil from oleaginous yeast. Section IV describes methods of the invention for making fuels and chemicals from extracted triglyceride oils. Section V provides examples and embodiments of the invention.

### I. DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Area Percent” refers to the area percent of chromatographic, spectroscopic, and other peaks generated during experimentation. The determination of the area under the curve of a peak and the area percent of a particular peak is routinely accomplished by one of skill in the art. For example, in FAME GC/FID detection methods in which fatty acid molecules in the sample are converted into a fatty acid methyl ester (FAME), a separate peak is observed for a fatty acid of 14 carbon atoms with no unsaturation (C14:0) compared to any other fatty acid such as C14:1. The peak area for each class of FAME is directly proportional to its percent composition in the mixture and is calculated based on the sum of all peaks present in the sample (i.e. [area under specific peak/total area of all measured peaks]×100). When referring to lipid profiles of oils and cells of the invention, “at least 4% C8-C14” means that at least 4% of the total fatty acids in the cell or in the extracted glycerolipid composition have a chain length that includes 8, 10, 12 or 14 carbon atoms.

“Axenic” is a culture of an organism free from contamination by other living organisms.

“Biodiesel” is a biologically produced fatty acid alkyl ester suitable for use as a fuel in a diesel engine.

“Biomass” is material produced by growth and/or propagation of cells. Biomass may contain cells and/or intracellular contents as well as extracellular material, includes, but is not limited to, compounds secreted by a cell.

“Bioreactor” and “fermentor” mean an enclosure or partial enclosure, such as a fermentation tank or vessel, in which cells are cultured, typically in suspension.

“Catalyst” is an agent, such as a molecule or macromolecular complex, capable of facilitating or promoting a chemical reaction of a reactant to a product without becoming a part of the product. A catalyst increases the rate of a reaction, after which, the catalyst may act on another reactant to form the product. A catalyst generally lowers the overall activation energy required for the reaction such that it proceeds more quickly or at a lower temperature. Thus, a reaction equilibrium may be more quickly attained using a catalyst. Examples of catalysts include enzymes, which are biological catalysts; heat, which is a non-biological catalyst; and metals used in fossil oil refining processes.

“Cellulosic material” is the product of digestion of cellulose, including glucose and xylose, and optionally additional compounds such as disaccharides, oligosaccharides, lignin, furfurals and other compounds. Nonlimiting examples of sources of cellulosic material include sugar cane bagasses, sugar beet pulp, corn stover, wood chips, sawdust and switchgrass.

“Co-culture”, and variants thereof such as “co-cultivate” and “co-ferment”, refer to the presence of two or more types of cells in the same bioreactor. The two or more types of cells may both be microorganisms, such as oleaginous yeast, or may be an oleaginous yeast cell cultured with a different cell type. The culture conditions may be those that foster growth and/or propagation of the two or more cell types or those that facilitate growth and/or proliferation of one, or a subset, of the two or more cells while maintaining cellular growth for the remainder.

“Cofactor” is any molecule, other than the substrate, required for an enzyme to carry out its enzymatic activity.

“Complementary DNA” or “cDNA” is a DNA copy of mRNA, usually obtained by reverse transcription of messenger RNA (mRNA) or amplification (e.g., via polymerase chain reaction (“PCR”)).

“Cultivated”, and variants thereof such as “cultured” and “fermented”, refer to the intentional fostering of growth (increases in cell size, cellular contents, and/or cellular activity) and/or propagation (increases in cell numbers via mitosis) of one or more cells by use of selected and/or controlled conditions. The combination of both growth and propagation may be termed proliferation. Examples of selected and/or controlled conditions include the use of a defined medium (with known characteristics such as pH, ionic strength, and carbon source), specified temperature, oxygen tension, carbon dioxide levels, and growth in a bioreactor. Cultivate does not refer to the growth or propagation of microorganisms in nature or otherwise without human intervention; for example, natural growth of an organism that ultimately becomes fossilized to produce geological crude oil is not cultivation.

“Cytolysis” is the lysis of cells in a hypotonic environment. Cytolysis is caused by excessive osmosis, or movement of water, towards the inside of a cell (hyperhydration). If the cell cannot withstand the osmotic pressure of the water inside, it bursts.

“Delipidated meal” and “delipidated microbial biomass” is microbial biomass after oil (including lipids) has been extracted or isolated from it, either through the use of mechanical (i.e., exerted by an expeller press) or solvent extraction or both. Delipidated meal has a reduced amount of oil/lipids as compared to before the extraction or isolation of oil/lipids from the microbial biomass but does contain some residual oil/lipid.

“Dry weight” and “dry cell weight” mean weight determined in the relative absence of water. For example, reference to oleaginous yeast biomass as comprising a specified percentage of a particular component by dry weight means that the percentage is calculated based on the weight of the biomass after substantially all water has been removed.

“Exogenous gene” is a nucleic acid that codes for the expression of an RNA and/or protein that has been introduced (“transformed”) into a cell. A transformed cell may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome or as an episomal molecule.

“Exogenously provided” refers to a molecule provided to the culture media of a cell culture.

“Expeller pressing” is a mechanical method for extracting oil from raw materials such as soybeans and rapeseed. An expeller press is a screw type machine, which presses material through a caged barrel-like cavity. Raw materials enter one side of the press and spent cake exits the other side while oil seeps out between the bars in the cage and is collected. The machine uses friction and continuous pressure from the screw drives to move and compress the raw material. The oil seeps through small openings that do not allow solids to pass through. As the raw material is pressed, friction typically causes it to heat up.

“Fixed carbon source” is a molecule(s) containing carbon, typically an organic molecule, that is present at ambient temperature and pressure in solid or liquid form in a culture media that can be utilized by a microorganism cultured therein.

“Glycerolipid profile” means the distribution of different carbon chain lengths and saturation levels of glycerolipids in a particular sample of biomass or oil. For example, a sample could have a glycerolipid profile in which approximately 60% of the glycerolipid is C18:1, 20% is C18:0, 15% is C16:0, and 5% is C14:0. When a carbon length is referenced generically, such as “C:18”, such reference can include any amount of saturation; for example, oleaginous yeast biomass that contains 20% (by weight/mass) lipid as C:18 can include C18:0, C18:1, C18:2, and the like, in equal or varying amounts, the sum of which constitute 20% of the biomass. Reference to percentages of a certain saturation type, such as “at least 50% monounsaturated in an 18:1 glycerolipid form” means the aliphatic side chains of the glycerolipids are at least 50% 18:1, but does not necessarily mean that at least 50% of the triglycerides are triolein (three 18:1 chains attached to a single glycerol backbone); such a profile can include glycerolipids with a mixture of 18:1 and other side chains, provided at least 50% of the total side chains are 18:1.

“Growth” means an increase in cell size, total cellular contents, and/or cell mass or weight of an individual cell, including increases in cell weight due to conversion of a fixed carbon source into intracellular oil.

“Heterotrophic cultivation” and variants thereof such as “heterotrophic culture” and “heterotrophic fermentation” refer to the intentional fostering of growth (increases in cell size, cellular contents, and/or cellular activity) in the presence of a fixed carbon source. Heterotrophic cultivation is performed in the absence of light. Cultivation in the absence of light means cultivation of microbial cells in the complete absence or near complete absence of light where the cells do not derive a meaningful amount of their energy from light (ie: greater than 0.1%).

“Heterotrophic propagation” and variants thereof refer to the intentional fostering of propagation (increases in cell numbers via mitosis) in the presence of a fixed carbon source. Heterotrophic propagation is performed in the absence of light. Propagation in the absence of light means propagation of microbial cells in the complete absence or near complete absence of light where the cells do not derive a meaningful amount of their energy from light (ie: greater than 0.1%).

“Homogenate” is biomass that has been physically disrupted.

“Hydrocarbon” is (a) a molecule containing only hydrogen and carbon atoms wherein the carbon atoms are covalently linked to form a linear, branched, cyclic, or partially cyclic backbone to which the hydrogen atoms are attached. The molecular structure of hydrocarbon compounds varies from the simplest, in the form of methane (CH₄), which is a constituent of natural gas, to the very heavy and very complex, such as some molecules such as asphaltenes found in crude oil, petroleum, and bitumens. Hydrocarbons may be in gaseous, liquid, or solid form, or any combination of these forms, and may have one or more double or triple bonds between adjacent carbon atoms in the backbone. Accordingly, the term includes linear, branched, cyclic, or partially cyclic alkanes, alkenes, lipids, and paraffin. Examples include propane, butane, pentane, hexane, octane, and squalene.

“Hydrogen:carbon ratio” is the ratio of hydrogen atoms to carbon atoms in a molecule on an atom-to-atom basis. The ratio may be used to refer to the number of carbon and hydrogen atoms in a hydrocarbon molecule. For example, the hydrocarbon with the highest ratio is methane CH₄ (4:1).

“Hydrophobic fraction” is the portion, or fraction, of a material that is more soluble in a hydrophobic phase in comparison to an aqueous phase. A hydrophobic fraction is substantially insoluble in water and usually non-polar.

“Increased lipid yield” refers to an increase in the productivity of a microbial culture by, for example, increasing dry weight of cells per liter of culture, increasing the percentage of cells that constitute lipid, or increasing the overall amount of lipid per liter of culture volume per unit time.

“In situ” means “in place” or “in its original position”. For example, a culture may contain a first yeast cell type secreting a catalyst and a second microorganism cell type secreting a substrate, wherein the first and second cell types produce the components necessary for a particular chemical reaction to occur in situ in the co-culture without requiring further separation or processing of the materials.

“Limiting concentration of a nutrient” is a concentration of a compound in a culture that limits the propagation of a cultured organism. A “non-limiting concentration of a nutrient” is a concentration that supports maximal propagation during a given culture period. Thus, the number of cells produced during a given culture period is lower in the presence of a limiting concentration of a nutrient than when the nutrient is non-limiting. A nutrient is said to be “in excess” in a culture, when the nutrient is present at a concentration greater than that which supports maximal propagation.

“Lipase” is a water-soluble enzyme that catalyzes the hydrolysis of ester bonds in water-insoluble, lipid substrates. Lipases catalyze the hydrolysis of lipids into glycerols and fatty acids.

“Lipids” are a class of molecules that are soluble in nonpolar solvents (such as ether and chloroform) and are relatively or completely insoluble in water. Lipid molecules have these properties, because they consist largely of long hydrocarbon chains which are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); nonglycerides (sphingolipids, sterol lipids including cholesterol and steroid hormones, prenol lipids including terpenoids, fatty alcohols, waxes, and polyketides); and complex lipid derivatives (sugar-linked lipids, or glycolipids, and protein-linked lipids). “Fats” are a subgroup of lipids called “triacylglycerides.”

“Lysate” is a solution containing the contents of lysed cells.

“Lysis” is the breakage of the plasma membrane and optionally the cell wall of a biological organism sufficient to release at least some intracellular content, often by mechanical, viral or osmotic mechanisms that compromise its integrity.

“Lysing” is disrupting the cellular membrane and optionally the cell wall of a biological organism or cell sufficient to release at least some intracellular content.

“Microorganism” and “microbe” are microscopic unicellular organisms.

“Naturally co-expressed” with reference to two proteins or genes means that the proteins or their genes are co-expressed naturally in a tissue or organism from which they are derived, e.g., because the genes encoding the two proteins are under the control of a common regulatory sequence or because they are expressed in response to the same stimulus.

“Oil” means any triacylglyceride (or triglyceride oil), produced by organisms, including oleaginous yeast, plants, and/or animals. “Oil,” as distinguished from “fat”, refers, unless otherwise indicated, to lipids that are generally liquid at ordinary room temperatures and pressures. For example, “oil” includes vegetable or seed oils derived from plants, including without limitation, an oil derived from soy, rapeseed, canola, palm, palm kernel, coconut, corn, olive, sunflower, cotton seed, cuphea, peanut, camelina sativa, mustard seed, cashew nut, oats, lupine, kenaf, calendula, hemp, coffee, linseed, hazelnut, euphorbia, pumpkin seed, coriander, camellia, sesame, safflower, rice, tung oil tree, cocoa, copra, pium poppy, castor beans, pecan, jojoba, jatropha, macadamia, Brazil nuts, and avocado, as well as combinations thereof.

“Oleaginous yeast” means yeast that can naturally accumulate more than 20% of their dry cell weight as lipid and are of the Dikarya subkingdom of fungi. Oleaginous yeast includes organisms such as Yarrowia lipolytica, Rhodotorula glutinis, Cryptococcus curvatus and Lipomyces starkeyi.

“Osmotic shock” is the rupture of cells in a solution following a sudden reduction in osmotic pressure. Osmotic shock is sometimes induced to release cellular components of such cells into a solution.

“Polysaccharide-degrading enzyme” is any enzyme capable of catalyzing the hydrolysis, or saccharification, of any polysaccharide. For example, cellulases catalyze the hydrolysis of cellulose.

“Polysaccharides” or “glycans” are carbohydrates made up of monosaccharides joined together by glycosidic linkages. Cellulose is a polysaccharide that makes up certain plant cell walls. Cellulose can be depolymerized by enzymes to yield monosaccharides such as xylose and glucose, as well as larger disaccharides and oligosaccharides.

“Port” means an opening in a bioreactor that allows influx or efflux of materials such as gases, liquids, and cells; a port is usually connected to tubing.

“Predominantly encapsulated” means that more than 50% and typically more than 75% to 90% of a referenced component, e.g., algal oil, is sequestered in a referenced container, which can include, e.g., a oleaginous yeast cell.

“Predominantly intact cells” and “predominantly intact biomass” mean a population of cells that comprise more than 50, and often more than 75, 90, and 98% intact cells. “Intact”, in this context, means that the physical continuity of the cellular membrane and/or cell wall enclosing the intracellular components of the cell has not been disrupted in any manner that would release the intracellular components of the cell to an extent that exceeds the permeability of the cellular membrane in culture.

“Predominantly lysed” means a population of cells in which more than 50%, and typically more than 75 to 90%, of the cells have been disrupted such that the intracellular components of the cell are no longer completely enclosed within the cell membrane.

“Proliferation” means a combination of both growth and propagation.

“Propagation” means an increase in cell number via mitosis or other cell division.

“Renewable diesel” is a mixture of alkanes (such as C10:0, C12:0, C14:0, C16:0 and C18:0) produced through hydrogenation and deoxygenation of lipids.

“Saccharification” is a process of converting biomass, usually cellulosic or lignocellulosic biomass, into monomeric sugars, such as glucose and xylose. “Saccharified” or “depolymerized” cellulosic material or biomass refers to cellulosic material or biomass that has been converted into monomeric sugars through saccharification.

“Sonication” is a process of disrupting biological materials, such as a cell, by use of sound wave energy.

“Species of furfural” is 2-furancarboxaldehyde or a derivative that retains the same basic structural characteristics.

“Stover” is the dried stalks and leaves of a crop remaining after a grain has been harvested.

“Suitable for human consumption” means a composition can be consumed by humans as dietary intake without ill health effects and can provide significant caloric intake due to uptake of digested material in the gastrointestinal tract.

“V/V” or “v/v”, in reference to proportions by volume, means the ratio of the volume of one substance in a composition to the volume of the composition. For example, reference to a composition that comprises 5% v/v yeast oil means that 5% of the composition's volume is composed of oil (e.g., such a composition having a volume of 100 mm³ would contain 5 mm³ of oil), and the remainder of the volume of the composition (e.g., 95 mm³ in the example) is composed of other ingredients.

“W/W” or “w/w”, in reference to proportions by weight, means the ratio of the weight of one substance in a composition to the weight of the composition. For example, reference to a composition that comprises 5% w/w oleaginous yeast biomass means that 5% of the composition's weight is composed of oleaginous yeast biomass (e.g., such a composition having a weight of 100 mg would contain 5 mg of oleaginous yeast biomass) and the remainder of the weight of the composition (e.g., 95 mg in the example) is composed of other ingredients.

### II. OLEAGINOUS YEAST STRAINS AND CULTURE CONDITIONS

The present invention provides methods for producing and extracting oil from oleaginous yeast biomass. The invention arose in part from the discoveries that yeast biomass can be prepared with a high oil content, the oil can be readily extracted therefrom, and that the oil can be converted into a variety of useful fuels. Yeast oil, which can comprise a mixture of saturated and mid-length fatty acids (e.g., C16 fatty acids), provides excellent starting material for the preparation of a variety of fuels and chemicals.

This section describes the types of oleaginous yeast suitable for use in the methods of the invention, the culture conditions for generating yeast biomass, the concentration steps used to prepare the biomass for further processing, and the chemical composition of the biomass prepared in accordance with the methods of the invention.

A variety of species of yeast that produce suitable oils and/or lipids can be used in accordance with the methods of the present invention, although yeast that naturally produce high levels of suitable oils and/or lipids are preferred. Considerations affecting the selection of yeast for use in the invention include, in addition to production of suitable oils or lipids for production of food products: (1) high lipid content as a percentage of cell weight; (2) ease of growth; (3) ease of propagation; (4) ease of biomass processing; and (5) glycerolipid profile.

In particular embodiments, the oleaginous yeast comprise cells that are at least 20% or more triglyceride oil by dry weight. In other embodiments, the oleaginous yeast comprises at least 25-35% or more triglyceride oil by dry weight. Generally, in these embodiments, the more oil contained in the oleaginous yeast, the more oil that can be extracted from the biomass, so oleaginous yeast that can be cultured to comprise at least 40%, at least 50%, or at least 60% or more triglyceride oil by dry weight are especially preferred. Not all types of lipids are desirable for use in fuels, as they may have an undesirable chain length or be associated with undesirable contaminants, and these considerations also influence the selection of oleaginous yeast for use in the methods of the invention.

Suitable species of oleaginous yeast for use in the invention include, but are not limited to Candida apicola, Candida sp., Cryptococcus curvatus, Cryptococcus terricolus, Debaromyces hansenii, Endomycopsis vernalis, Geotrichum carabidarum, Geotrichum cucujoidarum, Geotrichum histeridarum, Geotrichum silvicola, Geotrichum vulgare, Hyphopichia burtonii, Lipomyces lipofer, Lypomyces orentalis, Lipomyces starkeyi, Lipomyces tetrasporous, Pichia mexicana, Rodosporidium sphaerocarpum, Rhodosporidium toruloides Rhodotorula aurantiaca, Rhodotorula dairenensis, Rhodotorula diffluens, Rhodotorula glutinus, Rhodotorula glutinis var. glutinis, Rhodotorula gracilis, Rhodotorula graminis Rhodotorula minuta, Rhodotorula mucilaginosa, Rhodotorula mucilaginosa var.mucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri var. loubieri, Trichosporon montevideense, Trichosporon pullulans, Trichosporon sp., Wickerhamomyces Canadensis, Yarrowia lipolytica, and Zygoascus meyerae.

Species of oleaginous yeast for use in the invention can be identified by comparison of certain target regions of their genome with those same regions of species identified herein; preferred species are those that exhibit identity or at least a very high level of homology with the species identified herein and produce similar amounts, and similar types of, lipid as the strains specifically described herein. For examples, identification of a specific oleaginous yeast species or strain can be achieved through amplification and sequencing of genomic DNA using primers and methodology using appropriate regions of the genome, for example using the methods described in Kurtzman and Robnett, Antonie van Leeuwenhoek 73(4): 331-371 (1998), Identification and phylogeny of ascomycetous yeasts from analysis of nuclear large subunit (26S) ribosomal DNA partial sequences. Well established methods of phylogenetic analysis, such as amplification and sequencing of nuclear 18S and 26S and internal transcribed spacer (ITS) regions of ribosomal RNA genes and other conserved regions can be used by those skilled in the art to identify species of oleaginous yeasts suitable for use in the methods disclosed herein.

Thus, genomic DNA comparison can be used to identify suitable species of oleaginous yeast to be used in the present invention. Regions of conserved genomic DNA, such as, but not limited to conserved genomic sequences between 3′ regions of fungal 18S and 5′ regions of fungal 26S rRNA genes can be amplified from yeast species that may be, for example, taxonomically related to the preferred oleaginous yeasts used in the present invention and compared to the corresponding regions of those preferred species. Species that exhibit a high level of similarity are then sleeted for use in the methods of the invention. Example 4 describes genomic sequencing of conserved 3′ regions of fungal 18S and 5′ regions of fungal 26S rRNA for 48 strains of oleaginous yeast strains.

For sequence comparison to determine percent nucleotide or amino acid identity, typically one sequence acts as a reference sequence, to which test sequences are compared. In applying a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). Another example algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (at the web address www.ncbi.nlm.nih.gov).

The methods and compositions of the invention are useful for generating raw materials for food from a large class or eukaryotic, oleaginous yeast. Exemplary species cultivated and described herein include numerous species from the Dikarya subkingdom of fungi such as Rhodosporidium toruloides (Eukaryota; Fungi/Metazoa group; Fungi; Dikarya; Basidiomycota; Pucciniomycotina; Microbotryomycetes; Sporidiobolales; Rhodosporidium); Rhodotorula glutinis (Eukaryota; Fungi/Metazoa group; Fungi; Dikarya; Basidiomycota; Pucciniomycotina; Microbotryomycetes; Sporidiobolales; mitosporic Sporidiobolales; Rhodotorula); Lipomyces tetrasporus (Eukaryota; Fungi/Metazoa group; Fungi; Dikarya; Ascomycota; Saccharomyceta; Saccharomycotina; Saccharomycetes; Saccharomycetales; Lipomycetaceae; Lipomyces); Cryptococcus curvatus (Eukaryota; Fungi/Metazoa group; Fungi; Dikarya; Basidiomycota; Agaricomycotina; Tremellomycetes; Tremellales; mitosporic Tremellales; Cryptococcus); Trichosporon domesticum (Eukaryota; Fungi/Metazoa group; Fungi; Dikarya; Basidiomycota; Agaricomycotina; Tremellomycetes; Tremellales; mitosporic Tremellales; Trichosporon); Yarrowia lipolytica (Eukaryota; Fungi/Metazoa group; Fungi; Dikarya; Ascomycota; Saccharomyceta; Saccharomycotina; Saccharomycetes; Saccharomycetales; Dipodascaceae; Yarrowia); Sporobolomyces alborubescens (Eukaryota; Fungi/Metazoa group; Fungi; Dikarya; Basidiomycota; Pucciniomycotina; Microbotryomycetes; Sporidiobolales; mitosporic Sporidiobolales; Sporobolomyces); Geotrichum vulgare (Eukaryota; Fungi/Metazoa group; Fungi; Dikarya; Ascomycota; Saccharomyceta; Saccharomycotina; Saccharomycetes; Saccharomycetales; Dipodascaceae; mitosporic Dipodascaceae; Geotrichum): and Torulaspora delbrueckii (Eukaryota; Fungi/Metazoa group; Fungi; Dikarya; Ascomycota; Saccharomyceta; Saccharomycotina; Saccharomycetes; Saccharomycetales; Saccharomycetaceae; Torulaspora). Within Dikarya, the invention includes use of organisms from all sub-domains of Dikarya (Ascomycota and Basidiomycota) and taxonomic sub-classifications within Ascomycota and Basidiomycota.

Oleaginous yeast are cultured in liquid media to propagate biomass in accordance with the methods of the invention. In the methods of the invention, oleaginous yeast species are grown in a medium containing a fixed carbon source and/or fixed nitrogen source in the absence of light (heterotrophic growth). Heterotrophic growth of oleaginous yeast usually occurs in an aerobic environment. For example, heterotrophic growth for extended periods of time such as 10 to 15 or more days under limited nitrogen conditions can result in accumulation of light lipid/oil content in cells.

Oleaginous yeast culture media typically contains components such as a fixed carbon source (discussed below), a fixed nitrogen source (such as protein, soybean meal, yeast extract, cornsteep liquor, ammonia (pure or in salt form), nitrate, or nitrate salt), trace elements, optionally a buffer for pH maintenance, and phosphate (a source of phosphorous; other phosphate salts can be used).

In a particular example, a medium suitable for culturing oleaginous yeast strains is YPD medium. This medium is suitable for axenic cultures, and a 1 L volume of the medium (pH˜6.8) can be prepared by addition of 10g bacto-yeast, 20g bacto-peptone and 40g glucose into distilled water. For 1.5% agar medium, 15 g of agar can be added to 1 L of the solution. The solution is covered and autoclaved, and then stored at a refrigerated temperature prior to use. Other methods for the growth and propagation of oleaginous yeast strains to generate high lipid levels as a percentage of dry weight have been described (see for example Li et al., Enzyme and Microbial Technology (2007) 41:312-317 (demonstrating the culturing Rhodosporidium toruloides to 67.5% w/w lipid using fed batch fermentation)). High lipid/oil content in oleaginous yeast can typically be generated by increasing the length of fermentation while providing an excess of carbon source under nitrogen limitation.

Solid and liquid growth media are generally available from a wide variety of sources, and instructions for the preparation of particular media that is suitable for a wide variety of strains of oleaginous yeast can be found, for example, online at http://www.dsmz.de/microorganisms/medium/pdf/DSMZ_Medium186.pdf

Other suitable media for use with the methods of the invention can be readily identified by consulting the URL identified above, or by consulting other organizations that maintain cultures of oleaginous yeast such as Fungal Culture Collections of The World Austrian Center of Biological Resources and Applied Mycology (http://www.biotec.boku.ac.at/acbr.html); The Biomedical Fungi and Yeasts Collection (http://bccm.belspo.be/about/ihem.php); Czech Collection of Microorganisms (http://sci.muni.cz/ccm/index.html); Institut Pasteur (http://www.pasteur.fr/ip/easysite/go/03b-000011-08h/); German Collection of Microorganisms and Cell Cultures (http://www.dsmz.de/); Mychoteca Univesitatis Taurinenesis (http://web086.unito.it/cgi-bin/bioveg/documenti.pl/Show?_id=b522); Riken Bioresource Center Japan Collection of Microorganisms (http://wwwj cm.rikenjp/JCM/announce.shtml); The National Collection of Yeast Cultures (http://www.ncyc.co.uk/); ATCC (http://www.atcc.org/); Phaff Yeast Culture Collection (http://www.phaffcollection.org/).

Oleaginous yeast useful in accordance with the methods of the present invention are found in various locations and environments throughout the world. As a consequence of their isolation from other species and their resulting evolutionary divergence, the particular growth medium for optimal growth and generation of oil and/or lipid and/or protein from any particular species of microbe can be difficult or impossible to predict, but those of skill in the art can readily find appropriate media by routine testing in view of the disclosure herein. In some cases, certain strains of microorganisms may be unable to grow on a particular growth medium because of the presence of some inhibitory component or the absence of some essential nutritional requirement required by the particular strain of microorganism. The examples below provide exemplary methods of culturing various species of oleaginous yeast to accumulate high levels of lipid as a percentage of dry cell weight.

The fixed carbon source is a key component of the medium. Suitable fixed carbon sources for purposes of the present invention, include, for example, glucose, fructose, sucrose, lactose, galactose, xylose, mannose, rhamnose, arabinose, N-acetylglucosamine, glycerol, floridoside, glucuronic acid, and/or acetate. Other carbon sources for culturing oleaginous yeast in accordance with the present invention include mixtures, such as mixtures of any of the foregoing carbon sources, mixtures of glycerol and glucose, mixtures of glucose and xylose, mixtures of fructose and glucose, and mixtures of sucrose and depolymerized sugar beet pulp. Other carbon sources suitable for use in culturing oleaginous yeast include, black liquor, corn starch, depolymerized cellulosic material (derived from, for example, corn stover, sugar beet pulp, and switchgrass, for example), milk whey, molasses, potato, rice, sorghum, sucrose, sugar beet, sugar cane, and wheat. The one or more carbon source(s) can be supplied at a concentration of at least about 50 μM, at least about 100 μM, at least about 500 μM, at least about 5 mM, at least about 50 mM, and at least about 500 mM. Carbon sources of particular interest for purposes of the present invention include cellulose (in a depolymerized form), glycerol, sucrose, and sorghum, each of which is discussed in more detail below.

Thus, in various embodiments, the fixed carbon energy source used in the growth medium comprises glycerol and/or 5- and/or 6-carbon sugars, such as glucose, fructose, and/or xylose, which can be derived from sucrose and/or cellulosic material, including depolymerized cellulosic material. Multiple species of oleaginous yeast and multiple strains within a species can be grown in the presence of sucrose, depolymerized cellulosic material, and glycerol, as described in US Patent Application Publication Nos. 20090035842, 20090011480, 20090148918, respectively, and see also, PCT Patent Application Publication No. 2008/151149, each of which is incorporated herein by reference.

Thus, in one embodiment of the present invention, oleaginous yeast are cultured using depolymerized cellulosic biomass as a feedstock. As opposed to other feedstocks, such as corn starch or sucrose from sugar cane or sugar beets, cellulosic biomass (depolymerized or otherwise) is not suitable for human consumption and could potentially be available at low cost, which makes it especially advantageous for purposes of the present invention. Cellulosic biomass (e.g., stover, such as corn stover) is inexpensive and readily available; however, prior to the present invention, attempts to use this material as a feedstock for yeast have failed. In particular, such feedstocks have been found to be inhibitory to yeast growth, and yeast cannot use the 5-carbon sugars produced from cellulosic materials (e.g., xylose from hemi-cellulose).

Oleaginous yeasts can proliferate on depolymerized cellulosic material. Cellulosic materials generally include cellulose at 40-60% dry weight; hemicellulose at 20-40% dry weight; and lignin at 10-30% dry weight. Suitable cellulosic materials include residues from herbaceous and woody energy crops, as well as agricultural crops, i.e., the plant parts, primarily stalks and leaves, not removed from the fields with the primary food or fiber product. Examples include agricultural wastes such as sugarcane bagasse, rice hulls, corn fiber (including stalks, leaves, husks, and cobs), wheat straw, rice straw, sugar beet pulp, citrus pulp, citrus peels; forestry wastes such as hardwood and softwood thinnings, and hardwood and softwood residues from timber operations; wood wastes such as saw mill wastes (wood chips, sawdust) and pulp mill waste; urban wastes such as paper fractions of municipal solid waste, urban wood waste and urban green waste such as municipal grass clippings; and wood construction waste. Additional cellulosics include dedicated cellulosic crops such as switchgrass, hybrid poplar wood, and miscanthus, fiber cane, and fiber sorghum. Five-carbon sugars that are produced from such materials include xylose. Example 3 describes oleaginous yeast successfully utilizing saccharifiedcellulosic-derived sugars from a variety of sources.

Some microbes are able to process cellulosic material and directly utilize cellulosic materials as a carbon source. However, cellulosic material typically needs to be treated to increase the accessible surface area or for the cellulose to be first broken down as a preparation for microbial utilization as a carbon source. Ways of preparing or pretreating cellulosic material for enzyme digestion are well known in the art. The methods are divided into two main categories: (1) breaking apart the cellulosic material into smaller particles in order to increase the accessible surface area; and (2) chemically treating the cellulosic material to create a useable substrate for enzyme digestion.

Methods for increasing the accessible surface area include steam explosion, which involves the use of steam at high temperatures to break apart cellulosic materials. Because of the high temperature requirement of this process, some of the sugars in the cellulosic material may be lost, thus reducing the available carbon source for enzyme digestion (see for example, Chahal, D. S. et al., Proceedings of the 2^{nd} World Congress of Chemical Engineering; (1981) and Kaar et al., Biomass and Bioenergy (1998) 14(3): 277-87). Ammonia explosion allows for explosion of cellulosic material at a lower temperature, but is more costly to perform, and the ammonia might interfere with subsequent enzyme digestion processes (see for example, Dale, B. E. et al., Biotechnology and Bioengineering (1982); 12: 31-43). Another explosion technique involves the use of supercritical carbon dioxide explosion in order to break the cellulosic material into smaller fragments (see for example, Zheng et al., Biotechnology Letters (1995); 17(8): 845-850).

Methods for chemically treating the cellulosic material to create useable substrates for enzyme digestion are also known in the art. U.S. Pat. No. 7,413,882 describes the use of genetically engineered microbes that secrete beta-glucosidase into the fermentation broth and treating cellulosic material with the fermentation broth to enhance the hydrolysis of cellulosic material into glucose. Cellulosic material can also be treated with strong acids and bases to aid subsequent enzyme digestion. U.S. Pat. No. 3,617,431 describes the use of alkaline digestion to break down cellulosic materials.

Some species of oleaginous yeast can proliferate on media containing combinations of xylose and glucose, such as depolymerized cellulosic material. Thus, certain oleaginous yeasts can both utilize an otherwise inedible feedstock, such as cellulosic material (or a pretreated cellulosic material) or glycerol, as a carbon source and produce edible oils. In some cases, lignocellulosic degradation products can have an inhibitory effect on oleaginous yeast growth. One study reported that acetic acid, formic acid, furfural and vanillin (common lignocellulosic degradation products) were strong inhibitors of growth for some species of oleaginous yeasts. (Chen et al., Appl. Biochem. Biotech. (2009) 159: 591-604). In certain embodiments, minimizing lignocellulosic degradation products may be advantageous for the growth of certain oleaginous yeast species/strains.

Thus, in various embodiments of the invention, cellulosic materials are treated to increase the efficiency with which the oleaginous yeast can utilize the sugar(s) contained within the materials. The invention provides novel methods for the treatment of cellulosic materials after acid explosion so that the materials are suitable for use in a heterotrophic culture of oleaginous yeast. As discussed above, lignocellulosic biomass is comprised of various fractions, including cellulose, a crystalline polymer of beta 1,4 linked glucose (a six-carbon sugar), hemicellulose, a more loosely associated polymer predominantly comprised of xylose (a five-carbon sugar) and to a lesser extent mannose, galactose, arabinose, lignin, a complex aromatic polymer comprised of sinapyl alcohol and its derivatives, and pectins, which are linear chains of an alpha 1,4 linked polygalacturonic acid. Because of the polymeric structure of cellulose and hemicellulose, the sugars (e.g., monomeric glucose and xylose) in them are not in a form that can be efficiently used (metabolized) by many oleaginous yeast. For such microbes, further processing of the cellulosic biomass to generate the monomeric sugars that make up the polymers can be very helpful to ensuring that the cellulosic materials are efficiently utilized as a feedstock (carbon source).

Celluose or cellulosic biomass is subjected to a process, termed “explosion”, in which the biomass is treated with dilute sulfuric (or other) acid at elevated temperature and pressure. This process conditions the biomass such that it can be efficiently subjected to enzymatic hydrolysis of the cellulosic and hemicellulosic fractions into glucose and xylose monomers. The resulting monomeric sugars are termed cellulosic sugars. Cellulosic sugars can subsequently be utilized by microorganisms to produce a variety of metabolites (e.g., lipid). The acid explosion step results in a partial hydrolysis of the hemicellulose fraction to constitutent monosaccharides. These sugars can be completely liberated from the biomass with further treatment. In some embodiments, the further treatment is a hydrothermal treatment that includes washing the exploded material with hot water, which removes contaminants such as salts. This step is not necessary for cellulosic ethanol fermentations due to the more dilute sugar concentrations used in such processes. In other embodiments, the further treatment is additional acid treatment. In still other embodiments, the further treatment is enzymatic hydrolysis of the exploded material. These treatments can also be used in any combination. The type of treatment can affect the type of sugars liberated (e.g., five carbon sugars versus six carbon sugars) and the stage at which they are liberated in the process. As a consequence, different streams of sugars, whether they are predominantly five-carbon or six-carbon, can be created. These enriched five-carbon or six-carbon streams can thus be directed to specific microorganisms with different carbon utilization cabilities.

The methods of the present invention typically involve fermentation to higher cell densities than what is achieved in ethanol fermentation. Because of the higher densities of the cultures for heterotrophic cellulosic oil production, the fixed carbon source (e.g., the cellulosic derived sugar stream(s)) is preferably in a concentrated form. The glucose level of the depolymerized cellulosic material is preferably at least 300g/liter, at least 400g/liter, at least 500g/liter or at least 600g/liter prior to the cultivation step, which is optionally a fed batch cultivation in which the material is fed to the cells over time as the cells grow and accumulate lipid. Cellulosic sugar streams are not used at or near this concentration range in the production of cellulosic ethanol. Thus, in order to generate and sustain the very high cell densities during the production of lignocellulosic oil, the carbon feedstock(s) must be delivered into the heterotrophic cultures in a highly concentrated form. However, any component in the feedstream that is not a substrate for, and is not metabolized by, the oleaginous microorganism will accumulate in the bioreactor, which can lead to problems if the component is toxic or inhibitory to production of the desired end product. While ligin and lignin-derived by-products, carbohydrate-derived byproducts such as furfurals and hydroxymethyl furfurals and salts derived from the generation of the cellulosic materials (both in the explosion process and the subsequent neutralization process), and even non-metabolized pentose/hexose sugars can present problems in ethanolic fermentations, these effects are amplified significantly in a process in which their concentration in the initial feedstock is high. To achieve sugar concentrations in the 300 g/L range (or higher) for six-carbon sugars that may be used in large scale production of lignocellulosic oil described in the present invention, the concentration of these toxic materials can be 20 times higher than the concentrations typically present in ethanolic fermentations of cellulosic biomass.

The explosion process treatment of the cellulosic material utilizes significant amounts of sulfuric acid, heat and pressure, thereby liberating by-products of carbohydrates, namely furfurals and hydroxymethyl furfurals. Furfurals and hydroxymethyl furfurals are produced during hydrolysis of hemicellulose through dehydration of xylose into furfural and water. In some embodiments of the present invention, these by-products (e.g., furfurals and hydroxymethyl furfurals) are removed from the saccharified lignocellulosic material prior to introduction into the bioreactor. In certain embodiments of the present invention, the process for removal of the by-products of carbohydrates is hydrothermal treatment of the exploded cellulosic materials. In addition, the present invention provides methods in which strains capable of tolerating compounds such as furfurals or hydroxymethyl furfurals are used for lignocellulosic oil production. In another embodiment, the present invention also provides methods and microorganisms that are not only capable of tolerating furfurals in the fermentation media, but are actually able to metabolize these by-products during the production of lignocellulosic oil.

The explosion process also generates significant levels of salts. For example, typical conditions for explosion can result in conductivities in excess of 5 mS/cm when the exploded cellulosic biomass is resuspended at a ratio of 10:1 water:solids (dry weight). In certain embodiments of the present invention, the diluted exploded biomass is subjected to enzymatic saccharification, and the resulting supernatant is concentrated up to 25 fold for use in the bioreactor. The salt level (as measured by conductivity) in the concentrated sugar stream(s) can be unacceptably high (up to 1.5 M Na⁺ equivalents). Additional salts are generated upon neutralization of the exploded materials for the subsequent enzymatic saccharification process as well. The present invention provides methods for removing these salts so that the resulting concentrated cellulosic sugar stream(s) can be used in heterotrophic processes for producing lignocellulosic oil. In some embodiments, the method of removing these salts is deionization with resins, such as, but not limited to, DOWEX Marathon MR3. In certain embodiments, the deionization with resin step occurs before sugar concentration or pH adjustment and hydrothermal treatment of biomass prior to saccharification, or any combination of the preceding; in other embodiments, the step is conducted after one or more of these processes. In other embodiments, the explosion process itself is changed so as to avoid the generation of salts at unacceptably high levels. For example, a suitable alternative to sulfuric acid (or other acid) explosion of the cellulosic biomass is mechanical pulping to render the cellulosic biomass receptive to enzymatic hydrolysis (saccharification). In still other embodiments, native strains of microorganisms resistant to high levels of salts or genetically engineered strains with resistance to high levels of salts are used.

A preferred embodiment for the process of preparing of exploded cellulosic biomass for use in heterotrophic lignocellulosic oil production using oleaginous microbes involves several steps. Step I comprises adjusting the pH of the resuspended exploded cellulosic biomass to the range of 5.0-5.3 followed by washing the cellulosic biomass three times. This washing step can be accomplished by a variety of means including the use of desalting and ion exchange resins, reverse osmosis, hydrothermal treatment (as described above), or just repeated re-suspension and centrifugation in deionized water. This wash step results in a cellulosic stream whose conductivity is between 100-300 μS/cm and the removal of significant amounts of furfurals and hydroxymethyl furfurals. Decants from this wash step can be saved to concentrate five-carbon sugars liberated from the hemicellulose fraction. Step II comprises enzymatic saccharification of the washed cellulosic biomass. In a preferred embodiment, Accellerase (Genencor) is used. Step III comprises the recovery of sugars via centrifugation or decanting and rinsing of the saccharified biomass. The resulting biomass (solids) is an energy dense, lignin rich component that can be used as fuel or sent to waste. The recovered sugar stream in the centrifugation/decanting and rinse process is collected. Step IV comprises microfiltration to remove contaminating solids with recovery of the permeate. Step V comprises a concentration step which can be accomplished using a vacuum evaporator. This step can optionally include the addition of antifoam agents such as P′2000 (Sigma/Fluka), which is sometimes necessary due to the protein content of the resulting sugar feedstock.

In another embodiment of the methods of the invention, the carbon source is sucrose, including a complex feedstock comprising sucrose, such as thick cane juice from sugar cane processing. Complex feedstocks comprising sucrose include waste molasses from sugar cane processing; the use of this low-value waste product of sugar cane processing can provide significant cost savings in the production of hydrocarbons and other oils. Another complex feedstock comprising sucrose that is useful in the methods of the invention is sorghum, including sorghum syrup and pure sorghum. Sorghum syrup is produced from the juice of sweet sorghum cane. Its sugar profile consists of mainly glucose (dextrose), fructose and sucrose.

The use of cellulosic materials, glycerol, and certain other of the materials described above, as a carbon source allows conversion of inedible materials, or materials that are normally not part of the human food chain (as compared to corn glucose and sucrose from sugar cane and sugar beet), into oil suitable for use in the production of fuel. Thus, the invention provides methods for turning inedible feedstock into fuels, offering significant advantages over prior art methods that rely on the use of edible feedstocks to produce fuel, as the instant methods do not require diversion of material from the human food supply for the production of fuel.

High lipid biomass from oleaginous yeast is an advantageous material for production of fuels compared to low lipid biomass, because less yeast biomass is required to produce the same amount of oil. The lipid-rich biomass provided by the methods of the invention typically has at least 20% lipid by dry cell weight.

Process conditions can be adjusted to increase the percentage weight of cells that is lipid. For example, in certain embodiments, oleaginous yeast is cultured in the presence of a limiting concentration of one or more nutrients, such as, for example, nitrogen, phosphate, and certain metallic ions, while providing an excess of a fixed carbon source, such as glucose. Nitrogen limitation tends to increase microbial lipid yield over microbial lipid yield in a culture in which nitrogen is provided in excess. In particular embodiments, the increase in lipid yield is at least about 10%, 50%, 100%, 200%, or 500%. The microbe can be cultured in the presence of a limiting amount of a nutrient for a portion of the total culture period or for the entire period. In some embodiments, the nutrient concentration is cycled between a limiting concentration and a non-limiting concentration at least twice during the total culture period.

In a steady growth state, the cells accumulate oil (lipid) but do not undergo cell division. In one embodiment of the invention, the growth state is maintained by continuing to provide all components of the original growth media to the cells with the exception of a fixed nitrogen source. Cultivating oleaginous yeast by feeding all nutrients originally provided to the cells except a fixed nitrogen source, such as through feeding the cells for an extended period of time, results in a higher percentage of lipid by dry cell weight.

In other embodiments, high lipid biomass is generated by feeding a fixed carbon source to the cells after all fixed nitrogen has been consumed for extended periods of time, such as at least one or two weeks. In some embodiments, cells are allowed to accumulate oil in the presence of a fixed carbon source and in the absence of a fixed nitrogen source for over 10, over 15, or over 20 days. Oleaginous yeast grown using conditions described herein or otherwise known in the art can comprise at least about 20% lipid by dry weight, and often comprise 35%, 45%, 55%, 65%, and even 75% or more lipid by dry weight. Percentage of dry cell weight as lipid in microbial lipid production can therefore be improved by holding cells in a growth state in which they consume carbon and accumulate oil but do not undergo cell division.

Conditions in which nitrogen is in excess tends to increase microbial protein yield over microbial protein yield in a culture in which nitrogen is not provided in excess. For maximal protein production, the microbe is preferably cultured in the presence of excess nitrogen for the total culture period. Suitable nitrogen sources for oleaginous yeast may come from organic nitrogen sources and/or inorganic nitrogen sources.

Non-limiting examples of organic nitrogen sources are yeast extract, peptone, corn steep liquor, and corn steep powder. Non-limiting examples of preferred inorganic nitrogen sources include, for example, and without limitation, (NH₄)₂SO₄ and NH₄OH. In one embodiment, the culture media for carrying out the invention comprises only inorganic nitrogen sources. In another embodiment, the culture media for carrying out the invention comprises only organic nitrogen sources. In yet another embodiment, the culture media for carrying out the invention comprises a mixture of organic and inorganic nitrogen sources.

An example of a medium formulation used to grow oleaginous yeast include: 7 g/L KH₂PO₄; 2 g/L Na₂HPO₄; 1.5 g/L MgSO₄.7H₂O; 1.5 g/L yeast extract; 0.2 g/L CaCl₂.6H₂O; 0.1 g/L FeCl₃.6H₂O; 0.001 g/L biotin and 0.001 g/L ZnSO₄.7H₂O with a pH level adjusted to 5.5 with HCL and with 12 g/L glucose and 30 g/L NH₄Cl as a nitrogen source. Another medium that is used to grow oleaginous yeast include: 20 g/L glucose; 0.5 g/L yeast extract; 5 g/L (NH₄)₂SO₄; and 1 g/L KH₂PO₄; 0.5 g/L MgSO₄.7H₂O. One medium formulation for the growth of oleaginous in a fermentor consists of: 30 g/L glucose; 20 g/L xylose; 2 g/L (NH₄)₂SO₄; 1 g/L KH₂PO₄; and 0.5 g/L MgSO₄.7H₂O.

In the methods of the invention, a bioreactor or fermentor is used to culture oleaginous yeast cells through the various phases of their physiological cycle. As an example, an inoculum of lipid-producing oleaginous yeast cells is introduced into the medium; there is a lag period (lag phase) before the cells begin to propagate. Following the lag period, the propagation rate increases steadily and enters the log, or exponential, phase. The exponential phase is in turn followed by a slowing of propagation due to decreases in nutrients such as nitrogen, increases in toxic substances, and quorum sensing mechanisms. After this slowing, propagation stops, and the cells enter a stationary phase or steady growth state, depending on the particular environment provided to the cells. For obtaining lipid rich biomass, the culture is typically harvested well after then end of the exponential phase, which may be terminated early by allowing nitrogen or another key nutrient (other than carbon) to become depleted, forcing the cells to convert the carbon sources, present in excess, to lipid. Culture condition parameters can be manipulated to optimize total oil production, the combination of lipid species produced, and/or production of a specific oil.

To produce biomass for use in food, oleaginous yeast are preferably fermented in large quantities in liquid, such as in suspension cultures as an example. Bioreactors such as steel fermentors (5000 liter, 10,000 liter, 40,000 liter, and larger volumes are used in various embodiments of the invention) can accommodate very large culture volumes. Bioreactors also typically allow for the control of culture conditions such as temperature, pH, oxygen tension, and carbon dioxide levels. For example, bioreactors are typically configurable, for example, using ports attached to tubing, to allow gaseous components, like oxygen or nitrogen, to be bubbled through a liquid culture.

Bioreactors can be configured to flow culture media though the bioreactor throughout the time period during which the oleaginous yeast reproduce and increase in number. In some embodiments, for example, media can be infused into the bioreactor after inoculation but before the cells reach a desired density. In other instances, a bioreactor is filled with culture media at the beginning of a culture, and no more culture media is infused after the culture is inoculated. In other words, the oleaginous yeast biomass is cultured in an aqueous medium for a period of time during which the yeast reproduce and increase in number; however, quantities of aqueous culture medium are not flowed through the bioreactor throughout the time period. Thus in some embodiments, aqueous culture medium is not flowed through the bioreactor after inoculation.

Bioreactors equipped with devices such as spinning blades and impellers, rocking mechanisms, stir bars, means for pressurized gas infusion can be used to subject oleaginous yeast cultures to mixing. Mixing may be continuous or intermittent. As briefly mentioned above, bioreactors are often equipped with various ports that, for example, allow the gas content of the culture to be manipulated. To illustrate, part of the volume of a bioreactor can be gas rather than liquid, and the gas inlets of the bioreactor to allow pumping of gases into the bioreactor. Gases that can be beneficially pumped into a bioreactor include air, air/CO₂ mixtures, noble gases, such as argon, and other gases. Bioreactors are typically equipped to enable the user to control the rate of entry of a gas into the bioreactor. As noted above, increasing gas flow into a bioreactor can be used to increase mixing of the culture.

Increased gas flow affects the turbidity of the culture as well. Turbulence can be achieved by placing a gas entry port below the level of the aqueous culture media so that gas entering the bioreactor bubbles to the surface of the culture. One or more gas exit ports allow gas to escape, thereby preventing pressure buildup in the bioreactor. Preferably a gas exit port leads to a “one-way” valve that prevents contaminating microorganisms from entering the bioreactor.

The specific examples of bioreactors, culture conditions, and heterotrophic growth and propagation methods described herein can be combined in any suitable manner to improve efficiencies of microbial growth and lipid and/or protein production.

Oleaginous yeast cultures generated according to the methods described above yield oleaginous yeast biomass in fermentation media. To prepare the biomass for extraction of oil, the biomass is typically concentrated, or harvested, from the fermentation medium. At the point of harvesting the oleaginous yeast biomass from the fermentation medium, the biomass comprises predominantly intact cells suspended in an aqueous culture medium. To concentrate the biomass, a dewatering step is performed. Dewatering or concentrating refers to the separation of the biomass from fermentation broth or other liquid medium and so is solid-liquid separation. Thus, during dewatering, the culture medium is removed from the biomass (for example, by draining the fermentation broth through a filter that retains the biomass), or the biomass is otherwise removed from the culture medium. Common processes for dewatering include centrifugation, filtration, and the use of mechanical pressure. These processes can be used individually or in any combination.

Centrifugation involves the use of centrifugal force to separate mixtures. During centrifugation, the more dense components of the mixture migrate away from the axis of the centrifuge, while the less dense components of the mixture migrate towards the axis. By increasing the effective gravitational force (i.e., by increasing the centrifugation speed), more dense material, such as solids, separate from the less dense material, such as liquids, and so separate out according to density. Centrifugation of biomass and broth or other aqueous solution forms a concentrated paste comprising the oleaginous yeast cells. Centrifugation does not remove significant amounts of intracellular water. In fact, after centrifugation, there may still be a substantial amount of surface or free moisture in the biomass (e.g., upwards of 70%), so centrifugation is not considered to be a drying step.

Filtration can also be used for dewatering. One example of filtration that is suitable for the present invention is tangential flow filtration (TFF), also known as cross-flow filtration. Tangential flow filtration is a separation technique that uses membrane systems and flow force to separate solids from liquids. For an illustrative suitable filtration method, see Geresh, Carb. Polym. 50; 183-189 (2002), which describes the use of a MaxCell AIG Technologies 0.45 uM hollow fiber filter. Also see, for example, Millipore Pellicon® devices, used with 100 kD, 300 kD), 1000 kD (catalog number P2C01MC01), 0.1 uM (catalog number P2VVPPV01), 0.22 uM (catalog number P2GVPPV01), and 0.45 uM membranes (catalog number P2HVMPV01). The retentate preferably does not pass through the filter at a significant level, and the product in the retentate preferably does not adhere to the filter material. TFF can also be performed using hollow fiber filtration systems. Filters with a pore size of at least about 0.1 micrometer, for example about 0.12, 0.14, 0.16, 0.18, 0.2, 0.22, 0.45, or at least about 0.65 micrometers, are suitable. Preferred pore sizes of TFF allow solutes and debris in the fermentation broth to flow through, but not microbial cells.

Dewatering can also be effected with mechanical pressure directly applied to the biomass to separate the liquid fermentation broth from the microbial biomass sufficient to dewater the biomass but not to cause predominant lysis of cells. Mechanical pressure to dewater microbial biomass can be applied using, for example, a belt filter press. A belt filter press is a dewatering device that applies mechanical pressure to a slurry (e.g., microbial biomass taken directly from the fermentor or bioreactor) that is passed between the two tensioned belts through a serpentine of decreasing diameter rolls. The belt filter press can actually be divided into three zones: the gravity zone, where free draining water/liquid is drained by gravity through a porous belt; a wedge zone, where the solids are prepared for pressure application; and a pressure zone, where adjustable pressure is applied to the gravity drained solids.

After concentration, oleaginous yeast biomass is processed, as described hereinbelow, to prepare it for oil extraction.

Oleaginous yeast biomass with a high percentage of oil/lipid accumulation by dry weight has been generated using different methods of culture, including methods known in the art. Oleaginous yeasts with a higher percentage of accumulated oil/lipid are useful in accordance with the present invention. Candida 107 was shown to be able to accumulate up to 40% lipid wt/wt under nitrogen limiting conditions (Gill et al., Appl and Environ Microbiology (1977) pp. 231-239). Li et al. demonstrated the production of Rhodosporidium toruloids 44 in fed-batch cultures to a lipid conent of 48% w/w (Li et al., Enzyme and Microbial Technology (2007) 41:312-317. Yarrowia lipolytica has been show to be able to produce between 0.44-0.54 g of lipid per gram of biomass when using animal fat (stearin) as a carbon source (Panpanikolaou et al., Appl Microbiol Biotechnol (2002) 58:308-312.

Biomass generated by the culture methods described herein and useful in accordance with the present invention comprises at least 10% oil by dry weight. In some embodiments, the biomass comprises at least 25%, at least 50%, at least 55%, or at least 60% oil by dry weight. In some embodiments, the biomass comprises from 10-90% oil, from 25-75% oil, from 40-75% oil, or from 50-70% oil by dry weight.

The oil of the biomass described herein, or extracted from the biomass for use in the methods and compositions of the present invention can comprise glycerolipids with one or more distinct fatty acid ester side chains. Glycerolipids are comprised of a glycerol molecule esterified to one, two or three fatty acid molecules, which can be of varying lengths and have varying degrees of saturation. The oil composition, i.e., the properties and proportions of the fatty acid constituents of the glycerolipids, can be manipulated by combining biomass or oil from at least two distinct species of oleaginous yeast (or a strain of oleaginous yeast and another oil producing microbe). In some embodiments, at least two of the distinct species of microbe have different glycerolipid profiles. The distinct species of microbe can be cultured together or separately as described herein, preferably under heterotrophic conditions, to generate the respective oils. Different species of microbe can contain different percentages of distinct fatty acid constituents in the cell's glycerolipids.

Yarrowia lipolytica has been genetically engineered. An object of the invention is using engineered strains of Yarrowia lipolytica containing lipid modification enzymes to make fuel oil rather than highly unsaturated nutritional oils such as EPA. Examples of engineering Yarrowia using lipid modification enzymes, along with genetic engineering methodology and vectors are described in U.S. Pat. Nos. 7,465,565 and 7,273,746 and U.S. patent application Ser. Nos. 10/840,579, 11/613,420, 11/714,377 and 11/264,737.

### III. EXTRACTION OF TRIGLYCERIDE OIL FROM OLEAGINOUS YEAST

For the production of fuel in accordance with the methods of the invention lipids produced by oleaginous yeast cells of the invention are harvested, or otherwise collected or separated, from the fermentation broth by any convenient means. Lipids (oil) is then isolated (extracted from the cells) by any convenient means, such as, for example whole cell solvent extraction or pressing. During this isolation process, the cells are disrupted or lysed, so that intracellular and cell membrane/cell wall-associated lipids as well as extracellular hydrocarbons can be separated from the remaining cell mass. Once extracted, the oil (lipid) is further refined to produce fuels, hydrocarbon compositions, and/or oleochemicals. Each of these steps is described in more detail below.

After completion of culturing, the oleaginous yeast are separated from the fermentation broth. Optionally, the separation is effected by centrifugation to generate a concentrated paste. Centrifugation does not remove significant amounts of intracellular water from the oleaginous yeast and so is considered a dewatering, not a drying, step. The biomass can then optionally be washed with a washing solution (e.g., DI water) to remove fermentation broth and debris. As described in further detail below, the dewatered microbial biomass may also be dried (oven dried, lyophilized, and the like) and conditioned prior to cell disruption (lysis). In some embodiments, however, cells are lysed without separation from some or all of the fermentation broth after the fermentation is complete; for example, the cells can be at a ratio to fermentation broth of less than 1:1 v:v cells to fermentation broth (extracellular liquid) when the cells are lysed.

The step of lysing a oleaginous yeast (also referred to as cell lysis) can be achieved by any convenient means, including heat-induced lysis, adding a base, adding an acid, using enzymes such as proteases and polysaccharide degradation enzymes such as amylases, using ultrasound, mechanical lysis (i.e., subjecting the biomass to pressure sufficient to lyse the cells, termed “pressing”), using osmotic shock, infection with a lytic virus, and/or expression of one or more lytic genes. Lysis is performed to release intracellular molecules which have been produced by the oleaginous yeast. Each of these methods for lysing oleaginous yeast can be used as a single method or in combination simultaneously or sequentially. The extent of cell disruption can be observed by microscopic analysis. Using one or more of the methods described herein, typically more than 70% cell breakage is observed. Preferably, cell breakage (lysis) is more than 80% complete, more preferably more than 90% complete, and most preferably about 100% complete.

In one embodiment of the present invention, the step of lysing oleaginous yeast comprises heating of a cellular suspension containing the oleaginous yeast. In this embodiment, the fermentation broth containing the oleaginous yeast (or a suspension of oleaginous yeast isolated from the fermentation broth) is heated until the oleaginous yeast, i.e., the cell walls and membranes of oleaginous yeast degrade or breakdown. Typically, temperatures applied are at least 50° C. Higher temperatures, such as at least 60° C., at least 70° C., at least 80° C., at least 90° C., at least 100° C., at least 110° C., at least 120° C., at least 130° C. or higher are used for more efficient cell lysis. Lysing cells by heat treatment can be performed by boiling the oleaginous yeast. Alternatively, heat treatment (without boiling) can be performed in an autoclave. The heat treated lysate may be cooled for further treatment. Cell disruption can also be performed by steam treatment, i.e., through addition of pressurized steam. Steam treatment of microbes for cell disruption is described, for example, in U.S. Pat. No. 6,750,048. In some embodiments, steam treatment may be achieved by sparging steam into the fermentor and maintaining the broth at a desired temperature for less than about 90 minutes, preferably less than about 60 minutes, and more preferably less than about 30 minutes.

In another embodiment of the present invention, the step of lysing a oleaginous yeast comprises adding a base to a cellular suspension containing the oleaginous yeast. The base should be strong enough to hydrolyze at least a portion of the proteinaceous compounds of the oleaginous yeasts used. Bases which are useful for solubilizing proteins are known in the art of chemistry. Exemplary bases which are useful in the methods of the present invention include, but are not limited to, hydroxides, carbonates and bicarbonates of lithium, sodium, potassium, calcium, and mixtures thereof. A preferred base is KOH. Base treatment of microalgae for cell disruption is described, for example, in U.S. Pat. No. 6,750,048.

In another embodiment of the present invention, the step of lysing a oleaginous yeast comprises adding an acid to a cellular suspension containing the oleaginous yeast. Acid lysis can be effected using an acid at a concentration of 10-500 mN or preferably 40-160 nM. Acid lysis is preferably performed at above room temperature (e.g., at 40-160°, and preferably a temperature of 50-130°. For moderate temperatures (e.g., room temperature to 100° C. and particularly room temperature to 65°, acid treatment can usefully be combined with sonication or other cell disruption methods.

In another embodiment of the present invention, the step of lysing a microorganism comprises lysing the microorganism by using an enzyme. Preferred enzymes for lysing a microorganism are proteases and polysaccharide-degrading enzymes such as hemicellulase (e.g., hemicellulase from Aspergillus niger; Sigma Aldrich, St. Louis, Mo.; #H2125), pectinase (e.g., pectinase from Rhizopus sp.; Sigma Aldrich, St. Louis, Mo.; #P2401), Mannaway 4.0 L (Novozymes), cellulase (e.g., cellulase from Trichoderma viride or Chlorella or a Chlorella virus; Sigma Aldrich, St. Louis, Mo.; #C9422;), driselase (e.g., driselase from Basidiomycetes sp.; Sigma Aldrich, St. Louis, Mo.; #D9515; Streptomyces griseus protease, chymotrypsin, proteinase K, proteases listed in Degradation of Polylactide by Commercial Proteases, Oda Y et al., Journal of Polymers and the Environment, Volume 8, Number 1, January 2000, pp. 29-32(4), Alcalase 2.4 FG (Novozymes), and Flavourzyme 100 L (Novozymes). Any combination of a protease and a polysaccharide-degrading enzyme can also be used, including any combination of the preceding proteases and polysaccharide-degrading enzymes.

In another embodiment, lysis can be performed using an expeller press. In this process, biomass is forced through a screw-type device at high pressure, lysing the cells and causing the intracellular lipid to be released and separated from the protein and fiber (and other components) in the cell. This process is described in more detail below.

In another embodiment of the present invention, the step of lysing oleaginous yeast is performed by using ultrasound, i.e., sonication. Thus, cells can also by lysed with high frequency sound. The sound can be produced electronically and transported through a metallic tip to an appropriately concentrated cellular suspension. This sonication (or ultrasonication) disrupts cellular integrity based on the creation of cavities in cell suspension.

In another embodiment of the present invention, the step of lysing oleaginous yeast is performed by mechanical lysis. Cells can be lysed mechanically and optionally homogenized to facilitate hydrocarbon (e.g., lipid) collection. For example, a pressure disrupter can be used to pump a cell containing slurry through a restricted orifice valve. High pressure (up to 1500 bar) is applied, followed by an instant expansion through an exiting nozzle. Cell disruption is accomplished by three different mechanisms: impingement on the valve, high liquid shear in the orifice, and sudden pressure drop upon discharge, causing an explosion of the cell. The method releases intracellular molecules. Alternatively, a ball mill can be used. In a ball mill, cells are agitated in suspension with small abrasive particles, such as beads. Cells break because of shear forces, grinding between beads, and collisions with beads. The beads disrupt the cells to release cellular contents. Cells can also be disrupted by shear forces, such as with the use of blending (such as with a high speed or Waring blender as examples), the french press, or even centrifugation in case of weak cell walls, to disrupt cells.

In another embodiment of the present invention, the step of lysing oleaginous yeast is performed by applying an osmotic shock.

In another embodiment of the present invention, the step of lysing oleaginous yeast comprises infection of the oleaginous yeast with a lytic virus. A wide variety of viruses are known to lyse oleaginous yeast and are suitable for use in the present invention, and the selection and use of a particular lytic virus for a particular oleaginous yeast is within the level of skill in the art.

In another embodiment of the present invention, the step of lysing oleaginous yeast comprises autolysis. In this embodiment, oleaginous yeast according to the invention is genetically engineered to produce a lytic protein that will lyse oleaginous yeast. This lytic gene can be expressed using an inducible promoter so that the cells can first be grown to a desirable density in a fermentor, followed by induction of the promoter to express the lytic gene to lyse the cells. In one embodiment, the lytic gene encodes a polysaccharide-degrading enzyme. In certain other embodiments, the lytic gene is a gene from a lytic virus. Expression of lytic genes is preferably done using an inducible promoter, such as a promoter active in oleaginous yeast that is induced by a stimulus such as the presence of a small molecule, light, heat, and other stimuli.

Once the cells are lysed, various methods are available for separating lipids from cellular lysates produced by the above methods. For example, lipids and lipid derivatives such as fatty aldehydes, fatty alcohols, and hydrocarbons such as alkanes can be extracted with a hydrophobic solvent such as hexane (see Frenz et al. 1989, Enzyme Microb. Technol., 11:717). Lipids and lipid derivatives can also be extracted using liquefaction (see for example Sawayama et al. 1999, Biomass and Bioenergy 17:33-39 and Inoue et al. 1993, Biomass Bioenergy 6(4):269-274); oil liquefaction (see for example Minowa et al. 1995, Fuel 74(12):1735-1738); and supercritical CO₂ extraction (see for example Mendes et al. 2003, Inorganica Chimica Acta 356:328-334). Miao and Wu describe a protocol of the recovery of microalgal lipid from a culture of Chlorella prototheocoides in which the cells were harvested by centrifugation, washed with distilled water and dried by freeze drying. The resulting cell powder was pulverized in a mortar and then extracted with n-hexane. Miao and Wu, Biosource Technology (2006) 97:841-846.

Thus, lipids, lipid derivatives and hydrocarbons generated by oleaginous yeast of the present invention can be recovered by extraction with an organic solvent. In some cases, the preferred organic solvent is hexane. Typically, the organic solvent is added directly to the lysate without prior separation of the lysate components. In one embodiment, the lysate generated by one or more of the methods described above is contacted with an organic solvent for a period of time sufficient to allow the lipid and/or hydrocarbon components to form a solution with the organic solvent. In some cases, the solution can then be further refined to recover specific desired lipid or hydrocarbon components. Hexane extraction methods are well known in the art.

Another method for removing the oil from the biomass involves lysing the cells by subjecting them to pressure under conditions such that the oil is separated from the biomass. This process is termed “pressing” and can conveniently be practiced using a device known as an expeller press. To prepare the biomass for pressing, the biomass is first dewatered using one or more of the above dewatering techniques. The moisture content of the microbial biomass at the time of pressing (“conditioned feedstock” is used to describe the biomass at the time of pressing) can dramatically affects the yield of oil obtained in the pressing step, and the optimal moisture level is generally below 6%. For example, optimal moisture content can be no more than 5%, no more than 1%, or no more than 0.5% by weight. The optimal moisture level can depend on several factors, including but not limited to the percent lipids (oil) as measured by dry cell weight (DCW) or the amount of fiber in the biomass. In some embodiments of the methods of the invention, such as, for example, those in which a bulking agent is employed, dewatering alone provides a suitable moisture content of the oleaginous yeast biomass that is then conditioned for the pressing step. In other methods and embodiments of the invention, dewatered biomass is subjected to a drying step and then conditioned prior to the pressing step (in which oil is extracted from the biomass).

Drying, as referred to herein, refers to the removal of some or all of the free moisture or surface moisture of the oleaginous yeast biomass. Like dewatering, the drying process should not result in significant loss of oil from the yeast biomass. Thus, the drying step should typically not cause lysis of a significant number of the microbial cells, because in most cases, the lipids are located in intracellular compartments of the microbial biomass. Several methods of drying microbial biomass known in the art for other purposes are suitable for use in the methods of the present invention. Microbial biomass after the free moisture or surface moisture has been removed is referred to as dried microbial biomass. If no further moisture removal occurs in the conditioning or moisture reduction occurs via the addition of a dry bulking agent prior to the pressing step, then the dried microbial biomass should contain less than 6% moisture by weight.

In various embodiments, the dry microbial biomass has a moisture content in the range of 0.1% to 5% by weight. In various embodiments, the dry microbial biomass has a moisture content of less than 4% by weight. In various embodiments, the dry microbial biomass has a moisture content in the range of 0.5% to 3.5% by weight. In various embodiments, the dry microbial biomass has a moisture content in the range of 0.1% to 3% by weight. Non-limiting examples of drying methods suitable for use in preparing dry microbial biomass in accordance with the methods of the invention include lyophilization and the use of dryers such as a drum dryer, spray dryer, and a tray dryer, each of which is described below.

Lyophilization, also known as freeze drying or cryodessication, is a dehydration process that is typically used to preserve a perishable material. The lyophilization process involves the freezing of the material and then reducing the surrounding pressure and adding enough heat to allow the frozen water in the material to sublime from the solid phase to gas. In the case of lyophilizing oleaginous yeast biomass, the cell wall can act as a cryoprotectant that prevents degradation of the intracellular lipids during the freeze dry process.

Drum dryers are one of the most economical methods for drying large amounts of microbial biomass. Drum dryers, or roller dryers, consist of two large steel cylinders that turn toward each other and are heated from the inside by steam. In some embodiments, the oleaginous yeast biomass is applied to the outside of the large cylinders in thin sheets. Through the heat from the steam, the oleaginous yeast biomass is then dried, typically in less than one revolution of the large cylinders, and the resulting dry oleaginous yeast biomass is scraped off of the cylinders by a steel blade. The resulting dry oleaginous yeast biomass has a flaky consistency. In various embodiments, the oleaginous yeast biomass is first dewatered and then dried using a drum dryer. More detailed description of a drum dryer can be found in U.S. Pat. No. 5,729,910, which discloses a rotary drying drum.

Spray drying is a commonly used method of drying a liquid feed using a hot gas. A spray dryer takes a liquid stream (e.g., containing the oleaginous yeast biomass) and separates the solute as a solid and the liquid into a vapor. The liquid input stream is sprayed through a nozzle into a hot vapor stream and vaporized. Solids form as moisture quickly leaves the droplets. The nozzle of the spray dryer is adjustable, and typically is adjusted to make the droplets as small as possible to maximize heat transfer and the rate of water vaporization. The resulting dry solids may have a fine, powdery consistency, depending on the size of the nozzle used. In other embodiments, spray dryers can use a lyophilization process instead of steam heating to dry the material.

Tray dryers are typically used for laboratory work and small pilot scale drying operations. Tray dryers work on the basis of convection heating and evaporation. Fermentation broth containing the oleaginous yeast biomass can be dried effectively from a wide range of cell concentrations using heat and an air vent to remove evaporated water.

Flash dryers are typically used for drying solids that have been de-watered or inherently have a low moisture content. Also known as “pneumatic dryers”, these dryers typically disperse wet material into a stream of heated air (or gas) which conveys it through a drying duct. The heat from the airstream (or gas stream) dries the material as it is conveyed through the drying duct. The dried product is then separated using cyclones and/or bag filters. Elevated drying temperatures can be used with many products, because the flashing off of surface moisture instantly cools the drying gas/air without appreciably increasing the product temperature. More detailed descriptions of flash dryers and pneumatic dryers can be found in U.S. Pat. No. 4,214,375, which describes a flash dryer, and U.S. Pat. Nos. 3,789,513 and 4,101,264, which describe pneumatic dryers.

Regardless of the method selected for a drying step, the objective of the drying step is to reduce moisture content in the oleaginous yeast biomass. If moisture is not removed from the dry oleaginous yeast biomass during the conditioning step or reduced via the addition of a dry bulking agent, then the moisture content should be less than 6% by weight. Typically, the dry oleaginous yeast biomass (conditioned feedstock) suitable for pressing has a moisture content of about 0.1% to 6% by weight, including in various embodiments, a moisture content of at least 0.5% by weight, at least 3% by weight, and at least 3.5% by weight. Moisture may be added back to the biomass, if necessary, after drying to adjust moisture content to the optimal level. If the dry oleaginous yeast biomass will be admixed with a dry bulking agent (see subsection D) or conditioned in a manner that will reduce moisture content further (see subsection C), then higher (above 6% by weight) moisture content may be acceptable, as bulking agents and/or conditioning can, in some embodiments, reduce the moisture content to the desired optimal level.

Dewatered and/or dried oleaginous yeast biomass is conditioned prior to the pressing step. Conditioning of the oleaginous yeast biomass helps to achieve desired levels of oil extraction. Conditioning refers to heating the biomass to a temperature in the range of 70° C. to 150° C. (160° F. to 300° F.) and changing the physical or physiochemical nature of the oleaginous yeast biomass to improve oil yields in the subsequent oil extraction (pressing) step. Conditioning oleaginous yeast biomass results in the production of “conditioned feedstock.” In addition to heating or “cooking” the biomass, non-limiting examples of conditioning the biomass include adjusting the moisture content within the dry oleaginous yeast biomass, subjecting the dry oleaginous yeast biomass to a low pressure “pre-press”, subjecting the dry oleaginous yeast biomass to cycles of heating and cooling, subjecting the dry oleaginous yeast biomass to an expander, and/or adjusting the particle size of the dry oleaginous yeast biomass.

The conditioning step can include techniques (e.g., heating or application or pressure) that overlap in part with techniques used in the drying or pressing steps. However, the primary goals of these steps are different: the primary goal of the drying step is the removal of some or all of the free moisture or surface moisture from the oleaginous yeast biomass. The primary goal of the conditioning step is to heat the biomass, which can optionally result in the removal of intracellular water from, i.e., adjusting the intracellular moisture content of, the oleaginous yeast biomass and/or altering the physical or physiochemical nature of the oleaginous yeast biomass without substantial release of lipids to facilitate release of oil during the pressing step. The primary the goal of the pressing step is to release oil from the oleaginous yeast biomass or conditioned feedstock, i.e., the extraction of the oil.

In various embodiments, conditioning involves altering, or adjusting, the moisture content of the oleaginous yeast biomass by the application of heat, i.e., heat conditioning. Heat conditioning, as used herein, refers to heat treatment (either direct or indirect) of oleaginous yeast biomass. The moisture content of the oleaginous yeast biomass can be adjusted by conditioning using heat (either direct or indirect), which is typically done, if at all, after a drying step. Even though the biomass may be dried by any of the above described methods, the moisture content of the oleaginous yeast biomass after drying can range, for example, from 3% to 15% moisture by weight, i.e., 5-10% moisture by weight. Such a moisture range may not be optimal for maximal oil recovery in the pressing step. Therefore, there may be benefit in heat-conditioning dewatered and/or dry oleaginous yeast biomass to adjust the moisture level to a level (below 6%) optimal for maximal oil recovery.

Heat conditioners used in oil seed processing are suitable for use in conditioning oleaginous yeast biomass in accordance with the methods of the present invention. Vertical stacked conditioners have been used in the art for “cooking” oil seeds. These consist of a series of three to six closed, superimposed cylindrical steel pans. Each pan is independently jacketed for steam heating on both sides and bottom and is equipped with a sweep-type stirrer mounted close to the bottom, and operated by a common shaft extending through the entire series of pans. The temperature of the heat conditioner is also adjustable through regulation of the steam heating. There is an automatically operated gate in the bottom of each pan, except the last, for discharging the contents to the pan below. The top pan is provided with spray jets, for the addition of moisture to the seed, and each of the low pans is provided with an exhaust pipe and fan for removal of moisture. Thus, it is possible to control the moisture of the seed or, in the methods of the invention, the oleaginous yeast biomass, not only with respect to final moisture content but also at each stage of the operation. In this respect, a conditioning step of heating oleaginous yeast biomass for an extended period of time (10-60 minutes for example) provides the effect of not only reducing moisture but also altering the biophysical nature of the oleaginous yeast biomass beyond any heating effects that might occur in a subsequent pressing step, i.e., simply from friction of the material as it is forced through, e.g., a press.

For the heat conditioning of oleaginous yeast biomass, the optimal time and temperature that the biomass spends in a vertical stacked conditioner can vary depending on the moisture level of the biomass after drying. Heat conditioning (sometimes referred to as “cooking”) should not result in burning or scorching significant amounts of the oleaginous yeast biomass during cooking. Depending on the moisture content of the oleaginous yeast biomass prior to heat conditioning, i.e., for very low levels of moisture, it may be beneficial or even necessary to moisten the biomass before heat conditioning to avoid burning or scorching.

Heating the oil-bearing oleaginous yeast biomass before pressing can aid in the liberation of oil from and/or accessing the oil-laden compartments of the cells. Oil-bearing oleaginous yeast biomass contains the oil in compartments made of cellular components such as proteins and phospholipids. Repetitive cycles of heating and cooling can denature the proteins and alter the chemical structure of the cellular components of these oil compartments and thereby provide better access to the oil during the subsequent extraction process. Thus, in various embodiments of the invention, the oleaginous yeast biomass is conditioned to prepare conditioned feedstock that is used in the pressing step, and the conditioning step involves heating and, optionally, one or more cycles of heating and cooling.

If no further heat conditioning or other conditioning that alters moisture content is to be performed, and if no bulking agent that will alter moisture content is to be added, then the conditioned feedstock resulting from heat conditioning should contain less than 6% moisture by weight. In various embodiments, the conditioned feedstock has a moisture content in the range of 0.1% to 5% by weight. In various embodiments, the conditioned feedstock has a moisture content of less than 4% by weight. In various embodiments, the conditioned feedstock has a moisture content in the range of 0.5% to 3.5% by weight. In various embodiments, the conditioned feedstock has a moisture content in the range of 0.1% to 3% by weight.

In addition to heating the biomass, conditioning can, in some embodiments, involve the application of pressure to the oleaginous yeast biomass. To distinguish this type of conditioning from the pressure applied during oil extraction (the pressing step), this type of conditioning is referred to as a “pre-press.” The pre-press is conducted at low pressure, a pressure lower than that used for oil extraction in the pressing step. Ordinary high-pressure expeller (screw) presses may be operated at low pressure for this pre-press conditioning step. Pre-pressing the biomass at low pressure may aid in breaking open the cells to allow for better flow of oil during the subsequent high pressure pressing; however, pre-pressing does not cause a significant amount (e.g. more than 5%) of the oil to separate from the oleaginous yeast biomass. Also, the friction and heat generated during the pre-press may also help break open the oil compartments in the cells. Pre-pressing the biomass at low pressure also changes the texture and particle size of the biomass, because the biomass will extrude out of the press in a pellet-like form. In some embodiments, an extruder (see discussion below) is used to achieve the same or similar results as a low pressure pre-press conditioning step. In some embodiments, the pellets of conditioned biomass are further processed to achieve an optimal particle size for the subsequent full pressure pressing.

Thus, another parameter relevant to optimal extraction of oil from oleaginous yeast biomass is the particle size. Typically, the optimum particle size for an oil expeller press (screw press) is approximately 1/16^{th} of an inch thick. Factors that may affect the range of particle size include, but are not limited to, the method used to dry the oleaginous yeast biomass and/or the addition of a bulking agent (press aid) to the biomass. If the biomass is tray dried, e.g., spread wet onto a tray and then dried in an oven, the resulting dried oleaginous yeast biomass may need to be broken up into uniform pieces of the optimal particle size to make it optimal for pressing in an expeller press. The same is true if a bulking agent is added to the oleaginous yeast biomass before the drying process. Thus, conditioning may involve a step that results in altering the particle size or average particle size of the oleaginous yeast biomass. Machines such as hammer mills or flakers may be employed in accordance with the methods of the invention to adjust the thickness and particle size of the oil-bearing oleaginous yeast biomass.

In similar fashion, improved oil extraction can result from altering other physical properties of the dried oleaginous yeast biomass. In particular, the porosity and/or the density of the oleaginous yeast biomass can affect oil extraction yields. In various embodiments of the methods of the invention, conditioning of the biomass to alter its porosity and/or density is performed. Commonly used prior to hexane or other solvent extraction of oil from oil seeds, expanders and extruders increase the porosity and the bulk density of the feedstock. In accordance with the methods of the present invention, expanders and extruders can be employed to condition the oleaginous yeast biomass before oil extraction and do not cause a significant amount (e.g. more than 5%) of oil to separate from the oleaginous yeast biomass. Both expanders and extruders are low-shear machines that heat, homogenize, and shape oil-bearing material into collets or pellets. Expanders and extruders work similarly; both have a worm/collar setup inside a shaft such that, as it moves the material inside the shaft, mechanical pressure and shearing break open the cells. The biggest difference between expanders and extruders is that the expander has a collar at the end of the shaft, which forces the material inside through the collar at high pressure. The sudden high pressure (and change in pressure) causes the moisture in the material to vaporize, thus “puffing” or expanding the material using the internal moisture. Extruders do not have this collar and, therefore, the material that is extruded is in the form of pellets. Thus, extruders and expanders can be used in accordance with the methods of the invention to condition the dry oleaginous yeast biomass. The extruder/expanders can break open the cells, freeing the intracellular lipids, and can also change the porosity and the bulk density of the material. These changes in the physical properties of the feedstock may be advantageous in subsequent oil extraction.

The above-described conditioning methods can be used alone or in combination in accordance with the methods of the invention to achieve the optimal conditioned feedstock for subsequent oil extraction. Thus, the conditioning step involves the application of heat and optionally pressure to the biomass. In various embodiments, the conditioning step comprises heating the biomass at a temperature in the range of 70° C. to 150° C. (160° F. to 300° F.). In various embodiments, the heating is performed using a vertical stacked shaker. In various embodiments, the conditioning step further comprises treating the dry biomass with an expander or extruder to shape and/or homogenize the biomass.

In various embodiments of the invention, a bulking agent or press aid is added to the oleaginous yeast biomass, which may be either dry or hydrated (i.e., biomass that has not been dried or that contains significant, i.e., more than 6% by weight, moisture, including biomass in fermentation broth that has not been subjected to any process to remove or separate water) oleaginous yeast biomass or conditioned feedstock, prior to the pressing step. In various embodiments, the bulking agent has an average particle size of less than 1.5 mm. In various embodiments, the bulking agent is selected from the group consisting of cellulose, corn stover, dried rosemary, soybean hulls, spent biomass (biomass of reduced lipid content relative to the biomass from which it was prepared), sugar cane bagasse, and switchgrass. In various embodiments, the bulking agent is spent biomass (see subsection G below) that comprises between 15% and 40% dietary fiber by weight and/or less than 10% oil by weight.

Thus, the addition of a press aid or bulking agent may be advantageous in some embodiments of the invention. A press aid is often added to oil seeds prior to screw press oil extraction. When there is high oil content and low fiber in the biomass, feeding the biomass through a press can result in an emulsion. This results in low oil yields, because the oil is trapped within the solids. One way in accordance with the methods of the invention to improve the yield in such instances is to add fiber to the biomass in the form of a bulking agent, also known as a “press aid” or “pressing aid”. Bulking agents are typically high fiber additives that work by adjusting the total fiber content of the oleaginous yeast biomass to an optimal range. Optimal fiber content for a typical oil seed may range from 10-20%. In accordance with the methods of the present invention, it may be helpful to adjust the fiber content of the oleaginous yeast biomass to the same or a similar range for optimal oil extraction. Suitable pressing aids include, but are not limited to, switchgrass, rice straw, sugar beet pulp, sugar cane bagasse, soybean hulls, dry rosemary, cellulose, corn stover and the like. In some embodiments, the spent biomass of reduced lipid content from a previous press is used as a bulking agent. In some applications, especially when the oil is going to be used in a food application or is going to be consumed, the pressing aid used in mixing with the oleaginous yeast biomass (dry or hydrated) or conditioned feedstock will be selected to meet regulatory requirements (for use as a foodstuff). Thus, bulking agents, when incorporated into a biomass, change the physiochemical properties of the biomass so as to facilitate more uniform application of pressure to cells in the biomass.

In some cases, the bulking agent can be added to the oleaginous yeast biomass after it has been dried, but not yet conditioned. In such cases, it may advantageous to mix the dry oleaginous yeast biomass with the desired amount of the press aid and then condition the oleaginous yeast biomass and the press aid together before feeding to a screw press. In other cases, the press aid can be added to a hydrated oleaginous yeast biomass before the oleaginous yeast biomass has been subjected to any separation or dewatering processes, drying, or conditioning. In such cases, the press aid can be added directly to the fermentation broth containing the oleaginous yeast biomass before any dewatering or other step.

The invention provides various methods relating to the extraction of oil from oleaginous yeast biomass that employ the bulking agents described above. In one method, hydrated oleaginous yeast biomass suitable for oil extraction is prepared by adding a bulking agent to the biomass and drying the mixture obtained thereby to a moisture content less than 6% by weight, thereby forming a dried bulking agent/biomass mixture. In another method, oil is extracted from oleaginous yeast biomass by co-drying hydrated oleaginous yeast biomass comprising at least 20% oil (including at least 40% oil) by weight and a bulking agent to form a dried bulking agent/biomass mixture; reducing the moisture content in the mixture to less than 4% by weight, i.e., by drying and/or conditioning; and pressing the reduced moisture content mixture to extract oil therefrom, thereby forming spent biomass of reduced lipid content. In another method, increased yields of oil are obtained from oleaginous yeast biomass comprising at least 20% lipid by weight by co-drying the oleaginous yeast biomass with a bulking agent, because the co-dried mixture will, upon pressing, release more oil than can be obtained from the biomass under the same conditions in the absence of a bulking agent. In various embodiments of these and other methods of the invention, the hydrated oleaginous yeast biomass is contained in fermentation broth that has not been subjected to processes to separate or remove water from the biomass.

Thus, in accordance with the methods of the invention conditioned feedstock, optionally comprising a bulking agent, is subjected to pressure in a pressing step to extract oil, producing oil separated from the spent biomass. The pressing step involves subjecting pressure sufficient to extract oil from the conditioned feedstock. Cell lysis will occur during this step, if the feedstock hasn't been subjected to conditions that lyse some of the cells prior to the pressing step. Thus, in some embodiments, the conditioned feedstock that is pressed in the pressing step comprises oil predominantly or completely encapsulated in cells of the biomass. In other embodiments, the biomass comprises predominantly lysed cells and the oil is thus primarily not encapsulated in cells.

In various embodiments of the different aspects of the invention, the pressing step will involve subjecting the conditioned feedstock to at least 20,000 psi of pressure. In various embodiments, the pressing step involves the application of pressure for a first period of time and then application of a higher pressure for a second period of time. This process may be repeated one or more times (“oscillating pressure”). In various embodiments, more than 5 cycles of oscillating pressure are applied. In various embodiments, one or more of the subsequent cycles may exert an average pressure that is higher than the average pressure exerted in one or more earlier cycles. For example and without limitation, the average pressure in the last cycle can be at least 2-fold higher than the average pressure in the first or any earlier cycle. In various embodiments, moisture content of conditioned feedstock is controlled during the pressing step. In various embodiments, the moisture is controlled in a range of from 0.1% to 3% by weight.

In various embodiments, the pressing step is conducted with an expeller press. In various embodiments, the pressing step is conducted in a continuous flow mode. In various embodiments, the oiling rate is at least 500g/min. to no more than 1000g/min. In various continuous flow embodiments, the expeller press is a device comprising a continuously rotating worm shaft within a cage having a feeder at one end and a choke at the opposite end, having openings within the cage is utilized. The conditioned feedstock enters the cage through the feeder, and rotation of the worm shaft advances the feedstock along the cage and applies pressure to the feedstock disposed between the cage and the choke, the pressure releasing oil through the openings of cage and extruding spent biomass from the choke end of the cage. In various embodiments, the cage has an internal length that is between at least ten times to at least 20 times its internal diameter. In various embodiments, the cage comprises a plurality of elongated bars with at least some of the elongated bars separated by one or more spacers, the bars resting on a frame, wherein the one or more spacers between the bars form the openings, and oil is released through the openings to a collecting vessel fluidly coupled with the cage. In various embodiments, the spacers between the elongated bars are of different thicknesses thereby allowing variation of the space between each elongated bar. In various embodiments, either the spacers or the gaps between the bars are from 0.005 to 0.020 inches thick.

In various embodiments, the pressure increases by a factor of between 10 and 17 from the feeder end to the choke end of the cage. In various embodiments, the pressure along the cage does not increase by more than 100% of the pressure at the feeder end of the cage per linear foot of the cage between the feeder and choke ends of the cage. In various embodiments, the power consumed by the device does not increase by more than 10% when fully loaded with biomass or conditioned feedstock relative to running empty. In various embodiments, the residence time of feedstock in the barrel of the device is no longer than 5-10 min. In various embodiments, either the temperature of the device or the pressure exerted by the device or both are monitored and/or controlled.

In various embodiments, pressure is controlled by adjusting rotational velocity of a worm shaft. In various embodiments, including those in which pressure is not controlled, an expeller (screw) press comprising a worm shaft and a barrel can be used. In various embodiments, the barrel has a length and a channel having a diameter sized to receive the worm shaft, and wherein the barrel length is at least 10 to 15 times greater than the channel diameter. In various embodiments, the barrel of the press has an entrance and an exit and the diameter of the worm shaft increases from the entrance to the exit, and the pressing comprises increasing the pressure from the entrance to the exit of the barrel; in various embodiments, the pressure at the exit is 12 to 16 times higher than the pressure at the entrance. In various embodiments, the expeller (screw) press comprises a worm shaft and a barrel having a first channel and a second channel, both channels concentric and sized to receive the worm shaft, wherein the first channel has a first diameter and the second channel has a second diameter different than the first diameter. In various embodiments, the conditioned feedstock remains resident in the barrel of the screw press for 5 to 10 minutes.

In various embodiments, the expeller (screw) press comprises a worm shaft disposed in a barrel lined with a plurality of elongate bars separated by one or more spacers therebetween, the spacers creating a gap between the elongate bars. In such a press, pressure can be controlled by adjusting the gap by changing the size or number of spacers between the elongate bars, and/or if the press has a space between an outer surface of the worm shaft and an inner surface of the elongate bars, pressure can be controlled by replacing at least some of the elongate bars with different sized bars so as to change the space. In various embodiments, the press comprises an output aperture and an adjustable choke coupled therewith, and pressure is controlled by adjusting the choke to increase or decrease the pressure. In various embodiments, the expeller (screw) press comprises a worm shaft disposed in a barrel, and pressure is controlled by adjusting a gap between an outer surface of the worm shaft and an inside surface of the barrel.

Expeller presses (screw presses) are routinely used for mechanical extraction of oil from soybeans and oil seeds. Generally, the main sections of an expeller press include an intake, a rotating feeder screw, a cage or barrel, a worm shaft and an oil pan. The expeller press is a continuous cage press, in which pressure is developed by a continuously rotating worm shaft. An extremely high pressure, approximately 20,000-40,000 pounds per square inch, is built up in the cage or barrel through the action of the worm working against an adjustable choke, which constricts the discharge of the pressed cake (spent biomass) from the end of the barrel. In various embodiments, a French Oil Mill, pilot plant scale screw press, Model L250 (Piqua, Ohio), is used.

Oleaginous yeast biomass or conditioned feedstock is supplied to the expeller press via an intake. A rotating feeder screw advances the material supplied from the intake into the barrel where it is then compressed by rotation of the worm shaft. Oil extracted from the material is then collected in an oil pan and then pumped to a storage tank. The remaining spent biomass is then extruded out of the press as a cake and can be collected for additional processing (see subsection G below). The cake may be pelletized.

The worm shaft is associated with a collar setup and is divided into sections. The worm and collar setup within each section is customizable. The worm shaft is responsible for conveying biomass (feedstock) through the press. It may be characterized as having a certain diameter and a thread pitch. Changing shaft diameter and pitch can increase or decrease the pressure and shear stress applied to feedstock as it passes through the press. The collar's purpose is to increase the pressure on the feedstock within the press and also apply a shear stress to the biomass.

The press load in terms of electrical current required to run the press loaded with oleaginous yeast biomass (conditioned feedstock) is usually not more than about 10% of the electrical current required to run the press empty, and this suggests that the power required to press oleaginous yeast biomass (conditioned feedstock disclosed herein is lower than other typical power requirements from the oil seed industry where the full press load is greater than 10% of the electrical current required to run the press empty of an oil seed feedstock.

The worm shaft preferably is tapered so that its outer diameter increases along the longitudinal length away from the barrel entrance. This decreases the gap between the worm shaft and the inside of the barrel thus creating greater pressure and shear stress as the biomass travels through the barrel. Additionally, the interior of the barrel is made up of flat steel bars separated by spacers (also referred to as shims), which are set edgewise around the periphery of the barrel, and are held in place by a heavy cradle-type cage. Adjusting the shim between the bars controls the gap between the bars which helps the extracted oil to drain as well as also helping to regulate barrel pressure. The shims are often from 0.003″ thick to 0.030″ thick and preferably from 0.005″ to 0.020″ thick, although other thicknesses may also be employed. Additionally, the bars may be adjusted, thereby creating sections within the barrel.

As the feed material is pressed or moved down the barrel, significant heat is generated by friction. In some cases, the amount of heat is controlled using a water jacketed cooling system that surrounds the barrel. Because of the extreme pressure, oil that is pressed from a screw press or expeller press contains a proportion of “foots” or solid material from the biomass that flows out with the oil between the bars. The foots can be screened, drained and fed back into the press along with unpressed feedstock. Temperature sensors may be disposed at various locations around the barrel to monitor and aid in temperature control. Additionally, pressure sensors may also be attached to the barrel at various locations to help monitor and control the pressure.

Various operating characteristics of the expeller (screw) press can be expressed or analyzed as a compression ratio. Compression ratio is the ratio of the volume of material displaced per revolution of the worm shaft at the beginning of the barrel divided by the volume of material displaced per revolution of the worm shaft at the end of the barrel. For example, due to increasing compression ratios the pressure may be 10 to 18 times higher at the end of the barrel as compared with the beginning of the barrel. Internal barrel length may be at least ten times or even thirteen times the internal barrel diameter. Typical compression ratio for a screw or expeller press ranges from 1 to 18, depending on the feed material.

Residence time of the feed material in an expeller (screw) press may affect the amount of oil recovery. Increased residence time in the press gives the feedstock more exposure to the shear stress and pressure generated by the press, which may yield higher oil recovery. Residence time of the feedstock depends on the speed at which the press is run and the length vs. diameter of the screw press (or L/D). The greater the ratio of the length of the shaft to the diameter of the shaft, the longer the residence time of the feedstock (when rotational speed is held at a constant).

The resulting pressed solids or cake (spent biomass of reduced oil content relative to the feedstock supplied to the screw press) is expelled from the expeller press through the discharge cone at the end of the barrel/shaft. The choke utilizes a hydraulic system to control the exit aperture on the expeller press. A fully optimized oil press operation can extract most of the available oil in the oil-bearing material. For example, optimized conditions for oil extraction from soybeans using an expeller press leaves about 5% residual oil; similar yields can be obtained from oleaginous yeast biomass (conditioned feedstock) in accordance with the methods of the invention. A variety of factors can affect the residual oil content in the pressed cake. These factors include, but are not limited to, the ability of the press to rupture oil-containing cells and cellular compartments and the composition of the oil-bearing material itself, which can have an affinity for the expelled oil. In some cases, the oil-bearing material may have a high affinity for the expelled oil and can absorb the expelled oil back into the material, thereby trapping it. In that event, the oil remaining in the spent biomass can be re-pressed or subjected to solvent extraction, as described herein, to recover the oil.

### IV. FUEL AND OLEOCHEMICAL PRODUCTION FROM EXTRACTED TRIGLYCERIDE OIL

Once the oil has been extracted from the biomass, whether by pressing or by solvent extraction or both, the oil is generally subjected to additional chemical reactions in accordance with the methods of the invention to make useful products such as fuels, hydrocarbon compositions, and/or oleochemicals. Before these various chemical reactions are described in more detail, however, it should be noted that lipids and lipid derivatives such as fatty aldehydes, fatty alcohols, and hydrocarbons such as alkanes produced by cells as described herein can be modified by the use of one or more enzymes, including a lipase, as described in more detail below, and that these modifications may take place prior to oil extraction from the biomass. For example, when the hydrocarbons are in the extracellular environment (fermentation broth) of the cells, the one or more enzymes can be added to that environment (the fermentor) under conditions in which the enzyme modifies the hydrocarbon or completes its synthesis from a hydrocarbon precursor. Alternatively, the hydrocarbons can be partially, or completely, isolated from the cellular material before addition of one or more catalysts such as enzymes. Such catalysts are exogenously added, and their activity occurs outside the cell or in vitro.

In accordance with the methods of the invention, lipids and hydrocarbons produced by cells in vivo, or enzymatically modified in vitro, can be optionally further processed by a variety of chemical means. The processing can include “cracking” to reduce the size, and thus increase the hydrogen:carbon ratio, of hydrocarbon molecules. Catalytic and thermal cracking methods are routinely used in hydrocarbon and triglyceride oil processing. Catalytic methods involve the use of a catalyst, such as a solid acid catalyst. The catalyst can be silica-alumina or a zeolite, which result in the heterolytic, or asymmetric, breakage of a carbon-carbon bond to result in a carbocation and a hydride anion. These reactive intermediates then undergo either rearrangement or hydride transfer with another hydrocarbon. The reactions can thus regenerate the intermediates to result in a self-propagating chain mechanism. Hydrocarbons can also be processed to reduce, optionally to zero, the number of carbon-carbon double, or triple, bonds therein. Hydrocarbons can also be processed to remove or eliminate a ring or cyclic structure therein. Hydrocarbons can also be processed to increase the hydrogen:carbon ratio. This can include the addition of hydrogen (“hydrogenation”) and/or the “cracking” of hydrocarbons into smaller hydrocarbons.

Thermal methods involve the use of elevated temperature and pressure to reduce hydrocarbon size. An elevated temperature of about 800° C. and pressure of about 700 kPa can be used. These conditions generate “light,” a term that is sometimes used to refer to hydrogen-rich hydrocarbon molecules (as distinguished from photon flux), while also generating, by condensation, heavier hydrocarbon molecules which are relatively depleted of hydrogen. The methodology provides homolytic, or symmetrical, breakage and produces alkenes, which may be optionally enzymatically saturated as described above.

Catalytic and thermal methods are standard in plants for hydrocarbon processing and oil refining. Thus hydrocarbons produced by cells as described herein can be collected and processed or refined via conventional means. See Hillen et al. (Biotechnology and Bioengineering, Vol. XXIV:193-205 (1982)) for a report on hydrocracking of microalgae-produced hydrocarbons. In alternative embodiments, the fraction is treated with another catalyst, such as an organic compound, heat, and/or an inorganic compound. For processing of lipids into biodiesel, a transesterification process is used as described herein.

Hydrocarbons produced via methods of the present invention are useful in a variety of industrial applications. For example, the production of linear alkylbenzene sulfonate (LAS), an anionic surfactant used in nearly all types of detergents and cleaning preparations, utilizes hydrocarbons generally comprising a chain of 10-14 carbon atoms. See, for example, U.S. Pat. Nos. 6,946,430; 5,506,201; 6,692,730; 6,268,517; 6,020,509; 6,140,302; 5,080,848; and 5,567,359. Surfactants, such as LAS, can be used in the manufacture of personal care compositions and detergents, such as those described in U.S. Pat. Nos. 5,942,479; 6,086,903; 5,833,999; 6,468,955; and 6,407,044.

Increasing interest is directed to the use of hydrocarbon components of biological origin in fuels, such as biodiesel, renewable diesel, and jet fuel, since renewable biological starting materials that may replace starting materials derived from fossil fuels are available, and the use thereof is desirable. There is an urgent need for methods for producing hydrocarbon components from biological materials. The present invention fulfills this need by providing methods for production of biodiesel, renewable diesel, and jet fuel using the lipids generated by the methods described herein as a biological material to produce biodiesel, renewable diesel, and jet fuel.

Traditional diesel fuels are petroleum distillates rich in paraffinic hydrocarbons. They have boiling ranges as broad as 370° to 780° F., which are suitable for combustion in a compression ignition engine, such as a diesel engine vehicle. The American Society of Testing and Materials (ASTM) establishes the grade of diesel according to the boiling range, along with allowable ranges of other fuel properties, such as cetane number, cloud point, flash point, viscosity, aniline point, sulfur content, water content, ash content, copper strip corrosion, and carbon residue. Technically, any hydrocarbon distillate material derived from biomass or otherwise that meets the appropriate ASTM specification can be defined as diesel fuel (ASTM D975), jet fuel (ASTM D1655), or as biodiesel if it is a fatty acid methyl ester (ASTM D6751).

After extraction, lipid and/or hydrocarbon components recovered from the oleaginous yeast biomass described herein can be subjected to chemical treatment to manufacture a fuel for use in diesel vehicles and jet engines.

Biodiesel is a liquid which varies in color—between golden and dark brown—depending on the production feedstock. It is practically immiscible with water, has a high boiling point and low vapor pressure. Biodiesel refers to a diesel-equivalent processed fuel for use in diesel-engine vehicles. Biodiesel is biodegradable and non-toxic. An additional benefit of biodiesel over conventional diesel fuel is lower engine wear. Typically, biodiesel comprises C14-C18 alkyl esters. Various processes convert biomass or a lipid produced and isolated as described herein to diesel fuels. A preferred method to produce biodiesel is by transesterification of a lipid as described herein. A preferred alkyl ester for use as biodiesel is a methyl ester or ethyl ester.

Biodiesel produced by a method described herein can be used alone or blended with conventional diesel fuel at any concentration in most modern diesel-engine vehicles. When blended with conventional diesel fuel (petroleum diesel), biodiesel may be present from about 0.1% to about 99.9%. Much of the world uses a system known as the “B” factor to state the amount of biodiesel in any fuel mix. For example, fuel containing 20% biodiesel is labeled B20. Pure biodiesel is referred to as B 100.

Biodiesel can also be used as a heating fuel in domestic and commercial boilers. Existing oil boilers may contain rubber parts and may require conversion to run on biodiesel. The conversion process is usually relatively simple, involving the exchange of rubber parts for synthetic parts due to biodiesel being a strong solvent. Due to its strong solvent power, burning biodiesel will increase the efficiency of boilers. Biodiesel can be used as an additive in formulations of diesel to increase the lubricity of pure Ultra-Low Sulfur Diesel (ULSD) fuel, which is advantageous because it has virtually no sulfur content. Biodiesel is a better solvent than petrodiesel and can be used to break down deposits of residues in the fuel lines of vehicles that have previously been run on petrodiesel.

Biodiesel can be produced by transesterification of triglycerides contained in oil-rich biomass. Thus, in another aspect of the present invention a method for producing biodiesel is provided. In a preferred embodiment, the method for producing biodiesel comprises the steps of (a) cultivating a lipid-containing oleaginous yeast using methods disclosed herein (b) lysing a lipid-containing oleaginous yeast to produce a lysate, (c) isolating lipid from the lysed oleaginous yeast, and (d) transesterifying the lipid composition, whereby biodiesel is produced. Methods for growth of a oleaginous yeast, lysing a oleaginous yeast to produce a lysate, treating the lysate in a medium comprising an organic solvent to form a heterogeneous mixture and separating the treated lysate into a lipid composition have been described above and can also be used in the method of producing biodiesel.

The lipid profile of the biodiesel is usually highly similar to the lipid profile of the feedstock oil. Other oils provided by the methods and compositions of the invention can be subjected to transesterification to yield biodiesel with lipid profiles including (a) at least 4% C8-C14; (b) at least 0.3% C8; (c) at least 2% C10; (d) at least 2% C12; and (3) at least 30% C8-C14.

Lipid compositions can be subjected to transesterification to yield long-chain fatty acid esters useful as biodiesel. Preferred transesterification reactions are outlined below and include base catalyzed transesterification and transesterification using recombinant lipases. In a base-catalyzed transesterification process, the triacylglycerides are reacted with an alcohol, such as methanol or ethanol, in the presence of an alkaline catalyst, typically potassium hydroxide. This reaction forms methyl or ethyl esters and glycerin (glycerol) as a byproduct.

Animal and plant oils are typically made of triglycerides which are esters of free fatty acids with the trihydric alcohol, glycerol. In transesterification, the glycerol in a triacylglyceride (TAG) is replaced with a short-chain alcohol such as methanol or ethanol. A typical reaction scheme is as follows:

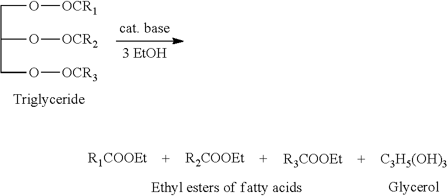

In this reaction, the alcohol is deprotonated with a base to make it a stronger nucleophile. Commonly, ethanol or methanol is used in vast excess (up to 50-fold). Normally, this reaction will proceed either exceedingly slowly or not at all. Heat, as well as an acid or base can be used to help the reaction proceed more quickly. The acid or base are not consumed by the transesterification reaction, thus they are not reactants but catalysts. Almost all biodiesel has been produced using the base-catalyzed technique as it requires only low temperatures and pressures and produces over 98% conversion yield (provided the starting oil is low in moisture and free fatty acids).

Transesterification has also been carried out, as discussed above, using an enzyme, such as a lipase instead of a base. Lipase-catalyzed transesterification can be carried out, for example, at a temperature between the room temperature and 80° C., and a mole ratio of the TAG to the lower alcohol of greater than 1:1, preferably about 3:1. Lipases suitable for use in transesterification include, but are not limited to, those listed in Table 1. Other examples of lipases useful for transesterification are found in, e.g. U.S. Pat. Nos. 4,798,793; 4,940,845 5,156,963; 5,342,768; 5,776,741 and WO89/01032. Such lipases include, but are not limited to, lipases produced by oleaginous yeasts of Rhizopus, Aspergillus, Candida, Mucor, Pseudomonas, Rhizomucor, Candida, and Humicola and pancreas lipase.

TABLE 1
Lipases suitable for use in transesterification.

Aspergillus niger lipase ABG73614, Candida antarctica lipase B
(novozym-435) CAA83122, Candida cylindracea lipase AAR24090,
Candida lipolytica lipase (Lipase L; Amano Pharmaceutical Co., Ltd.),
Candida rugosa lipase (e.g., Lipase-OF; Meito Sangyo Co., Ltd.),
Mucor miehei lipase (Lipozyme IM 20), Pseudomonas fluorescens
lipase AAA25882, Rhizopus japonicas lipase (Lilipase A-10FG)
Q7M4U7_1, Rhizomucor miehei lipase B34959, Rhizopus
oryzae lipase (Lipase F) AAF32408, Serratia marcescens
lipase (SM Enzyme) ABI13521, Thermomyces lanuginosa lipase
CAB58509, Lipase P (Nagase ChemteX Corporation), and Lipase QLM
(Meito Sangyo Co., Ltd., Nagoya, Japan)

One challenge to using a lipase for the production of fatty acid esters suitable for biodiesel is that the price of lipase is much higher than the price of sodium hydroxide (NaOH) used by the strong base process. This challenge has been addressed by using an immobilized lipase, which can be recycled. However, the activity of the immobilized lipase must be maintained after being recycled for a minimum number of cycles to allow a lipase-based process to compete with the strong base process in terms of the production cost. Immobilized lipases are subject to poisoning by the lower alcohols typically used in transesterification. U.S. Pat. No. 6,398,707 (issued Jun. 4, 2002 to Wu et al.) describes methods for enhancing the activity of immobilized lipases and regenerating immobilized lipases having reduced activity. Some suitable methods include immersing an immobilized lipase in an alcohol having a carbon atom number not less than 3 for a period of time, preferably from 0.5-48 hours, and more preferably from 0.5-1.5 hours. Some suitable methods also include washing a deactivated immobilized lipase with an alcohol having a carbon atom number not less than 3 and then immersing the deactivated immobilized lipase in a vegetable oil for 0.5-48 hours.

In particular embodiments, a recombinant lipase is expressed in the same oleaginous yeasts that produce the lipid on which the lipase acts. Suitable recombinant lipases include those listed above in Table 1 and/or having GenBank Accession numbers listed above in Table 1, or a polypeptide that has at least 70% amino acid identity with one of the lipases listed above in Table 1 and that exhibits lipase activity. In additional embodiments, the enzymatic activity is present in a sequence that has at least about 75%, at least about 80%, at least about 85%, at least about 90%, at least about 95%, or at least about 99% identity with one of the above described sequences, all of which are hereby incorporated by reference as if fully set forth. DNA encoding the lipase and selectable marker is preferably codon-optimized cDNA. Methods of recoding genes are described in U.S. Pat. No. 7,135,290.

The common international standard for biodiesel is EN 14214. ASTM D6751 is the most common biodiesel standard referenced in the United States and Canada. Germany uses DIN EN 14214 and the UK requires compliance with BS EN 14214. Basic industrial tests to determine whether the products conform to these standards typically include gas chromatography, HPLC, and others. Biodiesel meeting the quality standards is very non-toxic, with a toxicity rating (LD₅₀) of greater than 50 mL/kg.

Although biodiesel that meets the ASTM standards has to be non-toxic, there can be contaminants which tend to crystallize and/or precipitate and fall out of solution as sediment. Sediment formation is particularly a problem when biodiesel is used at lower temperatures. The sediment or precipitates may cause problems such as decreasing fuel flow, clogging fuel lines, clogging filters, etc. Processes are well-known in the art that specifically deal with the removal of these contaminants and sediments in biodiesel in order to produce a higher quality product. Examples for such processes include, but are not limited to, pretreatment of the oil to remove contaminants such as phospholipids and free fatty acids (e.g., degumming, caustic refining and silica adsorbent filtration) and cold filtration. Cold filtration is a process that was developed specifically to remove any particulates and sediments that are present in the biodiesel after production. This process cools the biodiesel and filters out any sediments or precipitates that might form when the fuel is used at a lower temperature. Such a process is well known in the art and is described in US Patent Application Publication No. 2007-0175091. Suitable methods may include cooling the biodiesel to a temperature of less than about 38° C. so that the impurities and contaminants precipitate out as particulates in the biodiesel liquid. Diatomaceous earth or other filtering material may then added to the cooled biodiesel to form a slurry, which may then filtered through a pressure leaf or other type of filter to remove the particulates. The filtered biodiesel may then be run through a polish filter to remove any remaining sediments and diatomaceous earth, so as to produce the final biodiesel product.

The Cold Soak Filterability by the ASTM D6751 A1 method of the biodiesel produced is 120 seconds for a volume of 300 ml. This test involves filtration of 300 ml of B100, chilled to 40° F. for 16 hours, allowed to warm to room temp, and filtered under vacuum using 0.7 micron glass fiber filter with stainless steel support. Oils of the invention can be transesterified to generate biodiesel with a cold soak time of less than 120 seconds, less than 100 seconds, and less than 90 seconds.

Subsequent processes may also be used if the biodiesel will be used in particularly cold temperatures. Such processes include winterization and fractionation. Both processes are designed to improve the cold flow and winter performance of the fuel by lowering the cloud point (the temperature at which the biodiesel starts to crystallize). There are several approaches to winterizing biodiesel. One approach is to blend the biodiesel with petroleum diesel. Another approach is to use additives that can lower the cloud point of biodiesel. Another approach is to remove saturated methyl esters indiscriminately by mixing in additives and allowing for the crystallization of saturates and then filtering out the crystals. Fractionation selectively separates methyl esters into individual components or fractions, allowing for the removal or inclusion of specific methyl esters. Fractionation methods include urea fractionation, solvent fractionation and thermal distillation.

Another valuable fuel provided by the methods of the present invention is renewable diesel, which comprises alkanes, such as C10:0, C12:0, C14:0, C16:0 and C18:0 and thus, are distinguishable from biodiesel. High quality renewable diesel conforms to the ASTM D975 standard. The lipids produced by the methods of the present invention can serve as feedstock to produce renewable diesel. Thus, in another aspect of the present invention, a method for producing renewable diesel is provided. Renewable diesel can be produced by at least three processes: hydrothermal processing (hydrotreating); hydroprocessing; and indirect liquefaction. These processes yield non-ester distillates. During these processes, triacylglycerides produced and isolated as described herein, are converted to alkanes.

In one embodiment, the method for producing renewable diesel comprises (a) cultivating a lipid-containing oleaginous yeast using methods disclosed herein (b) lysing the oleaginous yeast to produce a lysate, (c) isolating lipid from the lysed oleaginous yeast, and (d) deoxygenating and hydrotreating the lipid to produce an alkane, whereby renewable diesel is produced. Lipids suitable for manufacturing renewable diesel can be obtained via extraction from microbial biomass using an organic solvent such as hexane, or via other methods, such as those described in U.S. Pat. No. 5,928,696. Some suitable methods may include mechanical pressing and centrifuging.

In some methods, the oleaginous yeast lipid is first cracked in conjunction with hydrotreating to reduce carbon chain length and saturate double bonds, respectively. The material is then isomerized, also in conjunction with hydrotreating. The naptha fraction can then be removed through distillation, followed by additional distillation to vaporize and distill components desired in the diesel fuel to meet an ASTM D975 standard while leaving components that are heavier than desired for meeting the D975 standard. Hydrotreating, hydrocracking, deoxygenation and isomerization methods of chemically modifying oils, including triglyceride oils, are well known in the art. See for example European patent applications EP1741768 (A1); EP1741767 (A1); EP1682466 (A1); EP1640437 (A1); EP1681337 (A1); EP1795576 (A1); and U.S. Pat. Nos. 7,238,277; 6,630,066; 6,596,155; 6,977,322; 7,041,866; 6,217,746; 5,885,440; 6,881,873.

In one embodiment of the method for producing renewable diesel, treating the lipid to produce an alkane is performed by hydrotreating of the lipid composition. In hydrothermal processing, typically, biomass is reacted in water at an elevated temperature and pressure to form oils and residual solids. Conversion temperatures are typically 300° to 660° F., with pressure sufficient to keep the water primarily as a liquid, 100 to 170 standard atmosphere (atm). Reaction times are on the order of 15 to 30 minutes. After the reaction is completed, the organics are separated from the water. Thereby a distillate suitable for diesel is produced.

In some methods of making renewable diesel, the first step of treating a triglyceride is hydroprocessing to saturate double bonds, followed by deoxygenation at elevated temperature in the presence of hydrogen and a catalyst. In some methods, hydrogenation and deoxygenation occur in the same reaction. In other methods deoxygenation occurs before hydrogenation. Isomerization is then optionally performed, also in the presence of hydrogen and a catalyst. Naphtha components are preferably removed through distillation. For examples, see U.S. Pat. Nos. 5,475,160 (hydrogenation of triglycerides); 5,091,116 (deoxygenation, hydrogenation and gas removal); 6,391,815 (hydrogenation); and 5,888,947 (isomerization).

One suitable method for the hydrogenation of triglycerides includes preparing an aqueous solution of copper, zinc, magnesium and lanthanum salts and another solution of alkali metal or preferably, ammonium carbonate. The two solutions may be heated to a temperature of about 20° C. to about 85° C. and metered together into a precipitation container at rates such that the pH in the precipitation container is maintained between 5.5 and 7.5 in order to form a catalyst. Additional water may be used either initially in the precipitation container or added concurrently with the salt solution and precipitation solution. The resulting precipitate may then be thoroughly washed, dried, calcined at about 300° C. and activated in hydrogen at temperatures ranging from about 100° C. to about 400° C. One or more triglycerides may then be contacted and reacted with hydrogen in the presence of the above-described catalyst in a reactor. The reactor may be a trickle bed reactor, fixed bed gas-solid reactor, packed bubble column reactor, continuously stirred tank reactor, a slurry phase reactor, or any other suitable reactor type known in the art. The process may be carried out either batchwise or in continuous fashion. Reaction temperatures are typically in the range of from about 170° C. to about 250° C. while reaction pressures are typically in the range of from about 300 psig to about 2000 psig. Moreover, the molar ratio of hydrogen to triglyceride in the process of the present invention is typically in the range of from about 20:1 to about 700:1. The process is typically carried out at a weight hourly space velocity (WHSV) in the range of from about 0.1 hr⁻¹ to about 5 hr⁻¹. One skilled in the art will recognize that the time period required for reaction will vary according to the temperature used, the molar ratio of hydrogen to triglyceride, and the partial pressure of hydrogen. The products produced by such hydrogenation processes include fatty alcohols, glycerol, traces of paraffins and unreacted triglycerides. These products are typically separated by conventional means such as, for example, distillation, extraction, filtration, crystallization, and the like.

Petroleum refiners use hydroprocessing to remove impurities by treating feeds with hydrogen. Hydroprocessing conversion temperatures are typically 300° to 700° F. Pressures are typically 40 to 100 atm. The reaction times are typically on the order of 10 to 60 minutes. Solid catalysts are employed to increase certain reaction rates, improve selectivity for certain products, and optimize hydrogen consumption.

Suitable methods for the deoxygenation of an oil includes heating an oil to a temperature in the range of from about 350° F. to about 550° F. and continuously contacting the heated oil with nitrogen under at least pressure ranging from about atmospheric to above for at least about 5 minutes.

Suitable methods for isomerization include using alkali isomerization and other oil isomerization known in the art.

Hydrotreating and hydroprocessing ultimately lead to a reduction in the molecular weight of the triglyceride feed. The triglyceride molecule is reduced to four hydrocarbon molecules under hydroprocessing conditions: a propane molecule and three heavier hydrocarbon molecules, typically in the C8 to C18 range.

Thus, in one embodiment, the product of one or more chemical reaction(s) performed on lipid compositions of the invention is an alkane mixture that comprises ASTM D975 renewable diesel. Production of hydrocarbons by oleaginous yeasts is reviewed by Metzger et al. Appl Microbiol Biotechnol (2005) 66: 486-496 and A Look Back at the U.S. Department of Energy's Aquatic Species Program: Biodiesel from Algae, NREL/TP-580-24190, John Sheehan, Terri Dunahay, John Benemann and Paul Roessler (1998).

The distillation properties of a diesel fuel is described in terms of T10-T90 (temperature at 10% and 90%, respectively, volume distilled). Methods of hydrotreating, isomerization, and other covalent modification of oils disclosed herein, as well as methods of distillation and fractionation (such as cold filtration) disclosed herein, can be employed to generate renewable diesel compositions with other T10-T90 ranges, such as 20, 25, 30, 35, 40, 45, 50, 60 and 65° C. using triglyceride oils produced according to the methods disclosed herein.

Methods of hydrotreating, isomerization, and other covalent modification of oils disclosed herein, as well as methods of distillation and fractionation (such as cold filtration) disclosed herein, can be employed to generate renewable diesel compositions with other T10 values, such as T10 between 180 and 295, between 190 and 270, between 210 and 250, between 225 and 245, and at least 290.

Methods of hydrotreating, isomerization, and other covalent modification of oils disclosed herein, as well as methods of distillation and fractionation (such as cold filtration) disclosed herein can be employed to generate renewable diesel compositions with other T90 values, such as T90 between 280 and 380, between 290 and 360, between 300 and 350, between 310 and 340, and at least 290.

Methods of hydrotreating, isomerization, and other covalent modification of oils disclosed herein, as well as methods of distillation and fractionation (such as cold filtration) disclosed herein, can be employed to generate renewable diesel compositions with other FBP values, such as FBP between 290 and 400, between 300 and 385, between 310 and 370, between 315 and 360, and at least 300.

Other oils provided by the methods and compositions of the invention can be subjected to combinations of hydrotreating, isomerization, and other covalent modification including oils with lipid profiles including (a) at least 4% C8-C14; (b) at least 0.3% C8; (c) at least 2% C10; (d) at least 2% C12; and (3) at least 30% C8-C14.

A traditional ultra-low sulfur diesel can be produced from any form of biomass by a two-step process. First, the biomass is converted to a syngas, a gaseous mixture rich in hydrogen and carbon monoxide. Then, the syngas is catalytically converted to liquids. Typically, the production of liquids is accomplished using Fischer-Tropsch (FT) synthesis. This technology applies to coal, natural gas, and heavy oils. Thus, in yet another preferred embodiment of the method for producing renewable diesel, treating the lipid composition to produce an alkane is performed by indirect liquefaction of the lipid composition.

The present invention also provides methods to produce jet fuel. Jet fuel is clear to straw colored. The most common fuel is an unleaded/paraffin oil-based fuel classified as Aeroplane A-1, which is produced to an internationally standardized set of specifications. Jet fuel is a mixture of a large number of different hydrocarbons, possibly as many as a thousand or more. The range of their sizes (molecular weights or carbon numbers) is restricted by the requirements for the product, for example, freezing point or smoke point. Kerosone-type Aeroplane fuel (including Jet A and Jet A-1) has a carbon number distribution between about 8 and 16 carbon numbers. Wide-cut or naphta-type Aeroplane fuel (including Jet B) typically has a carbon number distribution between about 5 and 15 carbons.

Both Aeroplanes (Jet A and Jet B) may contain a number of additives. Useful additives include, but are not limited to, antioxidants, antistatic agents, corrosion inhibitors, and fuel system icing inhibitor (FSII) agents. Antioxidants prevent gumming and usually, are based on alkylated phenols, for example, AO-30, AO-31, or AO-37. Antistatic agents dissipate static electricity and prevent sparking. Stadis 450 with dinonylnaphthylsulfonic acid (DINNSA) as the active ingredient, is an example. Corrosion inhibitors, e.g., DCI-4A is used for civilian and military fuels and DCI-6A is used for military fuels. FSII agents, include, e.g., Di-EGME.

In one embodiment of the invention, a jet fuel is produced by blending oleaginous yeast-derived fuels with existing jet fuel. The lipids produced by the methods of the present invention can serve as feedstock to produce jet fuel. Thus, in another aspect of the present invention, a method for producing jet fuel is provided. Herewith two methods for producing jet fuel from the lipids produced by the methods of the present invention are provided: fluid catalytic cracking (FCC); and hydrodeoxygenation (HDO).

Fluid Catalytic Cracking (FCC) is one method which is used to produce olefins, especially propylene from heavy crude fractions. The lipids produced by the method of the present invention can be converted to olefins. The process involves flowing the lipids produced through an FCC zone and collecting a product stream comprised of olefins, which is useful as a jet fuel. The lipids produced are contacted with a cracking catalyst at cracking conditions to provide a product stream comprising olefins and hydrocarbons useful as jet fuel.

In one embodiment, the method for producing jet fuel comprises (a) cultivating a lipid-containing oleaginous yeast using methods disclosed herein, (b) lysing the lipid-containing oleaginous yeast to produce a lysate, (c) isolating lipid from the lysate, and (d) treating the lipid composition, whereby jet fuel is produced. In one embodiment of the method for producing a jet fuel, the lipid composition can be flowed through a fluid catalytic cracking zone, which, in one embodiment, may comprise contacting the lipid composition with a cracking catalyst at cracking conditions to provide a product stream comprising C₂-C₅ olefins.

In certain embodiments of this method, it may be desirable to remove any contaminants that may be present in the lipid composition. Thus, prior to flowing the lipid composition through a fluid catalytic cracking zone, the lipid composition is pretreated. Pretreatment may involve contacting the lipid composition with an ion-exchange resin. The ion exchange resin is an acidic ion exchange resin, such as Amberlyst™-15 and can be used as a bed in a reactor through which the lipid composition is flowed, either upflow or downflow. Other pretreatments may include mild acid washes by contacting the lipid composition with an acid, such as sulfuric, acetic, nitric, or hydrochloric acid. Contacting is done with a dilute acid solution usually at ambient temperature and atmospheric pressure.

The lipid composition, optionally pretreated, is flowed to an FCC zone where the hydrocarbonaceous components are cracked to olefins. Catalytic cracking is accomplished by contacting the lipid composition in a reaction zone with a catalyst composed of finely divided particulate material. The reaction is catalytic cracking, as opposed to hydrocracking, and is carried out in the absence of added hydrogen or the consumption of hydrogen. As the cracking reaction proceeds, substantial amounts of coke are deposited on the catalyst. The catalyst is regenerated at high temperatures by burning coke from the catalyst in a regeneration zone. Coke-containing catalyst, referred to herein as “coked catalyst”, is continually transported from the reaction zone to the regeneration zone to be regenerated and replaced by essentially coke-free regenerated catalyst from the regeneration zone. Fluidization of the catalyst particles by various gaseous streams allows the transport of catalyst between the reaction zone and regeneration zone. Methods for cracking hydrocarbons, such as those of the lipid composition described herein, in a fluidized stream of catalyst, transporting catalyst between reaction and regeneration zones, and combusting coke in the regenerator are well known by those skilled in the art of FCC processes. Exemplary FCC applications and catalysts useful for cracking the lipid composition to produce C₂-C₅ olefins are described in U.S. Pat. Nos. 6,538,169, 7,288,685, which are incorporated in their entirety by reference.

Suitable FCC catalysts generally comprise at least two components that may or may not be on the same matrix. In some embodiments, both two components may be circulated throughout the entire reaction vessel. The first component generally includes any of the well-known catalysts that are used in the art of fluidized catalytic cracking, such as an active amorphous clay-type catalyst and/or a high activity, crystalline molecular sieve. Molecular sieve catalysts may be preferred over amorphous catalysts because of their much-improved selectivity to desired products. In some preferred embodiments, zeolites may be used as the molecular sieve in the FCC processes. Preferably, the first catalyst component comprises a large pore zeolite, such as an Y-type zeolite, an active alumina material, a binder material, comprising either silica or alumina and an inert filler such as kaolin.

In one embodiment, cracking the lipid composition of the present invention, takes place in the riser section or, alternatively, the lift section, of the FCC zone. The lipid composition is introduced into the riser by a nozzle resulting in the rapid vaporization of the lipid composition. Before contacting the catalyst, the lipid composition will ordinarily have a temperature of about 149° C. to about 316° C. (300° F. to 600° F.). The catalyst is flowed from a blending vessel to the riser where it contacts the lipid composition for a time of abort 2 seconds or less.

The blended catalyst and reacted lipid composition vapors are then discharged from the top of the riser through an outlet and separated into a cracked product vapor stream including olefins and a collection of catalyst particles covered with substantial quantities of coke and generally referred to as “coked catalyst.” In an effort to minimize the contact time of the lipid composition and the catalyst which may promote further conversion of desired products to undesirable other products, any arrangement of separators such as a swirl arm arrangement can be used to remove coked catalyst from the product stream quickly. The separator, e.g. swirl arm separator, is located in an upper portion of a chamber with a stripping zone situated in the lower portion of the chamber. Catalyst separated by the swirl arm arrangement drops down into the stripping zone. The cracked product vapor stream comprising cracked hydrocarbons including light olefins and some catalyst exit the chamber via a conduit which is in communication with cyclones. The cyclones remove remaining catalyst particles from the product vapor stream to reduce particle concentrations to very low levels. The product vapor stream then exits the top of the separating vessel. Catalyst separated by the cyclones is returned to the separating vessel and then to the stripping zone. The stripping zone removes adsorbed hydrocarbons from the surface of the catalyst by counter-current contact with steam.

Low hydrocarbon partial pressure operates to favor the production of light olefins. Accordingly, the riser pressure is set at about 172 to 241 kPa (25 to 35 psia) with a hydrocarbon partial pressure of about 35 to 172 kPa (5 to 25 psia), with a preferred hydrocarbon partial pressure of about 69 to 138 kPa (10 to 20 psia). This relatively low partial pressure for hydrocarbon is achieved by using steam as a diluent to the extent that the diluent is 10 to 55 wt-% of lipid composition and preferably about 15 wt-% of lipid composition. Other diluents such as dry gas can be used to reach equivalent hydrocarbon partial pressures.

The temperature of the cracked stream at the riser outlet will be about 510° C. to 621° C. (950° F. to 1150° F.). However, riser outlet temperatures above 566° C. (1050° F.) make more dry gas and more olefins. Whereas, riser outlet temperatures below 566° C. (1050° F.) make less ethylene and propylene. Accordingly, it is preferred to run the FCC process at a preferred temperature of about 566° C. to about 630° C., preferred pressure of about 138 kPa to about 240 kPa (20 to 35 psia). Another condition for the process is the catalyst to lipid composition ratio which can vary from about 5 to about 20 and preferably from about 10 to about 15.

In one embodiment of the method for producing a jet fuel, the lipid composition is introduced into the lift section of an FCC reactor. The temperature in the lift section will be very hot and range from about 700° C. (1292° F.) to about 760° C. (1400° F.) with a catalyst to lipid composition ratio of about 100 to about 150. It is anticipated that introducing the lipid composition into the lift section will produce considerable amounts of propylene and ethylene.

In another embodiment of the method for producing a jet fuel using the lipid composition or the lipids produced as described herein, the structure of the lipid composition or the lipids is broken by a process referred to as hydrodeoxygenation (HDO). HDO means removal of oxygen by means of hydrogen, that is, oxygen is removed while breaking the structure of the material. Olefinic double bonds are hydrogenated and any sulphur and nitrogen compounds are removed. Sulphur removal is called hydrodesulphurization (HDS). Pretreatment and purity of the raw materials (lipid composition or the lipids) contribute to the service life of the catalyst.

Generally in the HDO/HDS step, hydrogen is mixed with the feed stock (lipid composition or the lipids) and then the mixture is passed through a catalyst bed as a co-current flow, either as a single phase or a two phase feed stock. After the HDO/MDS step, the product fraction is separated and passed to a separate isomerization reactor. An isomerization reactor for biological starting material is described in the literature (FI 100 248) as a co-current reactor.

The process for producing a fuel by hydrogenating a hydrocarbon feed, e.g., the lipid composition or the lipids herein, can also be performed by passing the lipid composition or the lipids as a co-current flow with hydrogen gas through a first hydrogenation zone, and thereafter the hydrocarbon effluent is further hydrogenated in a second hydrogenation zone by passing hydrogen gas to the second hydrogenation zone as a counter-current flow relative to the hydrocarbon effluent. Exemplary HDO applications and catalysts useful for cracking the lipid composition to produce C₂-C₅ olefins are described in U.S. Pat. No. 7,232,935, which is incorporated in its entirety by reference.

Typically, in the hydrodeoxygenation step, the structure of the biological component, such as the lipid composition or lipids herein, is decomposed, oxygen, nitrogen, phosphorus and sulphur compounds, and light hydrocarbons as gas are removed, and the olefinic bonds are hydrogenated. In the second step of the process, i.e. in the so-called isomerization step, isomerization is carried out for branching the hydrocarbon chain and improving the performance of the paraffin at low temperatures.

In the first step, i.e. HDO step, of the cracking process, hydrogen gas and the lipid composition or lipids herein which are to be hydrogenated are passed to a HDO catalyst bed system either as co-current or counter-current flows, said catalyst bed system comprising one or more catalyst bed(s), preferably 1-3 catalyst beds. The HDO step is typically operated in a co-current manner. In case of a HDO catalyst bed system comprising two or more catalyst beds, one or more of the beds may be operated using the counter-current flow principle. In the HDO step, the pressure varies between 20 and 150 bar, preferably between 50 and 100 bar, and the temperature varies between 200 and 500° C., preferably in the range of 300-400° C. In the HDO step, known hydrogenation catalysts containing metals from Group VII and/or VIB of the Periodic System may be used. Preferably, the hydrogenation catalysts are supported Pd, Pt, Ni, NiMo or a CoMo catalysts, the support being alumina and/or silica. Typically, NiMo/Al₂O₃ and CoMo/Al₂O₃ catalysts are used.

Prior to the HDO step, the lipid composition or lipids herein may optionally be treated by prehydrogenation under milder conditions thus avoiding side reactions of the double bonds. Such prehydrogenation is carried out in the presence of a prehydrogenation catalyst at temperatures of 50 400° C. and at hydrogen pressures of 1 200 bar, preferably at a temperature between 150 and 250° C. and at a hydrogen pressure between 10 and 100 bar. The catalyst may contain metals from Group VIII and/or VIB of the Periodic System. Preferably, the prehydrogenation catalyst is a supported Pd, Pt, Ni, NiMo or a CoMo catalyst, the support being alumina and/or silica.

A gaseous stream from the HDO step containing hydrogen is cooled and then carbon monoxide, carbon dioxide, nitrogen, phosphorus and sulphur compounds, gaseous light hydrocarbons and other impurities are removed therefrom. After compressing, the purified hydrogen or recycled hydrogen is returned back to the first catalyst bed and/or between the catalyst beds to make up for the withdrawn gas stream. Water is removed from the condensed liquid. The liquid is passed to the first catalyst bed or between the catalyst beds.

After the HDO step, the product is subjected to an isomerization step. It is substantial for the process that the impurities are removed as completely as possible before the hydrocarbons are contacted with the isomerization catalyst. The isomerization step comprises an optional stripping step, wherein the reaction product from the HDO step may be purified by stripping with water vapour or a suitable gas such as light hydrocarbon, nitrogen or hydrogen. The optional stripping step is carried out in counter-current manner in a unit upstream of the isomerization catalyst, wherein the gas and liquid are contacted with each other, or before the actual isomerization reactor in a separate stripping unit utilizing counter-current principle.

After the stripping step the hydrogen gas and the hydrogenated lipid composition or lipids herein, and optionally an n-paraffin mixture, are passed to a reactive isomerization unit comprising one or several catalyst bed(s). The catalyst beds of the isomerization step may operate either in co-current or counter-current manner.

It is important for the process that the counter-current flow principle is applied in the isomerization step. In the isomerization step this is done by carrying out either the optional stripping step or the isomerization reaction step or both in counter-current manner. In the isomerization step, the pressure varies in the range of 20 150 bar, preferably in the range of 20 100 bar, the temperature being between 200 and 500° C., preferably between 300 and 400° C. In the isomerization step, isomerization catalysts known in the art may be used. Suitable isomerization catalysts contain molecular sieve and/or a metal from Group VII and/or a carrier. Preferably, the isomerization catalyst contains SAPO-11 or SAPO41 or ZSM-22 or ZSM-23 or ferrierite and Pt, Pd or Ni and Al₂O₃ or SiO₂. Typical isomerization catalysts are, for example, Pt/SAPO-11/Al₂O₃, Pt/ZSM-22/Al₂O₃, Pt/ZSM-23/Al₂O₃ and Pt/SAPO-11/SiO₂. The isomerization step and the HDO step may be carried out in the same pressure vessel or in separate pressure vessels. Optional prehydrogenation may be carried out in a separate pressure vessel or in the same pressure vessel as the HDO and isomerization steps.

Thus, in one embodiment, the product of the one or more chemical reactions is an alkane mixture that comprises ASTM D1655 jet fuel. In some embodiments, the composition conforming to the specification of ASTM 1655 jet fuel has a sulfur content that is less than 10 ppm. In other embodiments, the composition conforming to the specification of ASTM 1655 jet fuel has a T10 value of the distillation curve of less than 205° C. In another embodiment, the composition conforming to the specification of ASTM 1655 jet fuel has a final boiling point (FBP) of less than 300° C. In another embodiment, the composition conforming to the specification of ASTM 1655 jet fuel has a flash point of at least 38° C. In another embodiment, the composition conforming to the specification of ASTM 1655 jet fuel has a density between 775K/M³ and 840K/M³. In yet another embodiment, the composition conforming to the specification of ASTM 1655 jet fuel has a freezing point that is below −47° C. In another embodiment, the composition conforming to the specification of ASTM 1655 jet fuel has a net Heat of Combustion that is at least 42.8 MJ/K. In another embodiment, the composition conforming to the specification of ASTM 1655 jet fuel has a hydrogen content that is at least 13.4 mass %. In another embodiment, the composition conforming to the specification of ASTM 1655 jet fuel has a thermal stability, as tested by quantitative gravimetric JFTOT at 260° C., that is below 3 mm of Hg. In another embodiment, the composition conforming to the specification of ASTM 1655 jet fuel has an existent gum that is below 7 mg/dl.

Thus, the present invention discloses a variety of methods in which chemical modification of oleaginous yeast lipid is undertaken to yield products useful in a variety of industrial and other applications. Examples of processes for modifying oil produced by the methods disclosed herein include, but are not limited to, hydrolysis of the oil, hydroprocessing of the oil, and esterification of the oil. The modification of the oleaginous yeast oil produces basic oleochemicals that can be further modified into selected derivative oleochemicals for a desired function. In a manner similar to that described above with reference to fuel producing processes, these chemical modifications can also be performed on oils generated from the oleaginous yeast cultures described herein. Examples of basic oleochemicals include, but are not limited to, soaps, fatty acids, fatty acid methyl esters, and glycerol. Examples of derivative oleochemicals include, but are not limited to, fatty nitriles, esters, dimer acids, quats, surfactants, fatty alkanolamides, fatty alcohol sulfates, resins, emulsifiers, fatty alcohols, olefins, and higher alkanes.

Hydrolysis of the fatty acid constituents from the glycerolipids produced by the methods of the invention yields free fatty acids that can be derivatized to produce other useful chemicals. Hydrolysis occurs in the presence of water and a catalyst which may be either an acid or a base. The liberated free fatty acids can be derivatized to yield a variety of products, as reported in the following: U.S. Pat. Nos. 5,304,664 (Highly sulfated fatty acids); 7,262,158 (Cleansing compositions); 7,115,173 (Fabric softener compositions); 6,342,208 (Emulsions for treating skin); 7,264,886 (Water repellant compositions); 6,924,333 (Paint additives); 6,596,768 (Lipid-enriched ruminant feedstock); and 6,380,410 (Surfactants for detergents and cleaners).

With regard to hydrolysis, in one embodiment of the invention, a triglyceride oil is optionally first hydrolyzed in a liquid medium such as water or sodium hydroxide so as to obtain glycerol and soaps. There are various suitable triglyceride hydrolysis methods, including, but not limited to, saponification, acid hydrolysis, alkaline hydrolysis, enzymatic hydrolysis (referred herein as splitting), and hydrolysis using hot-compressed water. One skilled in the art will recognize that a triglyceride oil need not be hydrolyzed in order to produce an oleochemical; rather, the oil may be converted directly to the desired oleochemical by other known process. For example, the triglyceride oil may be directly converted to a methyl ester fatty acid through esterification.

In some embodiments, catalytic hydrolysis of the oil produced by methods disclosed herein occurs by splitting the oil into glycerol and fatty acids. As discussed above, the fatty acids may then be further processed through several other modifications to obtained derivative oleochemicals. For example, in one embodiment the fatty acids may undergo an amination reaction to produce fatty nitrogen compounds. In another embodiment, the fatty acids may undergo ozonolysis to produce mono- and dibasic-acids.

In other embodiments hydrolysis may occur via the, splitting of oils produced herein to create oleochemicals. In some preferred embodiments of the invention, a triglyceride oil may be split before other processes is performed. One skilled in the art will recognize that there are many suitable triglyceride splitting methods, including, but not limited to, enzymatic splitting and pressure splitting.

Generally, enzymatic oil splitting methods use enzymes, lipases, as biocatalysts acting on a water/oil mixture. Enzymatic splitting then splits the oil or fat, respectively, is into glycerol and free fatty acids. The glycerol may then migrates into the water phase whereas the organic phase enriches with free fatty acids.

The enzymatic splitting reactions generally take place at the phase boundary between organic and aqueous phase, where the enzyme is present only at the phase boundary. Triglycerides that meet the phase boundary then contribute to or participate in the splitting reaction. As the reaction proceeds, the occupation density or concentration of fatty acids still chemically bonded as glycerides, in comparison to free fatty acids, decreases at the phase boundary so that the reaction is slowed down. In certain embodiments, enzymatic splitting may occur at room temperature. One of ordinary skill in the art would know the suitable conditions for splitting oil into the desired fatty acids.

By way of example, the reaction speed can be accelerated by increasing the interface boundary surface. Once the reaction is complete, free fatty acids are then separated from the organic phase freed from enzyme, and the residue which still contains fatty acids chemically bonded as glycerides is fed back or recycled and mixed with fresh oil or fat to be subjected to splitting. In this manner, recycled glycerides are then subjected to a further enzymatic splitting process. In some embodiments, the free fatty acids are extracted from an oil or fat partially split in such a manner. In that way, if the chemically bound fatty acids (triglycerides) are returned or fed back into the splitting process, the enzyme consumption can be drastically reduced.

The splitting degree is determined as the ratio of the measured acid value divided by the theoretically possible acid value which can be computed for a given oil or fat. Preferably, the acid value is measured by means of titration according to standard common methods. Alternatively, the density of the aqueous glycerol phase can be taken as a measure for the splitting degree.

In one embodiment, the slitting process as described herein is also suitable for splitting the mono-, di- and triglyceride that are contained in the so-called soap-stock from the alkali refining processes of the produced oils. In this manner, the soap-stock can be quantitatively converted without prior saponification of the neutral oils into the fatty acids. For this purpose, the fatty acids being chemically bonded in the soaps are released, preferably before splitting, through an addition of acid. In certain embodiments, a buffer solution is used in addition to water and enzyme for the splitting process.

In one embodiment, oils produced in accordance with the methods of the invention can also be subjected to saponification as a method of hydrolysis. Animal and plant oils are typically made of triacylglycerols (TAGs), which are esters of fatty acids with the trihydric alcohol, glycerol. In an alkaline hydrolysis reaction, the glycerol in a TAG is removed, leaving three carboxylic acid anions that can associate with alkali metal cations such as sodium or potassium to produce fatty acid salts. In this scheme, the carboxylic acid constituents are cleaved from the glycerol moiety and replaced with hydroxyl groups. The quantity of base (e.g., KOH) that is used in the reaction is determined by the desired degree of saponification. If the objective is, for example, to produce a soap product that comprises some of the oils originally present in the TAG composition, an amount of base insufficient to convert all of the TAGs to fatty acid salts is introduced into the reaction mixture. Normally, this reaction is performed in an aqueous solution and proceeds slowly, but may be expedited by the addition of heat. Precipitation of the fatty acid salts can be facilitated by addition of salts, such as water-soluble alkali metal halides (e.g., NaCl or KCl), to the reaction mixture. Preferably, the base is an alkali metal hydroxide, such as NaOH or KOH. Alternatively, other bases, such as alkanolamines, including for example triethanolamine and aminomethylpropanol, can be used in the reaction scheme. In some cases, these alternatives may be preferred to produce a clear soap product.

In some methods, the first step of chemical modification may be hydroprocessing to saturate double bonds, followed by deoxygenation at elevated temperature in the presence of hydrogen and a catalyst. In other methods, hydrogenation and deoxygenation may occur in the same reaction. In still other methods deoxygenation occurs before hydrogenation. Isomerization may then be optionally performed, also in the presence of hydrogen and a catalyst. Finally, gases and naphtha components can be removed if desired. For example, see U.S. Pat. Nos. 5,475,160 (hydrogenation of triglycerides); 5,091,116 (deoxygenation, hydrogenation and gas removal); 6,391,815 (hydrogenation); and 5,888,947 (isomerization).

In some embodiments of the invention, the triglyceride oils are partially or completely deoxygenated. The deoxygenation reactions form desired products, including, but not limited to, fatty acids, fatty alcohols, polyols, ketones, and aldehydes. In general, without being limited by any particular theory, the deoxygenation reactions involve a combination of various different reaction pathways, including without limitation: hydrogenolysis, hydrogenation, consecutive hydrogenation-hydrogenolysis, consecutive hydrogenolysis-hydrogenation, and combined hydrogenation-hydrogenolysis reactions, resulting in at least the partial removal of oxygen from the fatty acid or fatty acid ester to produce reaction products, such as fatty alcohols, that can be easily converted to the desired chemicals by further processing. For example, in one embodiment, a fatty alcohol may be converted to olefins through FCC reaction or to higher alkanes through a condensation reaction.

One such chemical modification is hydrogenation, which is the addition of hydrogen to double bonds in the fatty acid constituents of glycerolipids or of free fatty acids. The hydrogenation process permits the transformation of liquid oils into semi-solid or solid fats, which may be more suitable for specific applications.

Hydrogenation of oil produced by the methods described herein can be performed in conjunction with one or more of the methods and/or materials provided herein, as reported in the following: U.S. Pat. Nos. 7,288,278 (Food additives or medicaments); 5,346,724 (Lubrication products); 5,475,160 (Fatty alcohols); 5,091,116 (Edible oils); 6,808,737 (Structural fats for margarine and spreads); 5,298,637 (Reduced-calorie fat substitutes); 6,391,815 (Hydrogenation catalyst and sulfur adsorbent); 5,233,099 and 5,233,100 (Fatty alcohols); 4,584,139 (Hydrogenation catalysts); 6,057,375 (Foam suppressing agents); and 7,118,773 (Edible emulsion spreads).

One skilled in the art will recognize that various processes may be used to hydrogenate carbohydrates. One suitable method includes contacting the carbohydrate with hydrogen or hydrogen mixed with a suitable gas and a catalyst under conditions sufficient in a hydrogenation reactor to form a hydrogenated product. The hydrogenation catalyst generally can include Cu, Re, Ni, Fe, Co, Ru, Pd, Rh, Pt, Os, Ir, and alloys or any combination thereof, either alone or with promoters such as W, Mo, Au, Ag, Cr, Zn, Mn, Sn, B, P, Bi, and alloys or any combination thereof. Other effective hydrogenation catalyst materials include either supported nickel or ruthenium modified with rhenium. In an embodiment, the hydrogenation catalyst also includes any one of the supports, depending on the desired functionality of the catalyst. The hydrogenation catalysts may be prepared by methods known to those of ordinary skill in the art.

In some embodiments the hydrogenation catalyst includes a supported Group VIII metal catalyst and a metal sponge material (e.g., a sponge nickel catalyst). Raney nickel provides an example of an activated sponge nickel catalyst suitable for use in this invention. In other embodiment, the hydrogenation reaction in the invention is performed using a catalyst comprising a nickel-rhenium catalyst or a tungsten-modified nickel catalyst. One example of a suitable catalyst for the hydrogenation reaction of the invention is a carbon-supported nickel-rhenium catalyst.

In an embodiment, a suitable Raney nickel catalyst may be prepared by treating an alloy of approximately equal amounts by weight of nickel and aluminum with an aqueous alkali solution, e.g., containing about 25 weight % of sodium hydroxide. The aluminum is selectively dissolved by the aqueous alkali solution resulting in a sponge shaped material comprising mostly nickel with minor amounts of aluminum. The initial alloy includes promoter metals (i.e., molybdenum or chromium) in the amount such that about 1 to 2 weight % remains in the formed sponge nickel catalyst. In another embodiment, the hydrogenation catalyst is prepared using a solution of ruthenium(III) nitrosylnitrate, ruthenium (III) chloride in water to impregnate a suitable support material. The solution is then dried to form a solid having a water content of less than about 1% by weight. The solid may then be reduced at atmospheric pressure in a hydrogen stream at 300° C. (uncalcined) or 400° C. (calcined) in a rotary ball furnace for 4 hours. After cooling and rendering the catalyst inert with nitrogen, 5% by volume of oxygen in nitrogen is passed over the catalyst for 2 hours.

In certain embodiments, the catalyst described includes a catalyst support. The catalyst support stabilizes and supports the catalyst. The type of catalyst support used depends on the chosen catalyst and the reaction conditions. Suitable supports for the invention include, but are not limited to, carbon, silica, silica-alumina, zirconia, titania, ceria, vanadia, nitride, boron nitride, heteropolyacids, hydroxyapatite, zinc oxide, chromia, zeolites, carbon nanotubes, carbon fullerene and any combination thereof.

The catalysts used in this invention can be prepared using conventional methods known to those in the art. Suitable methods may include, but are not limited to, incipient wetting, evaporative impregnation, chemical vapor deposition, wash-coating, magnetron sputtering techniques, and the like.

The conditions for which to carry out the hydrogenation reaction will vary based on the type of starting material and the desired products. One of ordinary skill in the art, with the benefit of this disclosure, will recognize the appropriate reaction conditions. In general, the hydrogenation reaction is conducted at temperatures of 80° C. to 250° C., and preferably at 90° C. to 200° C., and most preferably at 100° C. to 150° C. In some embodiments, the hydrogenation reaction is conducted at pressures from 500 KPa to 14000 KPa.

The hydrogen used in the hydrogenolysis reaction of the current invention may include external hydrogen, recycled hydrogen, in situ generated hydrogen, and any combination thereof. As used herein, the term “external hydrogen” refers to hydrogen that does not originate from the biomass reaction itself, but rather is added to the system from another source.

In some embodiments of the invention, it is desirable to convert the starting carbohydrate to a smaller molecule that will be more readily converted to desired higher hydrocarbons. One suitable method for this conversion is through a hydrogenolysis reaction. Various processes are known for performing hydrogenolysis of carbohydrates. One suitable method includes contacting a carbohydrate with hydrogen or hydrogen mixed with a suitable gas and a hydrogenolysis catalyst in a hydrogenolysis reactor under conditions sufficient to form a reaction product comprising smaller molecules or polyols. As used herein, the term “smaller molecules or polyols” includes any molecule that has a smaller molecular weight, which can include a smaller number of carbon atoms or oxygen atoms than the starting carbohydrate. In an embodiment, the reaction products include smaller molecules that include polyols and alcohols. Someone of ordinary skill in the art would be able to choose the appropriate method by which to carry out the hydrogenolysis reaction.

In some embodiments, a 5 and/or 6 carbon sugar or sugar alcohol may be converted to propylene glycol, ethylene glycol, and glycerol using a hydrogenolysis catalyst. The hydrogenolysis catalyst may include Cr, Mo, W, Re, Mn, Cu, Cd, Fe, Co, Ni, Pt, Pd, Rh, Ru, Ir, Os, and alloys or any combination thereof, either alone or with promoters such as Au, Ag, Cr, Zn, Mn, Sn, Bi, B, O, and alloys or any combination thereof. The hydrogenolysis catalyst may also include a carbonaceous pyropolymer catalyst containing transition metals (e.g., chromium, molybdenum, tungsten, rhenium, manganese, copper, cadmium) or Group VIII metals (e.g., iron, cobalt, nickel, platinum, palladium, rhodium, ruthenium, iridium, and osmium). In certain embodiments, the hydrogenolysis catalyst may include any of the above metals combined with an alkaline earth metal oxide or adhered to a catalytically active support. In certain embodiments, the catalyst described in the hydrogenolysis reaction may include a catalyst support as described above for the hydrogenation reaction.

The conditions for which to carry out the hydrogenolysis reaction will vary based on the type of starting material and the desired products. One of ordinary skill in the art, with the benefit of this disclosure, will recognize the appropriate conditions to use to carry out the reaction. In general, they hydrogenolysis reaction is conducted at temperatures of 110° C. to 300° C., and preferably at 170° C. to 220° C., and most preferably at 200° C. to 225° C. In some embodiments, the hydrogenolysis reaction is conducted under basic conditions, preferably at a pH of 8 to 13, and even more preferably at a pH of 10 to 12. In some embodiments, the hydrogenolysis reaction is conducted at pressures in a range between 60 KPa and 16500 KPa, and preferably in a range between 1700 KPa and 14000 KPa, and even more preferably between 4800 KPa and 11000 KPa.

The hydrogen used in the hydrogenolysis reaction of the current invention can include external hydrogen, recycled hydrogen, in situ generated hydrogen, and any combination thereof.

In some embodiments, the reaction products discussed above may be converted into higher hydrocarbons through a condensation reaction in a condensation reactor. In such embodiments, condensation of the reaction products occurs in the presence of a catalyst capable of forming higher hydrocarbons. While not intending to be limited by theory, it is believed that the production of higher hydrocarbons proceeds through a stepwise addition reaction including the formation of carbon-carbon, or carbon-oxygen bond. The resulting reaction products include any number of compounds containing these moieties, as described in more detail below.

In certain embodiments, suitable condensation catalysts include an acid catalyst, a base catalyst, or an acid/base catalyst. As used herein, the term “acid/base catalyst” refers to a catalyst that has both an acid and a base functionality. In some embodiments the condensation catalyst can include, without limitation, zeolites, carbides, nitrides, zirconia, alumina, silica, aluminosilicates, phosphates, titanium oxides, zinc oxides, vanadium oxides, lanthanum oxides, yttrium oxides, scandium oxides, magnesium oxides, cerium oxides, barium oxides, calcium oxides, hydroxides, heteropolyacids, inorganic acids, acid modified resins, base modified resins, and any combination thereof. In some embodiments, the condensation catalyst can also include a modifier. Suitable modifiers include La, Y, Sc, P, B, Bi, Li, Na, K, Rb, Cs, Mg, Ca, Sr, Ba, and any combination thereof. In some embodiments, the condensation catalyst can also include a metal. Suitable metals include Cu, Ag, Au, Pt, Ni, Fe, Co, Ru, Zn, Cd, Ga, In, Rh, Pd, Ir, Re, Mn, Cr, Mo, W, Sn, Os, alloys, and any combination thereof.

In certain embodiments, the catalyst described in the condensation reaction may include a catalyst support as described above for the hydrogenation reaction. In certain embodiments, the condensation catalyst is self-supporting. As used herein, the term “self-supporting” means that the catalyst does not need another material to serve as support. In other embodiments, the condensation catalyst in used in conjunction with a separate support suitable for suspending the catalyst. In an embodiment, the condensation catalyst support is silica.

The conditions under which the condensation reaction occurs will vary based on the type of starting material and the desired products. One of ordinary skill in the art, with the benefit of this disclosure, will recognize the appropriate conditions to use to carry out the reaction. In some embodiments, the condensation reaction is carried out at a temperature at which the thermodynamics for the proposed reaction are favorable. The temperature for the condensation reaction will vary depending on the specific starting polyol or alcohol. In some embodiments, the temperature for the condensation reaction is in a range from 80° C. to 500° C., and preferably from 125° C. to 450° C., and most preferably from 125° C. to 250° C. In some embodiments, the condensation reaction is conducted at pressures in a range between 0 Kpa to 9000 KPa, and preferably in a range between 0 KPa and 7000 KPa, and even more preferably between 0 KPa and 5000 KPa.

The higher alkanes formed by the invention include, but are not limited to, branched or straight chain alkanes that have from 4 to 30 carbon atoms, branched or straight chain alkenes that have from 4 to 30 carbon atoms, cycloalkanes that have from 5 to 30 carbon atoms, cycloalkenes that have from 5 to 30 carbon atoms, aryls, fused aryls, alcohols, and ketones. Suitable alkanes include, but are not limited to, butane, pentane, pentene, 2-methylbutane, hexane, hexene, 2-methylpentane, 3-methylpentane, 2,2,-dimethylbutane, 2,3-dimethylbutane, heptane, heptene, octane, octene, 2,2,4-trimethylpentane, 2,3-dimethyl hexane, 2,3,4-trimethylpentane, 2,3-dimethylpentane, nonane, nonene, decane, decene, undecane, undecene, dodecane, dodecene, tridecane, tridecene, tetradecane, tetradecene, pentadecane, pentadecene, nonyldecane, nonyldecene, eicosane, eicosene, uneicosane, uneicosene, doeicosane, doeicosene, trieicosane, trieicosene, tetraeicosane, tetraeicosene, and isomers thereof. Some of these products may be suitable for use as fuels.

In some embodiments, the cycloalkanes and the cycloalkenes are unsubstituted. In other embodiments, the cycloalkanes and cycloalkenes are mono-substituted. In still other embodiments, the cycloalkanes and cycloalkenes are multi-substituted. In the embodiments comprising the substituted cycloalkanes and cycloalkenes, the substituted group includes, without limitation, a branched or straight chain alkyl having 1 to 12 carbon atoms, a branched or straight chain alkylene having 1 to 12 carbon atoms, a phenyl, and any combination thereof. Suitable cycloalkanes and cycloalkenes include, but are not limited to, cyclopentane, cyclopentene, cyclohexane, cyclohexene, methyl-cyclopentane, methyl-cyclopentene, ethyl-cyclopentane, ethyl-cyclopentene, ethyl-cyclohexane, ethyl-cyclohexene, isomers and any combination thereof.

In some embodiments, the aryls formed are unsubstituted. In another embodiment, the aryls formed are mono-substituted. In the embodiments comprising the substituted aryls, the substituted group includes, without limitation, a branched or straight chain alkyl having 1 to 12 carbon atoms, a branched or straight chain alkylene having 1 to 12 carbon atoms, a phenyl, and any combination thereof. Suitable aryls for the invention include, but are not limited to, benzene, toluene, xylene, ethyl benzene, para xylene, meta xylene, and any combination thereof.

The alcohols produced in the invention have from 4 to 30 carbon atoms. In some embodiments, the alcohols are cyclic. In other embodiments, the alcohols are branched. In another embodiment, the alcohols are straight chained. Suitable alcohols for the invention include, but are not limited to, butanol, pentanol, hexanol, heptanol, octanol, nonanol, decanol, undecanol, dodecanol, tridecanol, tetradecanol, pentadecanol, hexadecanol, heptyldecanol, octyldecanol, nonyldecanol, eicosanol, uneicosanol, doeicosanol, trieicosanol, tetraeicosanol, and isomers thereof.

The ketones produced in the invention have from 4 to 30 carbon atoms. In an embodiment, the ketones are cyclic. In another embodiment, the ketones are branched. In another embodiment, the ketones are straight chained. Suitable ketones for the invention include, but are not limited to, butanone, pentanone, hexanone, heptanone, octanone, nonanone, decanone, undecanone, dodecanone, tridecanone, tetradecanone, pentadecanone, hexadecanone, heptyldecanone, octyldecanone, nonyldecanone, eicosanone, uneicosanone, doeicosanone, trieicosanone, tetraeicosanone, and isomers thereof.

Another such chemical modification is interesterification. Naturally produced glycerolipids do not have a uniform distribution of fatty acid constituents. In the context of oils, interesterification refers to the exchange of acyl radicals between two esters of different glycerolipids. The interesterification process provides a mechanism by which the fatty acid constituents of a mixture of glycerolipids can be rearranged to modify the distribution pattern. Interesterification is a well-known chemical process, and generally comprises heating (to about 200° C.) a mixture of oils for a period (e.g, 30 minutes) in the presence of a catalyst, such as an alkali metal or alkali metal alkylate (e.g., sodium methoxide). This process can be used to randomize the distribution pattern of the fatty acid constituents of an oil mixture, or can be directed to produce a desired distribution pattern. This method of chemical modification of lipids can be performed on materials provided herein, such as oleaginous yeast biomass with a percentage of dry cell weight as lipid at least 20%.

Directed interesterification, in which a specific distribution pattern of fatty acids is sought, can be performed by maintaining the oil mixture at a temperature below the melting point of some TAGs which might occur. This results in selective crystallization of these TAGs, which effectively removes them from the reaction mixture as they crystallize. The process can be continued until most of the fatty acids in the oil have precipitated, for example. A directed interesterification process can be used, for example, to produce a product with a lower calorie content via the substitution of longer-chain fatty acids with shorter-chain counterparts. Directed interesterification can also be used to produce a product with a mixture of fats that can provide desired melting characteristics and structural features sought in food additives or products (e.g., margarine) without resorting to hydrogenation, which can produce unwanted trans isomers.

Interesterification of oils produced by the methods described herein can be performed in conjunction with one or more of the methods and/or materials, or to produce products, as reported in the following: U.S. Pat. Nos. 6,080,853 (Nondigestible fat substitutes); 4,288,378 (Peanut butter stabilizer); 5,391,383 (Edible spray oil); 6,022,577 (Edible fats for food products); 5,434,278 (Edible fats for food products); 5,268,192 (Low calorie nut products); 5,258,197 (Reduce calorie edible compositions); 4,335,156 (Edible fat product); 7,288,278 (Food additives or medicaments); 7,115,760 (Fractionation process); 6,808,737 (Structural fats); 5,888,947 (Engine lubricants); 5,686,131 (Edible oil mixtures); and 4,603,188 (Curable urethane compositions).

In one embodiment in accordance with the invention, transesterification of the oil, as described above, is followed by reaction of the transesterified product with polyol, as reported in U.S. Pat. No. 6,465,642, to produce polyol fatty acid polyesters. Such an esterification and separation process may comprise the steps as follows: reacting a lower alkyl ester with polyol in the presence of soap; removing residual soap from the product mixture; water-washing and drying the product mixture to remove impurities; bleaching the product mixture for refinement; separating at least a portion of the unreacted lower alkyl ester from the polyol fatty acid polyester in the product mixture; and recycling the separated unreacted lower alkyl ester.

Transesterification can also be performed on oleaginous yeast biomass with short chain fatty acid esters, as reported in U.S. Pat. No. 6,278,006. In general, transesterification may be performed by adding a short chain fatty acid ester to an oil in the presence of a suitable catalyst and heating the mixture. In some embodiments, the oil comprises about 5% to about 90% of the reaction mixture by weight. In some embodiments, the short chain fatty acid esters can be about 10% to about 50% of the reaction mixture by weight. Non-limiting examples of catalysts include base catalysts, sodium methoxide, acid catalysts including inorganic acids such as sulfuric acid and acidified clays, organic acids such as methane sulfonic acid, benzenesulfonic acid, and toluenesulfonic acid, and acidic resins such as Amberlyst 15. Metals such as sodium and magnesium, and metal hydrides also are useful catalysts.

Another such chemical modification is hydroxylation, which involves the addition of water to a double bond resulting in saturation and the incorporation of a hydroxyl moiety. The hydroxylation process provides a mechanism for converting one or more fatty acid constituents of a glycerolipid to a hydroxy fatty acid. Hydroxylation can be performed, for example, via the method reported in U.S. Pat. No. 5,576,027. Hydroxylated fatty acids, including castor oil and its derivatives, are useful as components in several industrial applications, including food additives, surfactants, pigment wetting agents, defoaming agents, water proofing additives, plasticizing agents, cosmetic emulsifying and/or deodorant agents, as well as in electronics, pharmaceuticals, paints, inks, adhesives, and lubricants. One example of how the hydroxylation of a glyceride may be performed is as follows: fat may be heated, preferably to about 30-50° C. combined with heptane and maintained at temperature for thirty minutes or more; acetic acid may then be added to the mixture followed by an aqueous solution of sulfuric acid followed by an aqueous hydrogen peroxide solution which is added in small increments to the mixture over one hour; after the aqueous hydrogen peroxide, the temperature may then be increased to at least about 60° C. and stirred for at least six hours; after the stirring, the mixture is allowed to settle and a lower aqueous layer formed by the reaction may be removed while the upper heptane layer formed by the reaction may be washed with hot water having a temperature of about 60° C.; the washed heptane layer may/then be neutralized with an aqueous potassium hydroxide solution to a pH of about 5 to 7 and then removed by distillation under vacuum; the reaction product may then be dried under vacuum at 100° C. and the dried product steam-deodorized under vacuum conditions and filtered at about 50° to 60° C. using diatomaceous earth.

Hydroxylation of oleaginous yeast oils produced by the methods described herein can be performed in conjunction with one or more of the methods and/or materials, or to produce products, as reported in the following: U.S. Pat. Nos. 6,590,113 (Oil-based coatings and ink); 4,049,724 (Hydroxylation process); 6,113,971 (Olive oil butter); 4,992,189 (Lubricants and lube additives); 5,576,027 (Hydroxylated milk); and 6,869,597 (Cosmetics).

Hydroxylated glycerolipids can be converted to estolides. Estolides consist of a glycerolipid in which a hydroxylated fatty acid constituent has been esterified to another fatty acid molecule. Conversion of hydroxylated glycerolipids to estolides can be carried out by warming a mixture of glycerolipids and fatty acids and contacting the mixture with a mineral acid, as described by Isbell et al., JAOCS 71(2):169-174 (1994). Estolides are useful in a variety of applications, including without limitation those reported in the following: U.S. Pat. Nos. 7,196,124 (Elastomeric materials and floor coverings); 5,458,795 (Thickened oils for high-temperature applications); 5,451,332 (Fluids for industrial applications); 5,427,704 (Fuel additives); and 5,380,894 (Lubricants, greases, plasticizers, and printing inks).

Another such chemical modification is olefin metathesis. In olefin metathesis a catalyst severs the alkylidene carbons in an alkene (olefin) and forms new alkenes by pairing each of them with different alkylidine carbons. The olefin metathesis reaction provides a mechanism for processes such as truncating unsaturated fatty acid alkyl chains at alkenes by ethenolysis, cross-linking fatty acids through alkene linkages by self-metathesis, and incorporating new functional groups on fatty acids by cross-metathesis with derivatized alkenes.

In conjunction with other reactions, such as transesterification and hydrogenation, olefin metathesis can transform unsaturated glycerolipids into diverse end products. These products include glycerolipid oligomers for waxes; short-chain glycerolipids for lubricants; homo- and hetero-bifunctional alkyl chains for chemicals and polymers; short-chain esters for biofuel; and short-chain hydrocarbons for jet fuel. Olefin metathesis can be performed on triacylglycerol and fatty acid derivatives, for example, using the catalysts and methods reported in U.S. Pat. No. 7,119,216, U.S. Patent Pub. No. 2010/0160506, and U.S. Patent Pub. No. 2010/0145086.

Olefin metathesis of bio-oils generally comprises adding a solution of Ru catalyst at a loading of about 10 to 250 ppm under inert conditions to unsaturated fatty acid esters in the presence (cross-metathesis) or absence (self-metathesis) of other alkenes. The reactions are typically allowed to proceed from hours to days and ultimately yield a distribution of alkene products. One example of how olefin metathesis may be performed on a fatty acid derivative is as follows: A solution of the first generation Grubbs Catalyst ([dichloro[2(1-methylethoxy-α-O)phenyl]methylene-α-C](tricyclohexyl-phosphine)ruthenium) in toluene at a catalyst loading of 222 ppm may be added to a vessel containing degassed and dried methyl oleate. Then the vessel may be pressurized with about 60 prig of ethylene gas and maintained at or below about 30° C. for 3 h, whereby approximately a 50% yield of methyl 9-decenoate may be produced.

Olefin metathesis of oils produced by the methods described herein can be performed in conjunction with one or more of the methods and/or materials, or to produce products, as reported in the following: Patent App. PCT/US07/081,427 (α-olefin fatty acids) and U.S. patent application Ser. Nos. 12/281,938 (petroleum creams), 12/281,931 (paintball gun capsules), 12/653,742 (plasticizers and lubricants), 12/422,096 (bifunctional organic compounds), and 11/795,052 (candle wax).

Other chemical reactions that can be performed on oleaginous yeast oils include reacting triacylglycerols with a cyclopropanating agent to enhance fluidity and/or oxidative stability, as reported in U.S. Pat. No. 6,051,539; manufacturing of waxes from triacylglycerols, as reported in U.S. Pat. No. 6,770,104; and epoxidation of triacylglycerols, as reported in “The effect of fatty acid composition on the acrylation kinetics of epoxidized triacylglycerols”, Journal of the American Oil Chemists' Society, 79:1, 59-63, (2001) and Free Radical Biology and Medicine, 37:1, 104-114 (2004).

The generation of oil-bearing oleaginous yeast biomass for fuel and chemical products as described above results in the production of delipidated biomass meal. Delipidated meal is a byproduct of preparing algal oil and is useful as animal feed for farm animals, e.g., ruminants, poultry, swine and aquaculture. The resulting meal, although of reduced oil content, still contains high quality proteins, carbohydrates, fiber, ash, residual oil and other nutrients appropriate for an animal feed. Because the cells are predominantly lysed by the oil separation process, the delipidated meal is easily digestible by such animals. Delipidated meal can optionally be combined with other ingredients, such as grain, in an animal feed. Because delipidated meal has a powdery consistency, it can be pressed into pellets using an extruder or expander or another type of machine, which are commercially available.

The invention, having been described in detail above, is exemplified in the following examples, which are offered to illustrate, but not to limit, the claimed invention.

### V. EXAMPLES

### Example 1

### Cultivation of Oleaginous Yeast

Oleaginous yeast strains used in this and subsequent Examples were obtained from either the Deutsche Sammlung von Mikroorganismen un Zellkulturen GmbH (DSMZ), located at Inhoffenstrabe 7B, 38124 Braunschweig, Germany, or Centraalbureau voor Schimmelscultures (CBS) Fungal Biodiversity Centre located at P.O. Box 85167, 3508 Utrecht, the Netherlands. One hundred eighty five oleaginous yeast strains were screened for growth rate and lipid production.

All strains were rendered axenic via streaking to single colonies on YPD agar (YPD medium as described below with 2% agar added) plates. Single colonies from the YPD plates of each strain was picked and grown to late log phase in YPD medium (10g bacto-yeast extract, 20g bacto-peptone and 20g glucose/1 L final volume in distilled water) on a rotary shaker at 200 rpm at 30° C.

For lipid productivity assessment, 2 mL of YPD medium was added to a 50 mL tared Bioreactor tube (MidSci, Inc.) and inoculated from a frozen stock of each strain. The tubes were then placed in a 30° C. incubator and grown for 24 hours, shaking at 200 rpm to generate a seed culture. After 24 hours, 8 mLs of Y1 medium (Yeast nitrogen base without amino acids, Difco) containing 0.1M phthalate buffer, pH 5.0 was added and mixed well by pipetting gently. The resulting culture was divided equally into a second, tared bioreactor tube. The resulting duplicate cultures of 5 mL each were then placed in a 30° C. incubator with 200 rpm agitation for 5 days. The cells were then harvested for lipid productivity and lipid profile. 3 mL of the culture was used for determination of dry cell weight and total lipid content (lipid productivity) and 1 mL was used for fatty acid profile determination. In either case, the cultures were placed into tubes and centrifuged at 3500 rpm for 10 minutes in order to pellet the cells. After decanting the supernatant, 2 mL of deionized water was added to each tube and used to wash the resulting cell pellet. The tubes were spun again at 3500 rpm for 10 minutes to pellet the washed cells, the supernatant was then decanted and the cell pellets were placed in a−70° C. freezer for 30 minutes. The tubes were then transferred into a lyophilizer overnight to dry. The following day, the weight of the conical tube plus the dried biomass resulting from the 3 mL culture was recorded and the resulting cell pellet was subjected to total lipid extraction using an Ankom Acid Hydrolysis system (according to the manufacturer's instructions) to determine total lipid content.

Of the 185 strains screened, 30 strains were chosen based on the growth rate and lipid productivity. The lipid productivity (expressed as percent lipid of dry cell weight) of these 30 strains are summarized below in Table 2.

TABLE 2
Lipid productivity of oleaginous yeast strains.

| | | % Lipid |
|---|---|---|
| Species | Collection No. | (DCW) |
| Rhodotorula terpenoidalis | CBS 8445 | 27 |
| Rhodotorula glutinus | DSMZ 70398 | 53.18 |
| Lipomyces tetrasporous | CBS 1810 | 51 |
| Lipomyces tetrasporous | CBS 7656 | 17.63 |
| Lipomyces tetrasporous | CBS 8724 | 18 |
| Cryptococcus curvatus | CBS 5324 | 53 |
| Cryptococcus curvatus | CBS 2755 | 48 |
| Rhodosporidium sphaerocarpum | CBS 2371 | 43 |
| Rhodotorula glutinus | CBS 4476 | 30.97 |
| Lipomyces tetrasporous | CBS 1808 | 29 |
| Trichosporon domesticum | CBS 8111 | 35.16 |
| Trichosporon sp. | CBS 7617 | 40.09 |
| Lipomyces tetrasporous | CBS 5911 | 27.63 |
| Lipomyces tetrasporous | CBS 5607 | 12.81 |
| Cryptococcus curvatus | CBS 570 | 38.64 |
| Cryptococcus curvatus | CBS 2176 | 40.57 |
| Cryptococcus curvatus | CBS 5163 | 35.26 |
| Torulaspora delbruekii | CBS 2924 | 40.00 |
| Rhodotorula toruloides | CBS 8761 | 36.52 |
| Geotrichum histeridarum | CBS 9892 | 33.77 |
| Yarrowia lipolytica | CBS 6012 | 29.21 |
| Geotrichum vulgare | CBS 10073 | 28.04 |
| Trichosporon montevideense | CBS 8261 | 25.60 |
| Lipomyces starkeyi | CBS 7786 | 25.43 |
| Trichosporon behrend | CBS 5581 | 23.93 |
| Trichosporon loubieri var. loubieri | CBS 8265 | 22.39 |
| Rhodosporidium toruloides | CBS 14 | 21.03 |
| Trichosporon brassicae | CBS 6382 | 20.34 |
| Rhodotorula aurantiaca | CBS 317 | 17.51 |
| Sporobolomyces alborubescens | CBS 482 | 10.09 |

Cell pellets resulting from 1 mL culture were subjected to direct transesterification and analysis by GC for fatty acid profile determination. A summary of the fatty acid profiles for 17 of the above yeast strains are summarized below in Table 3.

TABLE 3
Fatty acid profiles, in percent, of oleaginous yeast strains.

| | Collection | | | | | | | | | | | |
|---|---|---|---|---|---|---|---|---|---|---|---|---|
| Species | No. | C12:0 | C14:0 | C15:0 | C16:0 | C16:1 | C17:0 | C17:1 | C18:0 | C18:1 | C18:2 | >C20 |
| Rhodotorula | CBS 8445 | 0.06 | 0.8 | 0.02 | 27.44 | 0.67 | 0.03 | 0.03 | 5.6 | 59.44 | 3.37 | 2.13 |
| terpenoidalis | | | | | | | | | | | | |
| Rhodotorula | DSMZ | 0.05 | 1.55 | 0.09 | 27.34 | 0.34 | 0.23 | 0.08 | 10.47 | 44.68 | 11.65 | 2.23 |
| glutinus | 70398 | | | | | | | | | | | |
| Lipomyces | CBS 1810 | nd | 0.26 | 0.08 | 24.22 | 2.13 | 0.28 | 0.30 | 9.93 | 55.04 | 4.48 | 3.01 |
| tetrasporous | | | | | | | | | | | | |
| Lipomyces | CBS 76556 | nd | 0.293 | 0.212 | 28.14 | 4.24 | 0.37 | 0.66 | 6.61 | 48.48 | 8.33 | 1.178 |
| tetrasporous | | | | | | | | | | | | |
| Lipomyces | CBS 8724 | nd | 0.27 | 0.08 | 30.69 | 2.12 | 0.27 | 0.24 | 11.8 | 46.71 | 4.36 | 2.89 |
| tetrasporous | | | | | | | | | | | | |
| Cryptococcus | CBS 5324 | nd | 0.27 | 0.22 | 23.31 | 0.49 | 0.12 | 0.09 | 11.55 | 50.78 | 10.80 | 1.61 |
| curvatus | | | | | | | | | | | | |
| Cryptococcus | CBS 27556 | nd | 0.62 | 0.03 | 25.07 | 0.31 | 0.05 | 0.03 | 17.07 | 45.74 | 14.60 | 2.01 |
| curvatus | | | | | | | | | | | | |
| Rhodosporidium | CBS 2371 | 0.03 | 0.68 | 0.03 | 17.86 | 0.13 | 0.54 | 0.17 | 10.4 | 51.01 | 14.60 | 1.82 |
| sphaerocarpum | | | | | | | | | | | | |
| Rhodotorula | CBS 4476 | 0.021 | 0.47 | 0.02 | 24.64 | 0.16 | 0.064 | 0.27 | 13.73 | 42.46 | 16.29 | 1.642 |
| glutinus | | | | | | | | | | | | |
| Lipomyces | CBS 1808 | 0.01 | 0.40 | 0.12 | 26.64 | 3.11 | 0.25 | 0.39 | 7.39 | 54.15 | 3.96 | 2.34 |
| tetrasporous | | | | | | | | | | | | |
| Trichosporon | CBS 8111 | 0.066 | 0.486 | 0.10 | 23.19 | 0.11 | 0.37 | 0.033 | 30.65 | 29.75 | 11.66 | 3.414 |
| domesticum | | | | | | | | | | | | |
| Trichosporon | CBS 7617 | 0.046 | 0.527 | 0.063 | 24.26 | 0.187 | 0.171 | 0.026 | 19.61 | 41.95 | 9.97 | 2.61 |
| sp. | | | | | | | | | | | | |
| Lipomyces | CBS 5911 | 0.017 | 0.45 | 0.16 | 30.79 | 3.56 | 0.29 | 0.48 | 7.77 | 49.99 | 4.40 | 1.433 |
| tetrasporous | | | | | | | | | | | | |
| Lipomyces | CBS 5607 | nd | 0.35 | 0.17 | 37.56 | 3.0 | 0.328 | 0.40 | 9.31 | 42.36 | 4.28 | 1.376 |
| tetrasporous | | | | | | | | | | | | |
| Cryptococcus | CBS 570 | 0.017 | 0.21 | 0.09 | 12.78 | 0.13 | 0.147 | 0.09 | 19.6 | 53.17 | 8.42 | 4.01 |
| curvatus | | | | | | | | | | | | |
| Cryptococcus | CBS 2176 | 0.02 | 0.31 | 0.09 | 19.0 | 0.87 | 0.08 | 0.10 | 7.24 | 60.51 | 9.26 | 2.154 |
| curvatus | | | | | | | | | | | | |
| Cryptococcus | CBS 5163 | 0.019 | 0.34 | 0.06 | 22.7 | 0.70 | 0.13 | 0.10 | 10.65 | 51.36 | 10.34 | 2.24 |
| curvatus | | | | | | | | | | | | |

nd denotes none detected.

Lipid profile analysis was performed on additional strains of oleaginous yeast and several strains were found to produce a high percentage of C16:1 fatty acid including, Torulaspora delbruekii CBS 2924. This oleaginous yeast strain had approximately 40% lipid as a percentage of DCW and a fatty acid profile of: C12:0 (0.36%); C14:0 (1.36%); C15:0 (0.16%); C16:0 (10.82%); C16:1 (42.9%); C17:0 (0.11%); C18:0 (2.1%); C18:1 (35.81%); C18:2 (4.62%). This strain was found to have a particularly high percentage of C16:1 (palmitoleic acid) as part of its fatty acid profile. Four additional strains were identified as producing a high percentage 16:1: Yarrowia lipolytica CBS 6012 (10.10%); Yarrowia lipolytica CBS 6331 (14.80%), Yarrowia lipolytica CBS10144 (12.90%) and Yarrowia lipolytica CBS 5589 (14.20%). Palmitoleic acid is thought to have a health benefit by signaling to the body to produce less fat.

### Example 2

### Mechanical Extraction of Oil from Oleaginous Yeast Biomass

Yeast strain Rhodotorula glutinis (DSMZ-DSM 70398) was obtained from the Deutsche Sammlung von Mikroorganismen and Zellkulturen GmbH (German Collection of Microorganism and Cell Culture, Inhoffenstralβe 7B, 38124 Braunschweig, Germany. Cryopreserved cells were thawed and added to 50 mL YPD media (described above) with 1×DAS vitamin solution (1000×: 9 g/L tricine; 0.67 g/L thiamine-HCl; 0.01 g/L d-biotin; 0.008 cyannocobalamin; 0.02 calcium pantothenate; and 0.04 g/L p-Aminobenzoic acid) and grown at 30° C. with 200 rpm agitation for 18-24 hours until an OD reading was over 5 OD (A600). The culture was then transferred to 7-L fermentors and switched to YP1 medium (8.5 g/L Difco Yeast Nitrogen Base without Amino Acids and Ammonium Sulfate, 3 g/L Ammonium Sulfate, 4 g/L yeast extract) with 1×DAS vitamin solution. The cultures were sampled twice per day and assayed for OD (A600), dry cell weight (DCW) and lipid concentration. When the cultures reached over 50 g/L DCW, the cultures were harvested. Based on dry cell weight, the yeast biomass contained approximately 50% oil. Two samples of yeast biomass were subjected to direct transesterification and analyzed via GC/FID for a lipid profile. The results are expressed in Area Percent, and shown in Table 4, below.

TABLE 4
Lipid profile of transesterified yeast biomass samples.

| | C10:0 | C12:0 | C14:0 | C15:0 | C16:0 | C16:1 | C17:0 | C18:0 | C18:1 | C18:2 | C18:3α | ≧C:20 |
|---|---|---|---|---|---|---|---|---|---|---|---|---|
| Sample | 0.03 | 0.21 | 3.36 | 0.25 | 33.26 | 0.76 | 0.20 | 6.88 | 42.68 | 9.28 | 1.33 | 1.1 |
| 1 | | | | | | | | | | | | |
| Sample | 0.02 | 0.10 | 2.18 | 0.12 | 29.94 | 0.49 | 0.16 | 8.17 | 48.12 | 7.88 | 0.84 | 1.45 |
| 2 | | | | | | | | | | | | |

The harvested yeast broth was dried using three different methods for comparison: (1) tray dried in a forced air oven at 75° C. overnight; (2) dried on a drum dryer without concentration; and (3) the yeast broth was concentrated to 22% solids and the slurry was then dried on a drum dryer. Material from each of the three different drying conditions was heat conditioned and fed through a screw press for oil extraction. The press temperature was at 150° F. and the conditioned dried yeast biomass was held at about 190° F. until it was ready to be fed into the press.

The moisture content of the tray dried yeast was 1.45% and the dried yeast was then conditioned in an oven at 90° C. for 10 minutes. The moisture content after conditioning was 0.9%. The conditioned tray dried material was then fed into a bench-top Taby screw press (Taby Pressen Type 70 oil press with a 2.2 Hp motor and 70 mm screw diameter) for oil extraction. This material did not yield any significant amount of oil and heavy footing was observed with the press.

The moisture content of the drum dried yeast broth without concentration was 5.4% and the drum dried yeast was then conditioned in an oven at 90° C. for 20 minutes. The moisture content after conditioning was 1.4%. The conditioned drum dried yeast was then fed into a bench-top Taby screw press for oil extraction. This material oiled well, with minimal footing.

The moisture content of the drum dried concentrated yeast broth was 2.1% and the drum dried concentrated yeast was then conditioned in an oven at 90° C. for 20 minutes. The moisture content after conditioning was 1.0%. The conditioned drum dried concentrated yeast was then fed into a bench-top Taby screw press for oil extraction. This material oiled well, with minimal footing.

### Example 3

### Oleaginous Yeast Growth on Cellulosic Sugars and Carbon Utilization Screen

To assess if oleaginous yeast can grow on saccharified cellulosic sugars from cornstover, sorghum, Miscanthus or beet pulp, Rhodotorula glutinis (DSMZ-DSM 70398) were grown in YPD medium (described in Example 1) with 4% glucose at 30° C. with 200 rpm agitation overnight. This seed culture was then split into conditions containing 4% saccharified cellulosic sugars from cornstover, sorghum, Miscanthus, or beet pulp. Glucose condition was included as a positive control. The flasks were then grown at 30° C. with 200 rpm agitation for six days. The sugar concentration in the flasks was monitored daily and the flasks were fed to maintain a sugar level of 25 g/L. At the end of the sixth day, the cells were harvested and growth was determined. Rhodotorula glutinis was able to grow on all of the cellulosic sugars at a level that was either similar or better than the glucose control.

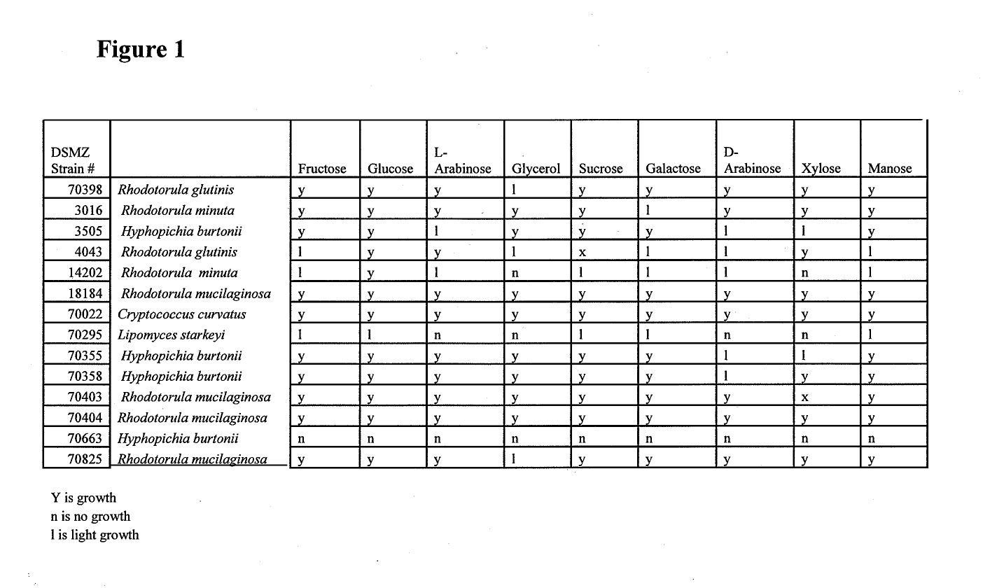

Additionally, carbon utilization screens were performed using agar plate assays using the method described in Example 1 above. The following oleaginous yeast strains were used in the screen: Rhodotorula glutinis (DSMZ-DSM 70398); Rhodotorula minuta (DSMZ-DSM 3016); Hyphopichia burtonii (DSMZ-DSM 3505); Rhodotorula glutinis (DSMZ-DSM 4043); Rhodotorula minuta (DSMZ-DSM 14202); Rhodotorula mucilaginosa (DSMZ-DSM 18184); Cryptococcus curvatus (DSMZ-DSM 70022); Lipomyces starkeyi (DSMZ-DSM 70295); Hyphopichia burtonii (DSMZ-DSM 70355); Hyphopichia burtonii (DSMZ-DSM 70358); Rhodotorula mucilaginosa (DSMZ-DSM 70403); Rhodotorula mucilaginosa(DSMZ-DSM 70404); Hyphopichia burtonii (DSMZ-DSM 70663); and Rhodotorula mucilaginosa (DSMZ-DSM 70825). The oleaginous yeast strains were screened for growth on agar plates containing 1% (w/v) of one of the following carbon sources: fructose, glucose, L-arabinose, glycerol, sucrose, galactose, D-arabinose, xylose and mannose. Each of the oleaginous yeast strains were streaked out onto a plate containing each of the carbon sources. The plates were allowed to grow at 30° C. for 7 days. At the end of seven days, the plates were checked visually for growth. The results of this assay are summarized in FIG. 1.

### Example 4

### Genotyping Oleaginous Yeast Strains

Genotyping of 48 different strains of oleaginous yeast was performed. Genomic DNA was isolated from each of the 48 different strains of oleaginous yeast biomass as follows. Cells (approximately 200 mg) were centrifuged from liquid cultures 5 minutes at 14,000×g. Cells were then resuspended in sterile distilled water, centrifuged 5 minutes at 14,000×g and the supernatant discarded. A single glass bead ˜2 mm in diameter was added to the biomass and tubes were placed at −80° C. for at least 15 minutes. Samples were removed and 150 μl of grinding buffer (1% Sarkosyl, 0.25 M Sucrose, 50 mM NaCl, 20 mM EDTA, 100 mM Tris-HCl, pH 8.0, RNase A 0.5 ug/ul) was added. Pellets were resuspended by vortexing briefly, followed by the addition of 40 ul of 5M NaCl. Samples were vortexed briefly, followed by the addition of 66 μl of 5% CTAB (Cetyl trimethylammonium bromide) and a final brief vortex. Samples were next incubated at 65° C. for 10 minutes after which they were centrifuged at 14,000×g for 10 minutes. The supernatant was transferred to a fresh tube and extracted once with 300 μl of Phenol:Chloroform:Isoamyl alcohol 12:12:1, followed by centrifugation for 5 minutes at 14,000×g. The resulting aqueous phase was transferred to a fresh tube containing 0.7 vol of isopropanol (˜190 μl), mixed by inversion and incubated at room temperature for 30 minutes or overnight at 4° C. DNA was recovered via centrifugation at 14,000×g for 10 minutes. The resulting pellet was then washed twice with 70% ethanol, followed by a final wash with 100% ethanol. Pellets were air dried for 20-30 minutes at room temperature followed by resuspension in 50 μl of 10 mM TrisCl, 1 mM EDTA (pH 8.0).

Five μl of total oleaginous yeast DNA, prepared as described above, was diluted 1:50 in 10 mM Tris, pH 8.0. PCR reactions, final volume 20 μl, were set up as follows. Ten μl of 2× iProof HF master mix (BIO-RAD) was added to 0.4 μl primer SZ5434 forward primer (5′ GTCCCTGCCCTTTGTACACAC-3′ (SEQ ID NO:1) at 10 mM stock concentration) and 0.4 μl primer SZ5435 reverse primer (5′-TTGATATGCTTAAGTTCAGCGGG-3′ (SEQ ID NO:2) at 10 mM stock concentration). The primers were selected based on sequence conservation between three prime regions of 18S and five prime regions of fungal 26S rRNA genes. By reference, the forward primer is identical to nucleotides 1632-1652 of Genbank Ascension # AY550243 and the reverse primer is identical to nucleotides 464271-464293 of Genbank Ascension # NC_{—}001144. Next, 5 μl of diluted total DNA and 3.2 μl dH₂O were added. PCR reactions were run as follows: 98° C., 45″; 98° C., 8″; 53° C., 12″; 72° C., 20″ for 35 cycles followed by 72° C. for 1 min and holding at 25° C. For purification of PCR products, 20 μl of 10 mM Tris, pH 8.0, was added to each reaction, followed by extraction with 40 μl of Phenol:Chloroform:isoamyl alcohol 12:12:1, vortexing and centrifuging at 14,000×g for 5 minutes. PCR reactions were applied to S-400 columns (GE Healthcare) and centrifuged for 2 minutes at 3,000×g. The resulting purified PCR products were cloned and transformed into E. coli using ZeroBlunt PCR4Blunt-TOPO vector kit (Invitrogen) according to manufacture's instructions. Sequencing reactions were carried out directly on ampicillin resistant colonies. Purified plasmid DNA was sequenced in both directions using M13 forward and reverse primers. Purified PCR products were subsequently TOPO cloned into PCR8/GW/TOPO and positive clones selected for on LB/Spec plates. Purified plasmid DNA was sequenced in both directions using M13 forward and reverse primers.

A list of the 48 strains of oleaginous yeast that were genotyped is summarized below in Table 5 along with their SEQ ID NOs.

TABLE 5
Genotyped oleaginous yeast strains.

| Strain Name | Strain Number | SEQ ID NO |
|---|---|---|
| Rhodotorula glutinis | DSMZ-DSM | SEQ ID NO: 3 |
| | 7098 | |
| Lipomyces tetrasporus | CBS 5911 | SEQ ID NO: 4 |
| Rhodotorula glutinis var. glutinis | CBS 3044 | SEQ ID NO: 5 |
| Lipomyces tetrasporus | CBS 8664 | SEQ ID NO: 6 |
| Lipomyces tetrasporus | CBS 1808 | SEQ ID NO: 7 |
| Lipomyces tetrasporus | CBS 1810 | SEQ ID NO: 7 |
| Lipomyces starkeyi | CBS 1809 | SEQ ID NO: 8 |
| Trichosporon montevideense | CBS 8261 | SEQ ID NO: 9 |
| Yarrowia lipolytica | CBS 6331 | SEQ ID NO: 10 |
| Cryptococcus curvatus | CBS 5324 | SEQ ID NO: 11 |
| Rhodotorula mucilaginosa var. | CBS 316 | SEQ ID NO: 12 |
| mucilaginosa | | |
| Cryptococcus curvatus | CBS 570 | SEQ ID NO: 11 |
| Cryptococcus curvatus | CBS 2176 | SEQ ID NO: 11 |
| Cryptococcus curvatus | CBS 2744 | SEQ ID NO: 11 |
| Cryptococcus curvatus | CBS 2754 | SEQ ID NO: 11 |
| Cryptococcus curvatus | CBS 2829 | SEQ ID NO: 11 |
| Cryptococcus curvatus | CBS 5163 | SEQ ID NO: 11 |
| Cryptococcus curvatus | CBS 5358 | SEQ ID NO: 11 |
| Trichosporon sp. | CBS 7617 | SEQ ID NO: 13 |
| Sporororbolomyces alborubescens | CBS 482 | SEQ ID NO: 14 |
| Rhodotorula glutinis var. glutinis | CBS 324 | SEQ ID NO: 15 |
| Rhodotorula glutinis var. glutinis | CBS 4476 | SEQ ID NO: 16 |
| Trichosporon behrend | CBS 5581 | SEQ ID NO: 17 |
| Geotrichum histeridarum | CBS 9892 | SEQ ID NO: 18 |
| Rhodotorula aurantiaca | CBS 8411 | SEQ ID NO: 19 |
| Cryptococcus curvatus | CBS 8126 | SEQ ID NO: 20 |
| Trichosporon domesticum | CBS 8111 | SEQ ID NO: 21 |
| Rhodotorula toruloides | CBS 8761 | SEQ ID NO: 22 |
| Rhodotorula terpendoidalis | CBS 8445 | SEQ ID NO: 23 |
| Yarrowia lipolytica | CBS 10144 | SEQ ID NO: 24 |
| Rhodotorula glutinis var. glutinis | CBS 5805 | SEQ ID NO: 25 |
| Yarrowia lipolytica | CBS 10143 | SEQ ID NO: 26 |
| Lipomyces tetrasporus | CBS 5607 | SEQ ID NO: 27 |
| Yarrowia lipolytica | CBS 5589 | SEQ ID NO: 28 |
| Lipomyces tetrasporus | CBS 8724 | SEQ ID NO: 29 |
| Rhodosporidium sphaerocarpum | CBS 2371 | SEQ ID NO: 30 |
| Trichosporon brassicae | CBS 6382 | SEQ ID NO: 31 |
| Cryptococcus curvatus | CBS 2755 | SEQ ID NO: 32 |
| Lipomyces tetrasporus | CBS 7656 | SEQ ID NO: 33 |
| Lipomyces starkeyi | CBS 7786 | SEQ ID NO: 34 |
| Yarrowia lipolytica | CBS 6012 | SEQ ID NO: 35 |
| Trichosporon loubieri var. loubieri | CBS 8265 | SEQ ID NO: 36 |
| Geotrichum vulgare | CBS 10073 | SEQ ID NO: 37 |
| Rhodosporidium toruloides | CBS 14 | SEQ ID NO: 38 |
| Rhodotorula glutinis var. glutinis | CBS 6020 | SEQ ID NO: 39 |
| Lipomyces orientalis | CBS 10300 | SEQ ID NO: 40 |
| Rhodotorula aurantiaca | CBS 317 | SEQ ID NO: 41 |
| Torulaspora delbrueckii | CBS 2924 | SEQ ID NO: 42 |

Although this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

All references cited herein, including patents, patent applications, and publications, are hereby incorporated by reference in their entireties, whether previously specifically incorporated or not. The publications mentioned herein are cited for the purpose of describing and disclosing reagents, methodologies and concepts that may be used in connection with the present invention. Nothing herein is to be construed as an admission that these references are prior art in relation to the inventions described herein. In particular, the following patent applications are hereby incorporated by reference in their entireties for all purposes: U.S. Provisional Application No. 60/941,581, filed Jun. 1, 2007, entitled “Production of Hydrocarbons in Microorganisms”; U.S. Provisional Application No. 60/959,174, filed Jul. 10, 2007, entitled “Production of Hydrocarbons in Microorganisms”; U.S. Provisional Application No. 60/968,291, filed Aug. 27, 2007, entitled “Production of Hydrocarbons in Microorganisms”; U.S. Provisional Application No. 61/024,069, filed Jan. 28, 2008, entitled “Production of Hydrocarbons in Microorganisms”; PCT Application No. PCT/US08/65563, filed Jun. 2, 2008, entitled “Production of Oil in Microorganisms”; U.S. patent application Ser. No. 12/131,783, filed Jun. 2, 2008, entitled “Use of Cellulosic Material for Cultivation of Microorganisms”; U.S. patent application Ser. No. 12/131,773, filed Jun. 2, 2008, entitled “Renewable Diesel and Jet Fuel from Microbial Sources”; U.S. patent application Ser. No. 12/131,793, filed Jun. 2, 2008, entitled “Sucrose Feedstock Utilization for Oil-Based Fuel Manufacturing”; U.S. patent application Ser. No. 12/131,766, filed Jun. 2, 2008, entitled “Glycerol Feedstock Utilization for Oil-Based Fuel Manufacturing”; U.S. patent application Ser. No. 12/131,804, filed Jun. 2, 2008, entitled “Lipid Pathway Modification in Oil-Bearing Microorganisms”; U.S. Patent Application No. 61/118,590, filed Nov. 28, 2008, entitled “Production of Oil in Microorganisms”; U.S. Provisional Patent Application No. 61/118,994, filed Dec. 1, 2008, entitled “Production of Oil in Microorganisms”; U.S. Provisional Patent Application No. 61/174,357, filed Apr. 3, 2009, entitled “Production of Oil in Microorganisms”; U.S. Provisional Patent Application No. 61/219,525, filed Jun. 23, 2009, entitled “Production of Oil in Microorganisms”; PCT Application No. PCT/US2009/066141, filed Nov. 30, 2009, entitled “Manufacturing of Tailored Oils in Recombinant Heterotrophic Microorganisms,” and PCT Application No. PCT/US2009/066142, filed Noverber 30, 2009, entitled “Production of Tailored Oils in Heterotrophic Microorganisms.”

What is claimed is:

1. A method for producing oil, said method comprising the steps of culturing an oleaginous yeast in a culture medium under conditions to produce oleaginous yeast comprising at least about 20% oil by dry weight; and extracting the oil from the oleaginous yeast.

2. The method of claim 1, wherein said oleaginous yeast comprises at least about 40% oil by dry weight.

3. The method of claim 1, wherein said oleaginous yeast is a strain of a genus selected from the group consisting of Candida, Cryptococcus, Debaromyces, Endomycopsis, Geotrichum, Hyphopichia, Lipomyces, Lypomyces, Pichia, Rodosporidium, Rhodotorula, Sporobolomyces, Starmerella, Torulaspora, Trichosporon, Wickerhamomyces, Yarrowia, and Zygoascus.

4. The method of claim 3, wherein said strain is selected from the group consisting of Candida apicola, Cryptococcus curvatus, Cryptococcus terricolus, Debaromyces hansenii, Endomycopsis vernalis, Geotrichum carabidarum, Geotrichum cucujoidarum, Geotrichum histeridarum, Geotrichum silvicola, Geotrichum vulgare, Hyphopichia burtonii, Lipomyces lipofer, Lypomyces orentalis, Lipomyces starkeyi, Lipomyces tetrasporous, Pichia mexicana, Rodosporidium sphaerocarpum, Rhodosporidium toruloides, Rhodotorula aurantiaca, Rhodotorula dairenensis, Rhodotorula diffluens, Rhodotorula glutinus, Rhodotorula glutinis var. glutinis, Rhodotorula gracilis, Rhodotorula graminis, Rhodotorula minuta, Rhodotorula mucilaginosa, Rhodotorula mucilaginosa var. mucilaginosa, Rhodotorula terpenoidalis, Rhodotorula toruloides, Sporobolomyces alborubescens, Starmerella bombicola, Torulaspora delbruekii, Torulaspora pretoriensis, Trichosporon behrend, Trichosporon brassicae, Trichosporon domesticum, Trichosporon laibachii, Trichosporon loubieri, Trichosporon loubieri var. loubieri, Trichosporon montevideense, Trichosporon pullulans, Trichosporon sp., Wickerhamomyces canadensis, Yarrowia lipolytica, Zygoascus meyerae, and any strain that has a 3′ region of fungal 18S or 5′ region of fungal 26S rRNA that is at least 80% identical to a corresponding region from any of the foregoing strains.

5. The method of claim 4, wherein the strain has a 3′ region of fungal 18S or 5′ region of fungal 26S rRNA that is at least 95% identical to a corresponding region from any of the foregoing strains.

6. The method of claim 1, wherein said culture medium comprises a carbon source selected from the group consisting of glucose, fructose, sucrose, lactose, galactose, xylose, mannose, rhamnose, arabinose, glycerol, and acetate.

7. The method of claim 1, wherein said culture medium comprises a carbon source selected from the group consisting of depolymerized sugar beet pulp, black liquor, corn starch, depolymerized cellulosic material, corn stover, sugar beet pulp, switchgrass, milk whey, molasses, potato, rice, sorghum, sugar cane, thick cane juice, sugar beet juice, and wheat.

8. The method of claim 1, wherein said oil is extracted by solvent extraction or by using an expeller press.

9. The method of claim 1, wherein said method comprises an additional step of converting said oil to a fuel.

10. The method of claim 9, wherein said fuel is selected from the group consisting of biodiesel, renewable diesel, and jet fuel.

11. The method of claim 10, wherein the fuel is renewable diesel and has a T10-T90 of at least 20° C. measured by ASTM D86 distillation.

12. The method of claim 1, comprising an additional step of subjecting said extracted oil to a chemical reaction to produce an oil-based material.

13. The method of claim 12, wherein said chemical reaction is selected from the group consisting of cracking, esterification, hydrogenation, hydroprocessing, hydrotreating, interesterification, isomerization, saponification, metathesis and transesterification.

14. The method of claim 13, wherein the chemical reaction is metathesis and the reaction is performed using the Grubbs catalyst.

15. The method of claim 1, wherein said oleaginous yeast produces oil having a lipid profile comprising an attribute selected from the group consisting of:
a) a lipid profile of below 10% C18:2;
b) a lipid profile of below 50% C18:1;
c) a lipid profile of at least 50% C18:1; and
d) a lipid profile of at least 10% C16.

16. An oil isolated from oleaginous yeast, said oil comprising at least 10% C16:1.
